SEQUENCE LISTING (58 sequences)

SEQ ID NO: 1 - protein, 53 aa, synthetic construct
DKCKKVYENYPVSKCQLANQCNYDCKLDKHARSGECFYDEKRNLQCICDYCEY

SEQ ID NO: 2 - DNA, 159 bp, synthetic construct
  source 1..159: mol_type = other DNA
gataagtgtaagaaggtatatgagaactatccagttagtaagtgtcaattggccaatcaatgcaactatgactgcaagttggacaagcacgccagaagtggtgagtgcttctacgatgagaagcggaatcttcaatgtatctgcgactactgtgaatac

SEQ ID NO: 3-4 - skipped (2 entries)

SEQ ID NO: 5 - protein, 4 aa, synthetic construct
KREA

SEQ ID NO: 6 - DNA, 12 bp, synthetic construct
  source 1..12: mol_type = other DNA
aagcgagaagcc

SEQ ID NO: 7 - protein, 12 aa, synthetic construct
KREAEAEAEAEA

SEQ ID NO: 8 - DNA, 36 bp, synthetic construct
  source 1..36: mol_type = other DNA
aagcgagaagccgaagcagaagcagaagcagaagcg

SEQ ID NO: 9 - protein, 108 aa, synthetic construct
DKCKKVYENYPVSKCQLANQCNYDCKLDKHARSGECFYDEKRNLQCICDYCEYKRDKCKKVYENYPVSKCQLANQCNYDCKLDKHARSGECFYDEKRNLQCICDYCEY

SEQ ID NO: 10 - DNA, 324 bp, synthetic construct
  source 1..324: mol_type = other DNA
gataagtgtaagaaggtatatgagaactatccagttagtaagtgtcaattggccaatcaatgcaactatgactgcaagttggacaagcacgccagaagtggtgagtgcttctacgatgagaagcggaatcttcaatgtatctgcgactactgtgaatacaagcgagataagtgtaagaaggtatatgagaactatccagttagtaagtgtcaattggccaatcaatgcaactatgactgcaagttggacaagcacgccagaagtggtgagtgcttctacgatgagaagcggaatcttcaatgtatctgcgactactgtgaatac

SEQ ID NO: 11 - protein, 438 aa, synthetic construct
DKCKKVYENYPVSKCQLANQCNYDCKLDKHARSGECFYDEKRNLQCICDYCEYKRDKCKKVYENYPVSKCQLANQCNYDCKLDKHARSGECFYDEKRNLQCICDYCEYKRDKCKKVYENYPVSKCQLANQCNYDCKLDKHARSGECFYDEKRNLQCICDYCEYKRDKCKKVYENYPVSKCQLANQCNYDCKLDKHARSGECFYDEKRNLQCICDYCEYKRDKCKKVYENYPVSKCQLANQCNYDCKLDKHARSGECFYDEKRNLQCICDYCEYKRDKCKKVYENYPVSKCQLANQCNYDCKLDKHARSGECFYDEKRNLQCICDYCEYKRDKCKKVYENYPVSKCQLANQCNYDCKLDKHARSGECFYDEKRNLQCICDYCEYKRDKCKKVYENYPVSKCQLANQCNYDCKLDKHARSGECFYDEKRNLQCICDYCEY

SEQ ID NO: 12 - DNA, 1314 bp, synthetic construct
  source 1..1314: mol_type = other DNA
gataagtgtaagaaggtatatgagaactatccagttagtaagtgtcaattggccaatcaatgcaactatgactgcaagttggacaagcacgccagaagtggtgagtgcttctacgatgagaagcggaatcttcaatgtatctgcgactactgtgaatacaagcgagataagtgtaagaaggtatatgagaactatccagttagtaagtgtcaattggccaatcaatgcaactatgactgcaagttggacaagcacgccagaagtggtgagtgcttctacgatgagaagcggaatcttcaatgtatctgcgactactgtgaatacaagcgagataagtgtaagaaggtatatgagaactatccagttagtaagtgtcaattggccaatcaatgcaactatgactgcaagttggacaagcacgccagaagtggtgagtgcttctacgatgagaagcggaatcttcaatgtatctgcgactactgtgaatacaagcgagataagtgtaagaaggtatatgagaactatccagttagtaagtgtcaattggccaatcaatgcaactatgactgcaagttggacaagcacgccagaagtggtgagtgcttctacgatgagaagcggaatcttcaatgtatctgcgactactgtgaatacaagcgagataagtgtaagaaggtatatgagaactatccagttagtaagtgtcaattggccaatcaatgcaactatgactgcaagttggacaagcacgccagaagtggtgagtgcttctacgatgagaagcggaatcttcaatgtatctgcgactactgtgaatacaagcgagataagtgtaagaaggtatatgagaactatccagttagtaagtgtcaattggccaatcaatgcaactatgactgcaagttggacaagcacgccagaagtggtgagtgcttctacgatgagaagcggaatcttcaatgtatctgcgactactgtgaatacaagcgagataagtgtaagaaggtatatgagaactatccagttagtaagtgtcaattggccaatcaatgcaactatgactgcaagttggacaagcacgccagaagtggtgagtgcttctacgatgagaagcggaatcttcaatgtatctgcgactactgtgaatacaagcgagataagtgtaagaaggtatatgagaactatccagttagtaagtgtcaattggccaatcaatgcaactatgactgcaagttggacaagcacgccagaagtggtgagtgcttctacgatgagaagcggaatcttcaatgtatctgcgactactgtgaatac

SEQ ID NO: 13 - protein, 110 aa, synthetic construct
DKCKKVYENYPVSKCQLANQCNYDCKLDKHARSGECFYDEKRNLQCICDYCEYKREADKCKKVYENYPVSKCQLANQCNYDCKLDKHARSGECFYDEKRNLQCICDYCEY

SEQ ID NO: 14 - DNA, 328 bp, synthetic construct
  source 1..328: mol_type = other DNA
taagtgtaagaaggtatatgagaactatccagttagtaagtgtcaattggccaatcaatgcaactatgactgcaagttggacaagcacgccagaagtggtgagtgcttctacgatgagaagcggaatcttcaatgtatctgcgactactgtgaatacaagcgagaagccgataagtgtaagaaggtatatgagaactatccagttagtaagtgtcaattggccaatcaatgcaactatgactgcaagttggacaagcacgccagaagtggtgagtgcttctacgatgagaagcggaatcttcaatgtatctgcgactactgtgaatac

SEQ ID NO: 15 - protein, 452 aa, synthetic construct
DKCKKVYENYPVSKCQLANQCNYDCKLDKHARSGECFYDEKRNLQCICDYCEYKREADKCKKVYENYPVSKCQLANQCNYDCKLDKHARSGECFYDEKRNLQCICDYCEYKREADKCKKVYENYPVSKCQLANQCNYDCKLDKHARSGECFYDEKRNLQCICDYCEYKREADKCKKVYENYPVSKCQLANQCNYDCKLDKHARSGECFYDEKRNLQCICDYCEYKREADKCKKVYENYPVSKCQLANQCNYDCKLDKHARSGECFYDEKRNLQCICDYCEYKREADKCKKVYENYPVSKCQLANQCNYDCKLDKHARSGECFYDEKRNLQCICDYCEYKREADKCKKVYENYPVSKCQLANQCNYDCKLDKHARSGECFYDEKRNLQCICDYCEYKREADKCKKVYENYPVSKCQLANQCNYDCKLDKHARSGECFYDEKRNLQCICDYCEY

SEQ ID NO: 16 - DNA, 1356 bp, synthetic construct
  source 1..1356: mol_type = other DNA
gataagtgtaagaaggtatatgagaactatccagttagtaagtgtcaattggccaatcaatgcaactatgactgcaagttggacaagcacgccagaagtggtgagtgcttctacgatgagaagcggaatcttcaatgtatctgcgactactgtgaatacaagcgagaagccgataagtgtaagaaggtatatgagaactatccagttagtaagtgtcaattggccaatcaatgcaactatgactgcaagttggacaagcacgccagaagtggtgagtgcttctacgatgagaagcggaatcttcaatgtatctgcgactactgtgaatacaagcgagaagccgataagtgtaagaaggtatatgagaactatccagttagtaagtgtcaattggccaatcaatgcaactatgactgcaagttggacaagcacgccagaagtggtgagtgcttctacgatgagaagcggaatcttcaatgtatctgcgactactgtgaatacaagcgagaagccgataagtgtaagaaggtatatgagaactatccagttagtaagtgtcaattggccaatcaatgcaactatgactgcaagttggacaagcacgccagaagtggtgagtgcttctacgatgagaagcggaatcttcaatgtatctgcgactactgtgaatacaagcgagaagccgataagtgtaagaaggtatatgagaactatccagttagtaagtgtcaattggccaatcaatgcaactatgactgcaagttggacaagcacgccagaagtggtgagtgcttctacgatgagaagcggaatcttcaatgtatctgcgactactgtgaatacaagcgagaagccgataagtgtaagaaggtatatgagaactatccagttagtaagtgtcaattggccaatcaatgcaactatgactgcaagttggacaagcacgccagaagtggtgagtgcttctacgatgagaagcggaatcttcaatgtatctgcgactactgtgaatacaagcgagaagccgataagtgtaagaaggtatatgagaactatccagttagtaagtgtcaattggccaatcaatgcaactatgactgcaagttggacaagcacgccagaagtggtgagtgcttctacgatgagaagcggaatcttcaatgtatctgcgactactgtgaatacaagcgagaagccgataagtgtaagaaggtatatgagaactatccagttagtaagtgtcaattggccaatcaatgcaactatgactgcaagttggacaagcacgccagaagtggtgagtgcttctacgatgagaagcggaatcttcaatgtatctgcgactactgtgaatac

SEQ ID NO: 17 - protein, 118 aa, synthetic construct
DKCKKVYENYPVSKCQLANQCNYDCKLDKHARSGECFYDEKRNLQCICDYCEYKREAEAEAEAEADKCKKVYENYPVSKCQLANQCNYDCKLDKHARSGECFYDEKRNLQCICDYCEY

SEQ ID NO: 18 - DNA, 354 bp, synthetic construct
  source 1..354: mol_type = other DNA
gataagtgtaagaaggtatatgagaactatccagttagtaagtgtcaattggccaatcaatgcaactatgactgcaagttggacaagcacgccagaagtggtgagtgcttctacgatgagaagcggaatcttcaatgtatctgcgactactgtgaatacaagcgagaagccgaagcagaagcagaagcagaagcggataagtgtaagaaggtatatgagaactatccagttagtaagtgtcaattggccaatcaatgcaactatgactgcaagttggacaagcacgccagaagtggtgagtgcttctacgatgagaagcggaatcttcaatgtatctgcgactactgtgaatac

SEQ ID NO: 19 - protein, 508 aa, synthetic construct
DKCKKVYENYPVSKCQLANQCNYDCKLDKHARSGECFYDEKRNLQCICDYCEYKREAEAEAEAEADKCKKVYENYPVSKCQLANQCNYDCKLDKHARSGECFYDEKRNLQCICDYCEYKREAEAEAEAEADKCKKVYENYPVSKCQLANQCNYDCKLDKHARSGECFYDEKRNLQCICDYCEYKREAEAEAEAEADKCKKVYENYPVSKCQLANQCNYDCKLDKHARSGECFYDEKRNLQCICDYCEYKREAEAEAEAEADKCKKVYENYPVSKCQLANQCNYDCKLDKHARSGECFYDEKRNLQCICDYCEYKREAEAEAEAEADKCKKVYENYPVSKCQLANQCNYDCKLDKHARSGECFYDEKRNLQCICDYCEYKREAEAEAEAEADKCKKVYENYPVSKCQLANQCNYDCKLDKHARSGECFYDEKRNLQCICDYCEYKREAEAEAEAEADKCKKVYENYPVSKCQLANQCNYDCKLDKHARSGECFYDEKRNLQCICDYCEY

SEQ ID NO: 20 - DNA, 1524 bp, synthetic construct
  source 1..1524: mol_type = other DNA
gataagtgtaagaaggtatatgagaactatccagttagtaagtgtcaattggccaatcaatgcaactatgactgcaagttggacaagcacgccagaagtggtgagtgcttctacgatgagaagcggaatcttcaatgtatctgcgactactgtgaatacaagcgagaagccgaagcagaagcagaagcagaagcggataagtgtaagaaggtatatgagaactatccagttagtaagtgtcaattggccaatcaatgcaactatgactgcaagttggacaagcacgccagaagtggtgagtgcttctacgatgagaagcggaatcttcaatgtatctgcgactactgtgaatacaagcgagaagccgaagcagaagcagaagcagaagcggataagtgtaagaaggtatatgagaactatccagttagtaagtgtcaattggccaatcaatgcaactatgactgcaagttggacaagcacgccagaagtggtgagtgcttctacgatgagaagcggaatcttcaatgtatctgcgactactgtgaatacaagcgagaagccgaagcagaagcagaagcagaagcggataagtgtaagaaggtatatgagaactatccagttagtaagtgtcaattggccaatcaatgcaactatgactgcaagttggacaagcacgccagaagtggtgagtgcttctacgatgagaagcggaatcttcaatgtatctgcgactactgtgaatacaagcgagaagccgaagcagaagcagaagcagaagcggataagtgtaagaaggtatatgagaactatccagttagtaagtgtcaattggccaatcaatgcaactatgactgcaagttggacaagcacgccagaagtggtgagtgcttctacgatgagaagcggaatcttcaatgtatctgcgactactgtgaatacaagcgagaagccgaagcagaagcagaagcagaagcggataagtgtaagaaggtatatgagaactatccagttagtaagtgtcaattggccaatcaatgcaactatgactgcaagttggacaagcacgccagaagtggtgagtgcttctacgatgagaagcggaatcttcaatgtatctgcgactactgtgaatacaagcgagaagccgaagcagaagcagaagcagaagcggataagtgtaagaaggtatatgagaactatccagttagtaagtgtcaattggccaatcaatgcaactatgactgcaagttggacaagcacgccagaagtggtgagtgcttctacgatgagaagcggaatcttcaatgtatctgcgactactgtgaatacaagcgagaagccgaagcagaagcagaagcagaagcggataagtgtaagaaggtatatgagaactatccagttagtaagtgtcaattggccaatcaatgcaactatgactgcaagttggacaagcacgccagaagtggtgagtgcttctacgatgagaagcggaatcttcaatgtatctgcgactactgtgaatac

SEQ ID NO: 21 - protein, 517 aa, synthetic construct
MQFNWDIKTVASILSALTLAQASDQEAIAPEDSHVVKLTEATFESFITSNPHVLAEFFAPWCGHCKKLGPELVSAAEILKDNEQVKIAQIDCTEEKELCQGYEIKGYPTLKVFHGEVEVPSDYQGQRQSQSIVSYMLKQSLPPVSEINATKDLDDTIAEAKEPVIVQVLPEDASNLESNTTFYGVAGTLREKFTFVSTKSTDYAKKYTSDSTPAYLLVRPGEEPSVYSGEELDETHLVHWIDIESKPLFGDIDGSTFKSYAEANIPLAYYFYENEEQRAAAADIIKPFAKEQRGKINFVGLDAVKFGKHAKNLNMDEEKLPLFVIHDLVSNKKFGVPQDQELTNKDVTELIEKFIAGEAEPIVKSEPIPEIQEEKVFKLVGKAHDEVVFDESKDVLVKYYAPWCGHCKRMAPAYEELATLYANDEDASSKVVIAKLDHTLNDVDNVDIQGYPTLILYPAGDKSNPQLYDGSRDLESLAEFVKERGTHKVDALALRPVEEEKEAEEEAESEADAHDEL

SEQ ID NO: 22 - DNA, 1554 bp, synthetic construct
  source 1..1554: mol_type = other DNA
atgcaattcaactgggatattaaaactgtggcaagtattttgtccgctctcacactagcacaagcaagtgatcaggaggctattgctccagaggactctcatgtcgtcaaattgactgaagccacttttgagtctttcatcaccagtaatcctcacgttttggcagagttttttgccccttggtgtggtcactgtaagaagttgggccctgaacttgtttctgctgccgagattttaaaggacaatgagcaggttaagattgctcaaattgattgtacggaggagaaggaattatgtcaaggctacgaaattaaagggtatcctactttgaaggtgttccatggtgaggttgaggtcccaagtgactatcaaggtcaaagacagagccaaagcattgtcagctatatgctaaagcagagtttaccccctgtcagtgaaatcaatgcaaccaaagatttagacgacacaatcgccgaggcaaaagagcccgtgattgtgcaagtactaccggaagatgcatccaacttggaatctaacaccacattttacggagttgccggtactctcagagagaaattcacttttgtctccactaagtctactgattatgccaaaaaatacactagcgactcgactcctgcctatttgcttgtcagacctggcgaggaacctagtgtttactctggtgaggagttagatgagactcatttggtgcactggattgatattgagtccaaacctctatttggagacattgacggatctaccttcaaatcatacgctgaagctaacatccctttagcctactatttctatgagaacgaagaacaacgtgctgctgctgccgatattattaaaccttttgctaaagagcaacgtggcaaaattaactttgttggcttagatgccgttaaattcggtaagcatgccaagaacttaaacatggatgaagagaaactccctctatttgtcattcatgatttggtgagcaacaagaagtttggagttcctcaagaccaagaattgacgaacaaagatgtgaccgagctgattgagaaattcatcgcaggagaggcagaaccaattgtgaaatcagagccaattccagaaattcaagaagagaaagtcttcaagctagtcggaaaggcccacgatgaagttgtcttcgatgaatctaaagatgttctagtcaagtactacgccccttggtgtggtcactgtaagagaatggctcctgcttatgaggaattggctactctttacgccaatgatgaggatgcctcttcaaaggttgtgattgcaaaacttgatcacactttgaacgatgttgacaacgttgatattcaaggttatcctactttgatcctttatccagctggtgataaatccaatcctcaactgtatgatggatctcgtgacctagaatcattggctgagtttgtaaaggagagaggaacccacaaagtggatgccctagcactcagaccagtcgaggaagaaaaggaagctgaagaagaagctgaaagtgaggcagacgctcacgacgagctttaa

SEQ ID NO: 23 - protein, 579 aa, synthetic construct
MASSSTSISLLLFVSFILLLVNSRAENASSGSDLDEELAFLAAEESKEQSHGGGSYHEEEHDHQHRDFENYDDLEQGGGEFHHGDHGYEEEPLPPVDEKDVAVLTKDNFTEFVGNNSFAMVEFYAPWCGACQALTPEYAAAATELKGLAALAKIDATEEGDLAQKYEIQGFPTVFLFVDGEMRKTYEGERTKDGIVTWLKKKASPSIHNITTKEEAERVLSAEPKLVFGFLNSLVGSESEELAAASRLEDDLSFYQTASPDIAKLFEIETQVKRPALVLLKKEEEKLARFDGNFTKTAIAEFVSANKVPLVINFTREGASLIFESSVKNQLILFAKANESEKHLPTLREVAKSFKGKFVFVYVQMDNEDYGEAVSGFFGVTGAAPKVLVYTGNEDMRKFILDGELTVNNIKTLAEDFLADKLKPFYKSDPLPENNDGDVKVIVGNNFDEIVLDESKDVLLEIYAPWCGHCQSFEPIYNKLGKYLKGIDSLVVAKMDGTSNEHPRAKADGFPTILFFPGGNKSFDPIAVDVDRTVVELYKFLKKHASIPFKLEKPATPEPVISTMKSDEKIEGDSSKDEL

SEQ ID NO: 24 - DNA, 1740 bp, synthetic construct
  source 1..1740: mol_type = other DNA
atggcttcttcttctacttctatttctttgttgttgttcgtttctttcatcttgttgttggttaattctagagctgaaaacgcttcttctggttctgatttggatgaagaattggcttttcttgctgctgaagaatctaaagaacaatctcatggtggtggttcttatcatgaagaagaacatgatcatcaacatagagattttgaaaactacgatgatttggaacaaggtggtggtgaatttcatcatggtgaccatggttacgaagaagaaccattgccaccagttgatgaaaaagatgttgctgttttgactaaagataacttcactgaatttgtcggtaataactctttcgctatggttgaattttacgctccatggtgtggtgcttgtcaagctttgactcctgaatatgctgctgctgctactgaattgaaaggtttggctgctttggctaagattgatgctactgaagaaggtgacttggctcaaaagtatgaaattcaaggttttcctactgttttcttgtttgttgatggtgaaatgagaaagacttatgaaggtgaaagaactaaggatggtattgttacttggttgaaaaagaaagcttctccttctattcataacattactactaaggaagaggctgaaagagttttgtctgctgaaccaaagttggtttttggttttcttaactctttggttggttctgaatctgaagaattggccgctgcttctagattggaagatgatttgtctttttaccaaactgcttctcctgatattgctaaattgttcgaaattgaaacccaagttaagcgtcctgctttggttttgttgaaaaaggaagaagaaaagttggctagatttgatggtaattttactaagactgctatcgctgaatttgtttctgctaataaggttccattggttattaatttcaccagagaaggtgcttctttgattttcgaatcttctgttaagaaccaattgattttgttcgctaaagctaatgaatctgaaaagcatttgcctactttgagagaagttgctaagtctttcaaaggtaaattcgttttcgtttacgttcaaatggataatgaagattacggtgaagctgtttctggtttctttggtgttactggtgctgctccaaaggttttggtttatactggtaacgaagatatgagaaagttcattttggatggtgaattgactgttaacaatattaagactctggctgaagattttcttgctgataagttgaaaccattctacaagtctgatccattgcctgaaaacaacgatggtgacgttaaggttattgttggtaacaacttcgatgaaattgttttggatgaatctaaggatgttttgttggaaatctatgctccatggtgcggtcattgtcaatcttttgaaccaatctataacaagttgggtaaatacttgaagggtattgattctttggttgttgctaaaatggatggtacttctaacgaacatccaagagctaaagctgatggttttcctaccattttgtttttccctggtggtaataagtctttcgatcctattgctgttgatgttgatagaactgttgttgaattgtataagttcttgaagaagcatgcttctattcctttcaagttggaaaagccagctactccagaacctgttatttctactatgaagtctgatgaaaagatcgaaggtgactcttctaaggatgaattgtaa

SEQ ID NO: 25 - protein, 331 aa, synthetic construct
MPVDSSHKTASPLPPRKRAKTEEEKEQRRVERILRNRRAAHASREKKRRHVEFLENHVVDLESALQESAKATNKLKEIQDIIVSRLEALGGTVSDLDLTVPEVDFPKSSDLEPMSDLSTSSKSEKASTSTRRSLTEDLDEDDVAEYDDEEEDEELPRKMKVLNDKNKSTSIKQEKLNELPSPLSSDFSDVDEEKSTLTHLKLQQQQQQPVDNYVSTPLSLPEDSVDFINPGNLKIESDENFLLSSNTLQIKHENDTDYITTAPSGSINDFFNSYDISESNRLHHPAVMTDSSLHITAGSIGFFSLIGGGESSVAGRRSSVGTYQLTCIAIR

SEQ ID NO: 26 - DNA, 993 bp, synthetic construct
  source 1..993: mol_type = other DNA
atgcccgtagattcttctcataagacagctagcccacttccacctcgtaaaagagcaaagacggaagaagaaaaggagcagcgtcgagtggaacgtatcctacgtaataggagagcggcccatgcttccagagagaagaaacgaagacacgttgaatttctggaaaaccacgtcgtcgacctggaatctgcacttcaagaatcagccaaagccactaacaagttgaaagaaatacaagatatcattgtttcaaggttggaagccttaggtggtaccgtctcagatttggatttaacagttccggaagtcgattttcccaaatcttctgatttggaacccatgtctgatctctcaacttcttcgaaatcggagaaagcatctacatccactcgcagatctttgactgaggatctggacgaagatgacgtcgctgaatatgacgacgaagaagaggacgaagagttacccaggaaaatgaaagtcttaaacgacaaaaacaagagcacatctatcaagcaggagaagttgaatgaacttccatctcctttgtcatccgatttttcagacgtagatgaagaaaagtcaactctcacacatttaaagttgcaacagcaacaacaacaaccagtagacaattatgtttctactcctttgagtcttccggaggattcagttgattttattaacccaggtaacttaaaaatagagtccgatgagaacttcttgttgagttcaaatactttacaaataaaacacgaaaatgacaccgactacattactacagctccatcaggttccatcaatgatttttttaattcttatgacattagcgagtcgaatcggttgcatcatccagcagtgatgacggattcatctttacacattacagcaggctccatcggctttttctctttgattggggggggggaaagttctgtagcagggaggcgcagttcagttggcacatatcagttgacatgcatagcgatcagg

SEQ ID NO: 27 - protein, 469 aa, synthetic construct
MGKGAIKEEESEKKRKTWRWPLATLVVVFLAVAVSSRTNSNVGFFFSDRNSCSCSLQKTGKYKGMIEDCCCDYETVDNLNTEVLNPLLQDLVTTPFFRYYKVKLWCDCPFWPDDGMCRLRDCSVCECPENEFPEPFKKPFVPGLPSDDLKCQEGKPQGAVDRTIDNRAFRGWVETKNPWTHDDDTDSGEMSYVNLQLNPERYTGYTGPSARRIWDSIYSENCPKYSSGETCPEKKVLYKLISGLHSSISMHIAADYLLDESRNQWGQNIELMYDRILRHPDRVRNMYFTYLFVLRAVTKATAYLEQAEYDTGNHAEDLKTQSLIKQLLYSPKLQTACPVPFDEAKLWQGQSGPELKQQIQKQFRNISALMDCVGCEKCRLWGKLQVQGLGTALKILFSVGNQDIGDQTLQLQRNEVIALVNLLNRLSESVKMVHDMSPDVERLMEDQIAKVSAKPARLRRIWDLAVSFW

SEQ ID NO: 28 - DNA, 1410 bp, synthetic construct
  source 1..1410: mol_type = other DNA
atgggtaaaggtgctattaaggaagaagaatctgaaaagaagagaaaaacttggagatggcctttggctactttggttgttgttttcttggctgttgctgtttcttctagaactaactctaacgttggtttctttttctctgatagaaattcttgttcctgttctttgcaaaaaactggtaaatacaagggtatgattgaagattgttgttgtgattatgagactgttgataacttgaatactgaagttttgaaccctttgttgcaagatttggttactactccatttttcagatactacaaagttaagttgtggtgtgattgtccattctggccagatgatggtatgtgtagattgagagattgttctgtttgtgaatgtccagaaaacgaatttcctgaaccattcaaaaagcctttcgttcctggtttgccatctgatgatttgaaatgtcaagaaggtaaaccacaaggtgctgttgatagaactattgataacagagcttttagaggttgggttgaaactaaaaacccttggactcatgatgatgatactgattctggtgaaatgtcttatgttaatttgcaattgaacccagaaagatacactggttacactggtccttctgctagaagaatttgggattctatctattctgaaaactgtccaaagtactcttctggtgaaacttgtccagaaaagaaagttttgtataagttgatctccggtttgcattcttctatttctatgcatattgctgctgattatttgttggatgaatctagaaatcagtggggtcaaaacattgaattgatgtatgatagaatcctgagacatccagatagagttagaaatatgtatttcacttacctgttcgttttgagagctgttactaaagctactgcttatttggaacaagctgaatacgatactggtaaccatgctgaagatttgaaaactcaatctttgattaagcagttgttgtattctcctaaattgcaaactgcttgtccagttccttttgatgaagctaagttgtggcaaggtcaatctggtccagaattgaaacaacaaattcaaaaacagttcagaaacatctctgctttgatggattgtgttggttgtgaaaagtgtagattgtggggtaaattgcaagttcaaggtttgggtactgctttgaaaattttgttttctgttggtaaccaggatatcggtgaccaaactttgcaattgcaaagaaacgaagttattgctttggttaatttgttgaacagattgtctgaatctgttaagatggttcatgatatgtctccagatgttgaaagattgatggaagatcaaattgctaaagtttctgctaaacctgctagattgagaagaatttgggacttggctgtttctttctggtaa

SEQ ID NO: 29 - protein, 472 aa, synthetic construct
MAETDVGSVKGKEKGSGKRWILLIGAIAAVLLAVVVAVFLNTQNSSISEFTGKICNCRQAEQQKYIGIVEDCCCDYETVNRLNTEVLNPLLQDLVKTPFYRYFKVKLWCDCPFWPDDGMCRLRDCSVCECPESEFPEVFKKPLSQYNPVCQEGKPQATVDRTLDTRAFRGWTVTDNPWTSDDETDNDEMTYVNLRLNPERYTGYIGPSARRIWEAIYSENCPKHTSEGSCQEEKILYKLVSGLHSSISVHIASDYLLDEATNLWGQNLTLLYDRVLRYPDRVQNLYFTFLFVLRAVTKAEDYLGEAEYETGNVIEDLKTKSLVKQVVSDPKTKAACPVPFDEAKLWKGQRGPELKQQLEKQFRNISAIMDCVGCEKCRLWGKLQILGLGTALKILFTVNGEDNLRHNLELQRNEVIALMNLLHRLSESVKYVHDMSPAAERIAGGHASSGNSFWQRIVTSIAQSKAVSGKRS

SEQ ID NO: 30 - DNA, 1419 bp, synthetic construct
  source 1..1419: mol_type = other DNA
atggctgaaactgatgttggttctgttaagggtaaagaaaagggttctggtaaaagatggattttgttgattggtgctattgctgctgttttgttggctgttgttgttgctgttttcttgaacactcaaaactcttctatttctgagtttactggtaaaatctgtaactgtagacaagctgaacaacaaaagtacattggtattgttgaagattgttgttgtgattatgagactgttaacagattgaacactgaagttttgaacccattgttgcaagatttggttaagactccattctacagatactttaaggttaagttgtggtgtgattgtcctttctggccagatgatggtatgtgtagattgagagattgttctgtttgtgaatgtccagaatctgaatttcctgaagttttcaagaaacctttgtctcaatataacccagtttgtcaagaaggtaaaccacaagctactgttgatagaactttggatactagagctttcagaggttggactgttactgataatccttggacttctgatgatgaaactgataacgatgaaatgacttatgttaacttgagattgaacccagaaagatacactggttatattggtccatctgctagaagaatttgggaagctatctattctgaaaattgtccaaaacatacctctgaaggttcttgtcaagaagaaaagattttgtataagctggtttctggtttgcattcttctatttccgttcatattgcttctgattacttgttggatgaagctactaacttgtggggtcaaaacttgactttgttgtatgatagagttttgagatacccagatagagttcaaaacttgtactttactttcttgttcgttttgagagctgttactaaagctgaagattacttgggtgaagctgaatacgaaactggtaacgttattgaagatttgaaaactaaatctctggtcaagcaagttgtttctgatccaaaaactaaggctgcttgtccagttccatttgatgaagctaagttgtggaagggtcaaagaggtccagaattgaagcaacaattggaaaagcaatttcgtaacatttctgctattatggattgtgttggttgtgaaaaatgtagattgtggggtaaattgcaaattttgggtttgggtactgctttgaaaattttgtttactgttaacggtgaggataatttgagacataacttggaattgcaaagaaacgaagttattgctttgatgaatttgttgcatagattgtctgaatctgttaaatacgttcatgatatgtctcctgctgctgaaagaattgctggtggtcatgcttcttctggtaattctttttggcaaagaattgttacttccattgctcaatctaaagctgtttctggtaaaagatcctaa

SEQ ID NO: 31 - protein, 191 aa, synthetic construct
MGEKPWQPLLQSFEKLSNCVQTHLSNFIGIKNTPPSSQSTIQNPIISLDSSPPIATNSSSLQKLPLKDKSTGPVTKEDLGRATWTFLHTLAAQYPEKPTRQQKKDVKELMTILSRMYPCRECADHFKEILRSNPAQAGSQEEFSQWLCHVHNTVNRSLGKLVFPCERVDARWGKLECEQKSCDLHGTSMDF

SEQ ID NO: 32 - DNA, 576 bp, synthetic construct
  source 1..576: mol_type = other DNA
atgggtgaaaaaccatggcaaccattgttgcaatctttcgaaaagttgtctaattgtgttcaaactcatttgtctaacttcattggtattaagaacactccaccatcttctcaatctactattcaaaaccctattatctctttggattcttctccaccaattgctactaattcttcttctttgcaaaagttgcctttgaaggataagtctactggtccagttactaaggaagatttgggtagagctacttggacttttcttcatactttggctgctcaataccctgaaaaacctactagacaacaaaagaaagatgttaaggaattgatgactatcttgtctagaatgtatccatgtagagaatgtgctgatcatttcaaagaaattttgagatccaaccctgctcaagctggttctcaagaagaattttctcaatggttgtgtcatgttcataacactgttaatagatccttgggtaaattggttttcccttgtgaaagagttgatgctagatggggtaaattggaatgtgaacaaaaatcttgtgacttgcatggtacttctatggatttttaa

SEQ ID NO: 33 - protein, 205 aa, synthetic construct
MIKFNKRVATLTATLLSFIVLYTLFNSGARFANQLDQPVPLKTPELIIPNQSTKNDAPLPFMPKMANETLKAELGNASWKLFHTILARYPESPSENQKSTLNDYIYLFAQVYPCGDCARHFNLLLQKYPPQLSSRQVAAVWGCHIHNQVNKRLEKPQYDCSNILEDYDCGCGSDEKEVDDTLNNETMEHLQSIKITEKENEQFGR

SEQ ID NO: 34 - DNA, 615 bp, synthetic construct
  source 1..615: mol_type = other DNA
atgataacattcaacaaacgaatagcaacattagcggcaacgttattttcattcattgtgctttatactctctttaacagtggtgctcaattttccaaccaactagatcagcctgttcccctcaaaactccagaactcatcataccgaatcagagtactgagaatgatccccctcttccattcatgccaaaaatggctaacgaaactttgaaagcagaacttggaaatgcttcctggaaactctttcacactattcttgctagatatcctgaatccccatcggagaatcaaaaatcaaccttaaatgactacatttatttgtttgcacaggtttatccatgtggagactgtgcaagacatttcaatttattgctgcagaaataccctccacaattgtcctcaagacaggtggctgcagtgtggggatgtcatattcacaatcaggtcaataagagattggagaaaccacaatacgactgctccaatattctagaggattacgattgtggatgtggctctgatgaaaaggaagtagatgacactctgaataacgaaacaatagaacacttgcaaagtatcaaaattactgaaaaagagagtgaacaatttggtcga

SEQ ID NO: 35 - protein, 527 aa, synthetic construct
MRIVRSLAVTITCYCITALANPQIPFDGNYTEITVPDTEVNIGQIVDINHEIKPKLVELVNTDFFKYYKLNLWKPCPFWNGDEGFCKYKDCSVDFITDWSQVPDIWQPDQLGKLGDNTVHKDKGQDENELSSNDYCALDKDDDEDLVYVNLIDNPERFTGYGGQQSESIWTAVYDENCFQPNEGSQLGQVEDLCLEKQIFYRLVSGLHSSISTHLTNEYLNLKNGEYEPNLKQFMIKVGYFTERIQNLHLNYVLVLKSLIKLQEYNVIENLPLDDSLKAGLSGLISQGAQNINQTDDYLFNEKVLFQNDQNDDLKNEFRDKFRNVTRLMDCVHCERCKLWGKLQTTGYGTALKILFDLKNPNDSINLKRVELVALVNTFHRLSKSVESIENFEKLYKIQPPTQDHPSPSSESLDVFDNEDEQNFFDSFSVDQTVTSSKEPPEEIKSKPVGKAEYKKTNSCPSSGSKSIKEAFHEELYAFIDAIGFILNSYRTLPKLLYTLFLVKSSELWDIFIGTQRHRDSTYRVDL

SEQ ID NO: 36 - DNA, 1598 bp, synthetic construct
  source 1..1598: mol_type = other DNA
atgaggatagtaaggagcgtagctatcgcaatagcctgtcattgtataacagcgttagcaaaccctcaaatcccttttgacggcaactacaccgagatcatcgtgccagataccgaagttaacatcggacagattgtagatattaaccacgaaataaaacccaaactggtggaactggtcaacacagacttcttcaaatattacaaattaaacctatggaaaccatgtccgttttggaatggtgatgagggattctgcaagtataaggattgctctgttgactttatcactgattggtcccaggtgcctgatatctggcaaccagaccaattgggtaagcttggagataacacggtacataaggataagggccaagatgaaaatgagctgtcctcaaatgattattgcgctttggataaagacgacgatgaagatttagtatatgtcaatttgattgataaccctgaaagattcaccggttatggtggtcagcaatctgaatctatttggactgcggtctatgatgagaactgtttccagccgaatgaaggatcacaattgggtcaagttgaagacctctgtttggagaaacaaatcttttaccgattggtttctggtttgcattctagtatctccacccacctcacaaacgaatatctgaatttgaaaaatggagcatacgaaccaaatttgaaacagttcatgatcaaagttgggtattttactgaaagaatccaaaacttacatctcaattatgtccttgtattgaagtcactaataaagctacaagaatacaatgttatcgacaatctacctctcgatgactctttgaaagctggtcttagcggtttaatatctcaaggagcacagggtattaaccagagttctgatgattatctatttaacgagaaggttcttttccaaaatgaccaaaatgatgatttgaaaaatgaatttcgtgacaaattccgcaacgtgactagattaatggattgtgtccattgcgagagatgcaaattatggggaaaattgcaaactacagggtacgggactgcattgaagattctatttgatttgaagaatcctaatgactccatcaatttaaagagagttgagttagttgctctagtcaacacattccatagattgtccaaatctgttgaaagcattgaaaactttgaaaaactatataagattcaaccgccaacgcaggatcgtgcatcagcgtcgtccgaatccttaggccttttcgataacgaagatgaacaaaatctcctcaactcgttttcggttgatcaggcagtcatttcatcgaaagaggcaccagaagaaatcaaaagcaaacctgttggaaaagccgcatataaacaaaacagttgtccatcattgggttcaaaatctatcaaagaagcattccatgaagaacttcacgcatttattgatgcaattggatttatattgaactcttacaggactttgcccaagctgttgtacacacttttcctcgttaaatcatctgaattatgggacattttcattggcactcaaaggcaccgagataccacatatagagtagacttgtaagcggccgccagctt

SEQ ID NO: 37 - protein, 678 aa, synthetic construct
MLSLKPSWLTLAALMYAMLLVVVPFAKPVRADDVESYGTVIGIDLGTTYSCVGVMKSGRVEILANDQGNRITPSYVSFTEDERLVGDAAKNLAASNPKNTIFDIKRLIGMKYDAPEVQRDLKRLPYTVKSKNGQPVVSVEYKGEEKSFTPEEISAMVLGKMKLIAEDYLGKKVTHAVVTVPAYFNDAQRQATKDAGLIAGLTVLRIVNEPTAAALAYGLDKTGEERQIIVYDLGGGTFDVSLLSIEGGAFEVLATAGDTHLGGEDFDYRVVRHFVKIFKKKHNIDISNNDKALGKLKREVEKAKRTLSSQMTTRIEIDSFVDGIDFSEQLSRAKFEEINIELFKKTLKPVEQVLKDAGVKKSEIDDIVLVGGSTRIPKVQQLLEDYFDGKKASKGINPDEAVAYGAAVQAGVLSGEEGVDDIVLLDVNPLTLGIETTGGVMTTLINRNTAIPTKKSQIFSTAADNQPTVLIQVYEGERALAKDNNLLGKFELTGIPPAPRGTPQVEVTFVLDANGILKVSATDKGTGKSESITINNDRGRLSKEEVDRMVEEAEKYAAEDAALREKIEARNALENYAHSLRNQVTDDSETGLGSKLDEDDKETLTDAIKDTLEFLEDNFDTATKEELDEQREKLSKIAYPITSKLYGAPEGGTPPGGQGFDDDDGDFDYDYDYDHDEL

SEQ ID NO: 38 - DNA, 2037 bp, synthetic construct
  source 1..2037: mol_type = other DNA
atgctgtcgttaaaaccatcttggctgactttggcggcattaatgtatgccatgctattggtcgtagtgccatttgctaaacctgttagagctgacgatgtcgaatcttatggaacagtgattggtatcgatttgggtaccacgtactcttgtgtcggtgtgatgaagtcgggtcgtgtagaaattcttgctaatgaccaaggtaacagaatcactccttcctacgttagtttcactgaagacgagagactggttggtgatgctgctaagaacttagctgcttctaacccaaaaaacaccatctttgatattaagagattgatcggtatgaagtatgatgccccagaggtccaaagagacttgaagcgtcttccttacactgtcaagagcaagaacggccaacctgtcgtttctgtcgagtacaagggtgaggagaagtctttcactcctgaggagatttccgccatggtcttgggtaagatgaagttgatcgctgaggactacttaggaaagaaagtcactcatgctgtcgttaccgttccagcctacttcaacgacgctcaacgtcaagccactaaggatgccggtctgatcgccggtttgactgttctgagaattgtgaacgagcctaccgccgctgcccttgcttacggtttggacaagactggtgaggaaagacagatcatcgtctacgacttgggtggaggaaccttcgatgtttctctgctttctattgagggtggtgctttcgaggttcttgctaccgccggtgacacccacttgggtggtgaggactttgactacagagttgttcgccacttcgttaagattttcaagaagaagcataacattgacatcagcaacaatgataaggctttaggtaagctgaagagagaggtcgaaaaggccaagcgtactttgtcttcccagatgactaccagaattgagattgactctttcgttgacggtatcgacttctctgagcaactgtctagagctaagtttgaggagatcaacattgaattattcaagaagacactgaaaccagttgaacaagtcctcaaagacgctggtgtcaagaaatctgaaattgatgacattgtcttggttggtggttctaccagaatcccaaaggttcaacaattattggaggattactttgacggaaagaaggcttctaagggaattaacccagatgaagctgtcgcatacggtgctgctgttcaggctggtgttttgtctggtgaggaaggtgtcgatgacatcgtcttgcttgatgtgaaccccctaactctgggtatcgagactactggtggcgttatgactaccttaatcaacagaaacactgctatcccaactaagaaatctcaaattttctccactgctgctgacaaccagccaactgtgttgattcaagtttatgagggtgagagagccttggctaaggacaacaacttgcttggtaaattcgagctgactggtattccaccagctccaagaggtactcctcaagttgaggttacttttgttttagacgctaacggaattttgaaggtttctgccaccgataagggaactggaaaatccgagtccatcaccatcaacaatgatcgtggtagattgtccaaggaggaggttgaccgtatggttgaagaggccgagaagtacgccgctgaggatgctgcactaagagaaaagattgaggctagaaacgctctggagaactacgctcattcccttaggaaccaagttactgatgactctgaaaccgggcttggttctaaattggacgaggacgacaaagagacattgacagatgccatcaaagataccctagagttcttggaagacaacttcgacaccgcaaccaaggaagaattagacgaacaaagagaaaagctttccaagattgcttacccaatcacttctaagctatacggtgctccagagggtggtactccacctggtggtcaaggttttgacgatgatgatggagactttgactacgactatgactatgatcatgatgagttgtaa

SEQ ID NO: 39 - protein, 568 aa, synthetic construct
MDLVKVGQSYVDKIVTDTGIKVLLLDDITSSIISLVSTQSELLNHQVYLIDKLENENRDTIKQLDCVCFLSVSEKTINLLVEELGAPKYKSYKLYFNNVVPNSFLERLAERDDLEMVDKVMELFLDYDILNKNLFSFKQLNIFNSIDAWNQQQFLLTLASLKSLCFSLQTNPIIRYESNSRMCSKLASDLSYEFGQSSKIMEKFPVNDIPPVLLILDRKNDPITPLLNPWTYQSMVHELLGIFNNTVDLTGTPSDLPPDLIKLVLNPSQDPFYAQSLYLNFGDLSDSIKTYVNEYKEKTVKHNSNELTDLNDMKHFLESFPEFKKLSNNISKHMGLITELDRKINENHLWQVSELEQSIAVNDNHNADLQELEKLLTSQEFKIANNLKVKLVCLYAIRYELHPNNQLPKMLSILLQQGVPEFEINTVNRMLKYSGSTKRLNDDSESSIFNQATNNLLQGFKQSHENDNIYMQHIPRLERVISKLVKNKLPTAHYPTLINDFLKKQRPVSDLNGARLQDIIIFFVGGVTYEEARIINNFNLVNKSTRIVIGGTTVHNTNSFMTQVLELE

SEQ ID NO: 40 - DNA, 1707 bp, synthetic construct
  source 1..1707: mol_type = other DNA
atggacttggttaaggttggacaatcctacgtggataaaattgtcacagacacaggcattaaggttcttttattggatgatatcacttcttccataatttccctagtgagcacccaatcagaattgttgaaccatcaggtgtatttgatcgacaagttggagaacgagaatagagatacgataaagcaattggattgtgtgtgtttcctatcagtatcagaaaaaactataaacttgcttgttgaggaattaggtgctcccaaatacaaatcctacaagctctacttcaataatgtagttcccaactcattcttagagaggttggcggagagggacgatttggaaatggtcgataaggtcatggaattgttcctagattacgacattttgaacaagaacttgttttccttcaaacaactgaatattttcaattcaattgatgcttggaatcagcaacagtttctcttgactttagcaagcttgaaatcactctgcttctccttgcaaacgaatcctataatcaggtatgaatctaatagtcgaatgtgttctaagctagcttccgatttgtcatacgaatttgggcaaagttctaaaattatggaaaagttcccggtgaatgatatccctcctgtcctgttaattcttgaccgaaaaaacgacccaatcactccattattaaatccttggacttatcaatctatggtacacgagcttttaggaattttcaataatacggtggatttaacgggaactccttctgatctgcccccagacctaatcaaactggtattgaatccctctcaagatccattttatgctcagtctctatatttgaatttcggagacttgtccgatagtataaaaacatacgtaaacgagtacaaagaaaaaaccgtcaaacacaattctaatgaattgacagatttgaatgatatgaaacactttctggaatcttttccagagttcaaaaaactttcaaacaacatttccaaacacatgggcttgattacagaattagatagaaaaatcaacgaaaatcacttatggcaagtgagtgaattggaacaatccatagctgttaatgacaatcataatgctgaccttcaagaactagaaaagctgttgacatctcaagagttcaagattgccaacaacttaaaagttaaattagtatgtttgtatgccatacgatatgaacttcatcccaacaaccagcttccaaaaatgttgtcaatacttttacagcagggggtgccagagtttgaaataaatacagtcaacaggatgttgaaatactcgggaagtaccaaacgattgaatgatgactctgaatcttcgatatttaaccaggcaacaaataatctactgcaggggttcaaacaaagtcatgaaaacgacaatatttatatgcagcatattccaaggttggaaagagttatcagcaagttagtgaaaaataagctacccacagcgcattatccgactttaatcaatgattttttgaagaagcaacgccctgtttctgatctaaatggagccaggctgcaagatattattattttctttgttggtggagtcacttatgaagaggcccgaataattaacaatttcaatctggtgaacaagtctacgaggatagttatagggggaactacagtacacaacacgaatagttttatgactcaagttctagaattggagtaa

SEQ ID NO: 41 - protein, 645 aa, synthetic construct
MSFTTSLPSLRDRQIATLEKMLHLNEPIVDNGSDIQAELTWKVLILDSRSTAIVSSVLRVNDLLSSGITMHSNIRSKRAALPDVPVIYFVEPNAENINFIIDDLERDQYAHFYINFTSSLNRDLLEEFAKKVATIGKSYKIKQVYDQYLDYIVTEPNLFSLDLVNIYSQLNNPNSLEDEINKVADKISNGIFAAILTMNGIPTIRCCRGGPAELIASKLDQKLRDHVINTKSSASFTNSKLVLILLDRNIDLASMFAHSWIYQCMVSDVFELKRNTIKIPSQKPNESTKEYDIDPKDFFWAANNSLPFPDAVENVENELSRYKADAAELTRKTGVSSLQDIDPNAITDTTDIQLAVKSLPELAFRKSILDMHMKVLASLLQELESKSLDSYFEIEQNYKDPKNQKQFISILNNGNEHTLNDKLRTYIMLYLLTDLPGSFVEECEEYFKKNSAELGSLSYIKRAKEVIKLSNYELSMSIDASHSTTSGLVNEAQKSALFQGLSSKLYGLTDGGSRLTEGVGSLITGLKNLLPDKKQLPITNIVESIMEPSLATQESIKLTDDYLYFDPISTRGVHSKPPKRQQYNNSIVFVVGGGNYLEYQNLQEWVTKTNTSNVNGTKSVIYGSTSIVTANEFLKECSLLGAEAK

SEQ ID NO: 42 - DNA, 1878 bp, synthetic construct
  source 1..1878: mol_type = other DNA
atgcttcatttgaatgagcccattgtggataatggttcagatatacaagcggagttaacatggaaggtactgattctggatagtaggagtactgcaattgtttcttctgttctgcgagttaatgacctgctttcttctggcatcactatgcatagcaatatcagatccaagagagcggctttgccagatgttcctgtcatttactttgttgaacctaatgcggaaaatatcaactttatcattgatgacttggaaagagatcagtacgctcatttttatatcaacttcacttccagtctaaatagggaccttttggaggagtttgctaagaaagtggctacgattggtaagtcctacaagattaaacaggtttatgatcagtacctcgattacattgtcactgaacccaacctgttctctttggacttggttaacatttactcgcagctaaataaccctaactcactggaagatgaaatcaataaagttgctgacaagatttccaatggtatattcgcagcaatcctaactatgaatggtatccctactattagatgttgcagaggaggtccagcagaactaatagcgtccaaactagatcagaagctacgtgatcatgttatcaatacaaagtcatctgcctctttcactaacagtaaattagtgcttatcctgctggatagaaacattgatttggcttccatgtttgctcattcatggatttatcaatgtatggtgagtgatgtttttgagttgaaaagaaatacaatcaaaattccctctcaaaagcccaatgaatctacgaaagaatatgatatcgacccaaaggattttttttgggcagccaacaacagtttgcccttccctgatgctgtagaaaatgtggagaacgaactttctagatacaaagcggatgctgcagagctaactagaaagactggggtttcttctcttcaagatattgatcccaatgcaattactgacaccacagatatacagcttgctgtgaagtctttacctgaattggcttttagaaaaagcatccttgatatgcacatgaaagtacttgcgtctttgctgcaagaactggaatcaaagtcattggattcatactttgaaattgaacaaaactacaaagatcccaaaaaccagaagcagtttatcagtatcctcaacaacgggaatgagcataccttgaacgacaaactgagaacctacatcatgttgtatctgttaacagacctcccagggtcgttcgttgaagaatgtgaagagtatttcaaaaagaactccgctgagcttggttcgttgagttatatcaagcgggcaaaagaggtgatcaagttgtctaattatgagttgtccatgtcaattgatgctagccactcgaccactagtggattggtgaatgaagctcaaaagtctgctttgttccaaggattgtcgtccaagctatatggattaacagatggtggtagtaggcttacagagggggtggggtcattaattactgggttgaaaaacttgctacccgacaagaaacaactgcctattaccaatattgttgaatcgataatggaaccaagtctggccactcaagagtcgataaaactaacggacgattacctatattttgaccctattagcacaagaggagttcactccaaaccacccaaaagacagcaatacaacaattctattgtgtttgttgtaggagggggcaactatttggagtaccaaaatttgcaagaatgggttacgaagaccaatactagcaacgtcaatggcactaagtctgtaatctacggtagtaccagtatcgtgaccgcgaacgagttcttgaaggagtgctccttgctcggtgccgaagcaaaataa

SEQ ID NO: 43 - protein, 161 aa, synthetic construct
MSSFYDLAPLDKKGEPFPFEQLKGKVVLIVNVASKCGFTPQYTELEKLYKDHKDEGLTIVGFPCNQFGHQEPGNDEEIGQFCQLNFGVTFPILKKIDVNGSEADPVYEFLKSKKSGLLGFKGIKWNFEKFLIDKQGNVIERYSSLTKPSSIESKIEELLKK

SEQ ID NO: 44 - DNA, 486 bp, synthetic construct
  source 1..486: mol_type = other DNA
atgtcttcattttatgatctggccccattagataagaaaggcgaaccttttcctttcgaacaattaaaaggcaaagtggtgttgattgtgaatgttgcttctaagtgtgggtttactccacaatataccgagttggaaaagctctacaaagaccacaaggacgagggattgactattgtcggatttccctgtaaccagtttggtcatcaggaaccaggaaatgatgaagaaattggacagttttgccagttgaattttggtgtaactttcccaattctaaaaaagattgatgtcaacggttcggaagctgatcctgtttacgaatttctcaagtcaaaaaagtctggtctgctcggattcaaaggtattaagtggaactttgaaaaattcttgatcgataagcaaggaaacgttattgagagatattcgtccttgactaagccctcatcgatcgagtccaagattgaagaactattaaagaaataa

SEQ ID NO: 45 - protein, 85 aa, synthetic construct
MRFPSIFTAVLFAASSALAAPVNTTTEDETAQIPAEAVIGYSDLEGDFDVAVLPFSNSTNNGLLFINTTIASIAAKEEGVSLEKR

SEQ ID NO: 46 - DNA, 255 bp, synthetic construct
  source 1..255: mol_type = other DNA
atgagatttccttcaatttttactgctgttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaaga

SEQ ID NO: 47 - protein, 6 aa, synthetic construct
KREAEA

SEQ ID NO: 48 - DNA, 18 bp, synthetic construct
  source 1..18: mol_type = other DNA
aagcgagaagccgaagca

SEQ ID NO: 49 - protein, 8 aa, synthetic construct
KREAEAEA

SEQ ID NO: 50 - DNA, 24 bp, synthetic construct
  source 1..24: mol_type = other DNA
aagcgagaagcagaagcagaagcg

SEQ ID NO: 51 - protein, 10 aa, synthetic construct
KREAEAEAEA

SEQ ID NO: 52 - DNA, 30 bp, synthetic construct
  source 1..30: mol_type = other DNA
aagcgagaagcagaagcagaagcagaagcg

SEQ ID NO: 53 - protein, 112 aa, synthetic construct
DKCKKVYENYPVSKCQLANQCNYDCKLDKHARSGECFYDEKRNLQCICDYCEYKREAEADKCKKVYENYPVSKCQLANQCNYDCKLDKHARSGECFYDEKRNLQCICDYCEY

SEQ ID NO: 54 - DNA, 336 bp, synthetic construct
  source 1..336: mol_type = other DNA
gataagtgtaagaaggtatatgagaactatccagttagtaagtgtcaattggccaatcaatgcaactatgactgcaagttggacaagcacgccagaagtggtgagtgcttctacgatgagaagcggaatcttcaatgtatctgcgactactgtgaatacaagcgagaagccgaagcagataagtgtaagaaggtatatgagaactatccagttagtaagtgtcaattggccaatcaatgcaactatgactgcaagttggacaagcacgccagaagtggtgagtgcttctacgatgagaagcggaatcttcaatgtatctgcgactactgtgaatac

SEQ ID NO: 55 - protein, 114 aa, synthetic construct
DKCKKVYENYPVSKCQLANQCNYDCKLDKHARSGECFYDEKRNLQCICDYCEYKREAEAEADKCKKVYENYPVSKCQLANQCNYDCKLDKHARSGECFYDEKRNLQCICDYCEY

SEQ ID NO: 56 - DNA, 342 bp, synthetic construct
  source 1..342: mol_type = other DNA
gataagtgtaagaaggtatatgagaactatccagttagtaagtgtcaattggccaatcaatgcaactatgactgcaagttggacaagcacgccagaagtggtgagtgcttctacgatgagaagcggaatcttcaatgtatctgcgactactgtgaatacaagcgagaagcagaagcagaagcggataagtgtaagaaggtatatgagaactatccagttagtaagtgtcaattggccaatcaatgcaactatgactgcaagttggacaagcacgccagaagtggtgagtgcttctacgatgagaagcggaatcttcaatgtatctgcgactactgtgaatac

SEQ ID NO: 57 - protein, 116 aa, synthetic construct
DKCKKVYENYPVSKCQLANQCNYDCKLDKHARSGECFYDEKRNLQCICDYCEYKREAEAEAEADKCKKVYENYPVSKCQLANQCNYDCKLDKHARSGECFYDEKRNLQCICDYCEY

SEQ ID NO: 58 - DNA, 348 bp, synthetic construct
  source 1..348: mol_type = other DNA
gataagtgtaagaaggtatatgagaactatccagttagtaagtgtcaattggccaatcaatgcaactatgactgcaagttggacaagcacgccagaagtggtgagtgcttctacgatgagaagcggaatcttcaatgtatctgcgactactgtgaatacaagcgagaagcagaagcagaagcagaagcggataagtgtaagaaggtatatgagaactatccagttagtaagtgtcaattggccaatcaatgcaactatgactgcaagttggacaagcacgccagaagtggtgagtgcttctacgatgagaagcggaatcttcaatgtatctgcgactactgtgaatac

(19) United States
(12) Patent Application Publication
(10) Pub. No.: US 2025/0145675 A1
(43) Pub. Date: May 8, 2025

(54) PRODUCTION OF NATURAL PEPTIDE SWEETENER

(71) Applicant: Conagen Inc., Bedford, MA (US)

(72) Inventors: Guohong Mao, Burlington, MA (US); Timothy Joseph Hanly, Waltham, MA (US); Oliver Yu, Lexington, MA (US)

(73) Assignee: Conagen Inc., Bedford, MA (US)

(21) Appl. No.: 18/745,784

(22) Filed: Jun. 17, 2024

Related U.S. Application Data
(63) Continuation of application No. PCT/US2022/081733, filed on Dec. 16, 2022.
(60) Provisional application No. 63/352,351, filed on Jun. 15, 2022.
(60) Provisional application No. 63/291,008, filed on Dec. 17, 2021.

(51) Int. Cl. C07K 14/43; C12N 1/16; C12N 15/81; C12R 1/645

(52) U.S. Cl. CPC C07K 14/43; C12N 1/16; C12N 15/81; C12R 2001/645

(57) ABSTRACT

Provided herein are biosynthetic methods for producing brazzein and related compositions. Uses (e.g., as sweeteners) of the brazzein produced using the methods are also provided.

### RELATED APPLICATIONS

This application is a continuation of International Application No.: PCT/US2022/081733, entitled “PRODUCTION OF NATURAL PEPTIDE SWEETENER”, filed Dec. 16, 2022, which claims the benefit under 35 U.S.C. § 119 (e) to U.S. Provisional Application No. 63/291,008, entitled “PRODUCTION OF NATURAL PEPTIDE SWEETENER”, filed on Dec. 17, 2021, and U.S. Provisional Application No. 63/352,351, entitled “PRODUCTION OF NATURAL PEPTIDE SWEETENER”, filed on Jun. 15, 2022; the contents of each of which are incorporated herein by reference in their entirety.

### FIELD OF THE INVENTION

The contents of the electronic sequence listing (C149770072US02-SEQ-VLJ.xml; Size: 79,937 bytes; and Date of Creation: Jun. 17, 2024) is herein incorporated by reference in its entirety

### FIELD OF THE INVENTION

The field of the invention relates to methods and processes useful in the production of natural peptide sweeteners.

### BACKGROUND

Zero- or low-calorie sweetener or sugar substitutes that can be used in foods and/or beverages to replace or reduce high-calorie sweeteners and/or sugar content are desirable. Brazzein protein was first isolated from the fruit of Pentadiplandra brazzeana Baillon and has been reported to be multiple times sweeter than sucrose and may be suitable for use as sweeteners.

### SUMMARY

The present disclosure, in some aspects, provide methods of producing brazzein, the method comprising culturing a recombinant yeast cell comprising a polynucleotide encoding a polypeptide comprising tandem repeats of brazzein.

In some embodiments, each repeat of brazzein comprises the amino acid sequence of SEQ ID NO: 1. In some embodiments, each brazzein in the tandem repeats is separated by a spacer. In some embodiments, the spacer comprises a protease cleavage site for a yeast protease. In some embodiments, the spacer comprises the amino acid sequence of any one of SEQ ID NOs: 3, 5, 7, 47, 49, and 51. In some embodiments, the polypeptide comprises 2-8 repeats of brazzein. In some embodiments, the polypeptide comprises an amino acid sequence at least 80% (e.g., at least 80%, at least 85%, at least 90%, at least 95%, at least 99%) identical to the amino acid sequence of any one of SEQ ID NOs: 9, 11, 13, 15, 17, 19, 53, 55, and 57.

In some embodiments, the polypeptide comprises the amino acid sequence of any one of SEQ ID NOs: 9, 11, 13, 15, 17, 19, 53, 55, and 57. In some embodiments, the polypeptide further comprises an N-terminal signal peptide. In some embodiments, the signal peptide is a yeast alpha mating factor signal peptide. In some embodiments, the yeast alpha mating factor signal peptide comprises the amino acid sequence of SEQ ID NO: 45.

In some embodiments, the polynucleotide is operably linked to a promoter. In some embodiments, the promoter is an AOX1 promoter. In some embodiments, the polynucleotide is operably linked to a transcription terminator. In some embodiments, the transcription terminator is an AOX1 terminator.

In some embodiments, the polynucleotide is provided on a vector, optionally wherein the vector is a plasmid. In some embodiments, the polynucleotide is integrated into the genome of the recombinant yeast cell. In some embodiments, the polynucleotide is integrated into a HIS4 locus of the genome of the recombinant yeast cell.

In some embodiments, the recombinant yeast cell further comprises one or more polynucleotides encoding one or more chaperones selected from: PD1, PDI1, HAC1, ERO1, ERO2, ERV1, ERV2, ERO1, KAR2, SEC1, SLY1, and GPX1. In some embodiments, the recombinant yeast cell further comprises a polynucleotide encoding PD1, a polynucleotide encoding HAC1, one or more polynucleotides encoding PD1, ERO1, and ERV2, or one or more polynucleotides encoding PD1, ECR1, and ERO1. In some embodiments, the one or more polynucleotides encoding the one or more chaperones are provided on one or more vectors. In some embodiments, the one or more polynucleotides encoding the one or more chaperones are integrated into the genome of the recombinant yeast cell. In some embodiments, the one or more polynucleotides are integrated into a HIS4 locus of the genome of the recombinant yeast cell.

In some embodiments, the recombinant yeast cell further comprises one or more polynucleotides encoding one or more proteases selected from KEX1, KEX2, and Ste13. In some embodiments, the one or more polynucleotides encoding the one or more proteases are provided on one or more vectors. In some embodiments, the one or more polynucleotides encoding the one or more proteases are integrated into the genome of the recombinant yeast cell. In some embodiments, the one or more polynucleotides are integrated into a HIS4 locus of the genome of the recombinant yeast cell.

In some embodiments, the yeast cell is a Pichia pastoris cell.

In some embodiments, the method further comprises isolating the brazzein.

Uses of brazzein produced using the method described herein as a sweetener are also provided.

In some aspects, the present disclosure provide compositions or consumable products comprising the brazzein produced using the method described herein. In some embodiments, the composition or the consumable product further comprises a second sweetener. In some embodiments, the second sweetener is a rebaudioside. In some embodiments, the composition or the consumable product at least one additive is selected from the group consisting of a carbohydrate, a polyol, an amino acid or salt thereof, a polyamino acid or salt thereof, a sugar acid or salt thereof, a nucleotide, an organic acid, an inorganic acid, an organic salt, an organic acid salt, an organic base salt, an inorganic salt, a bitter compound, a flavorant, a flavoring ingredient, an astringent compound, a protein, a protein hydrolysate, a surfactant, an emulsifier, a flavonoids, an alcohol, a polymer, and combinations thereof.

In some embodiments, the consumable product is selected from: a food product, a beverage product, a nutraceutical, a pharmaceutical, a dietary supplement, a dental hygienic composition, an edible gel composition, a cosmetic product and a tabletop flavoring. In some embodiments, the beverage product is selected from the group consisting of a carbonated beverage product and a non-carbonated beverage product. In some embodiments, the beverage product is selected from the group consisting of a soft drink, a fountain beverage, a frozen beverage; a ready-to-drink beverage; a frozen and ready-to-drink beverage, coffee, tea, a dairy beverage, a powdered soft drink, a liquid concentrate, flavored water, enhanced water, fruit juice, a fruit juice flavored drink, a sport drink, and an energy drink.

Further provided herein are polynucleotides comprising the nucleotide sequence of any one of SEQ ID NOs: 2, 10, 12, 14, 16, 18, 20, 24, 28, 30, 32, 54, 56, and 58.

Also provided herein are polypeptides comprising an amino acid sequence that is at least 80% (e.g., at least 80%, at least 85%, at least 90%, at least 95%, at least 99%) identical to any one of SEQ ID NOs: 9, 11, 13, 15, 17, and 19. In some embodiments, the polypeptide comprises the amino acid sequence of any one of SEQ ID NOs: 9, 11, 13, 15, 17, 19, 53, 55, and 57.

Further provided herein are recombinant yeast cells comprising a polynucleotide encoding a polypeptide comprising tandem repeats of brazzein. In some embodiments, the recombinant yeast cell comprises a polypeptide comprising an amino acid sequence that is at least 80% identical to any one of SEQ ID NOs: 9, 11, 13, 15, 17, and 19, 53, 55, and 57. In some embodiments, the polypeptide comprises the amino acid sequence of any one of SEQ ID NOs: 9, 11, 13, 15, 17, 19, 53, 55, and 57.

In some embodiments, the recombinant yeast cell comprises a polynucleotide comprising the nucleotide sequence of any one of SEQ ID NOs: 2, 10, 12, 14, 16, 18, 20, 54, 56, and 58. In some embodiments, the recombinant yeast cell comprises one or more polynucleotides comprising the nucleotide sequence of any one of SEQ ID NOs: 24, 28, 30, and 32.

While the disclosure is susceptible to various modifications and alternative forms, specific embodiments thereof are shown by way of example in the drawing and will herein be described in detail. It should be understood, however, that the drawings and detailed description presented herein are not intended to limit the disclosure to the particular embodiments disclosed, but on the contrary, the intention is to cover all modifications, equivalents, and alternatives falling within the spirit and scope of the present disclosure as defined by the appended claims.

Other features and advantages of this invention will become apparent in the following detailed description of preferred embodiments of this invention, taken with reference to the accompanying drawings.

### BRIEF DESCRIPTION OF THE DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIG. 1 provides a schematic of the plasmid map of pHKA-Brax 1. The plasmid is composed of 7,895 bp, and contains an AOX1 promoter, and an alpha factor signal peptide fused to Brazzein.

FIG. 2 provides a schematic of the plasmid map of pHKA-Brax8. The plasmid is composed of 20,334 bp, and contains 8 copies of the AOX1 promoter, and an alpha factor signal peptide fused to Brazzein.

FIG. 3 provides an SDS-PAGE analysis of medium samples from induced culture.

Legend: M: standard ladder; 1. Brax8 sample; 2. Brax1 sample.

FIGS. 4A-4B provides an HPLC analysis of brazzein standard and Brax8 sample. In panel (FIG. 4A) is the Brazzein standard; and in panel (FIG. 4B) is the Brax8 medium sample. Arrows indicate peaks corresponding to brazzein.

FIG. 5 provides a schematic of the plasmid map of pHKA-Bra-KRx2. The plasmid is composed of 8,060 bp, and contains the AOX1 promoter, an alpha factor signal peptide, Brazzein, a KR sequence and a second copy of Brazzein.

FIG. 6 provides a schematic of the plasmid map of pHKA-Bra-KRx8. The plasmid is composed of 9,050 bp.

FIG. 7 provides a schematic of the plasmid map of pHKA-Bra-KREAx2. The plasmid is composed of 8,066 bp, and contains the AOX1 promoter, an alpha factor signal peptide, Brazzein, a KREA sequence and a second copy of Brazzein.

FIG. 8 provides a schematic of the plasmid map of pHKA-Bra-KREAx8. The plasmid is composed of 9,092 bp.

FIG. 9 provides a schematic of the plasmid map of pHKA-Bra-KR (EA) 5×2. The plasmid is composed of 8,090 bp, and contains the AOX1 promoter, an alpha factor signal peptide, Brazzein, a KR (EA) 5 sequence and a second copy of Brazzein.

FIG. 10 provides a schematic of the plasmid map of pHKA-Bra-KR (EA) 5×8. The plasmid is composed of 9,260 bp.

FIG. 11 demonstrates that co-expression of AtPD1 in Brax8 strains 2, 7 and 18 increased brazzein production. Brax8: parent strain, 2,7, 18: individual colonies from transformation of pPICZ-AtPD1 into Brax8 strain.

FIG. 12 demonstrates that co-expression of HAC1 in Brax8 strains increased brazzein production relative to Brax8 parent strain. Brax8: parent strain, 8,18,19: individual colonies from transformation of pPICZ-HAC1 into Brax8 strain.

FIG. 13 demonstrates that co-expression of PpPDI/PpERO1/PpERV2 in Brax8 strains increased brazzein production relative to Brax8 parent strain. Brax8: parent strain, 7, 19, 20: individual colonies from transformation of pPICZ-PpPDI/PpERO1/PpERV2 into Brax8 strain.

FIG. 14 demonstrates that co-expression of AtPD1/AtERV1/AtERO1 in Brax8 strains increased brazzein production relative to Brax 8 parent strain. Brax8: parent strain, 10, 12: individual colonies from transformation of pPICZ-AtPD1/AtERV/AtERO1 into Brax8 strain.

FIG. 15 provides an SDS-PAGE analysis fermentation samples. Lane 1 is the Brazzein standard; Lane M: protein ladder; Lane 2 and 3: samples before methanol induction. 17 hr and 21 hr; 4-10: samples after methanol induction, 42, 46, 49, 66, 73, 92 and 120 hr.

FIG. 16 provides a schematic of the plasmid map of pHKA-Bra-KREAEAx2. The plasmid is composed of 8,072 bp.

FIG. 17 provides a schematic of the plasmid of pHKA-Bra-KR (EA) 3×2. The plasmid is composed of 8,078 bp.

FIG. 18 provides a schematic of the plasmid of pHKA-Bra-KR (EA) 4×2. The plasmid is composed of 8,084 bp.

FIG. 19 provides an SDS-PAGE analysis of media from induced cell cultures. M: protein standard ladder; 1-2: samples from two Bra-KREAx2 strain; 3-4: samples from two individual Bra-KREAEAx2 strains; 5-6: samples from two individual Bra-KR (EA) 3×2 strains; 7-8: samples from two individual Bra-KR (EA) 4×2 strains; 9-10: samples from two individual Bra-KR (EA) 5×2 strains. Arrow shows brazzein.

FIGS. 20A-20B provides an LC-MS analysis of brazzein standard and samples. FIG. 20A: brazzein standard; FIG. 20B: sample number 4 from FIG. 19.

### DEFINITIONS

“Cellular system” is any cells that provide for the expression of ectopic proteins. It includes bacteria, yeast, plant cells and animal cells. It may include prokaryotic or eukaryotic host cells which are modified to express a recombinant protein and cultivated in an appropriate culture medium. It also includes the in vitro expression of proteins based on cellular components, such as ribosomes.

“Coding sequence” is to be given its ordinary and customary meaning to a person of ordinary skill in the art, and is used without limitation to refer to a DNA sequence that encodes for a specific amino acid sequence.

“Growing the Cellular System”. Growing includes providing an appropriate medium that would allow cells to multiply and divide, to form a cell culture. It also includes providing resources so that cells or cellular components can translate and make recombinant proteins.

“Protein Expression”. Protein production can occur after gene expression. It consists of the stages after DNA has been transcribed to messenger RNA (mRNA). The mRNA is then translated into polypeptide chains, which are ultimately folded into proteins. DNA or RNA may be present in the cells through transfection—a process of deliberately introducing nucleic acids into cells. The term is often used for non-viral methods in eukaryotic cells. It may also refer to other methods and cell types, although other terms are preferred: “transformation” is more often used to describe non-viral DNA transfer in bacteria, non-animal eukaryotic cells, including plant cells. In animal cells, transfection is the preferred term as transformation is also used to refer to progression to a cancerous state (carcinogenesis) in these cells.

Transduction is often used to describe virus-mediated DNA transfer. Transformation, transduction, and viral infection are included under the definition of transfection for this application.

“Yeast”. According to the current disclosure a yeast are eukaryotic, single-celled microorganisms classified as members of the fungus kingdom. Yeasts are unicellular organisms which are believed to have evolved from multicellular ancestors.

As used herein, the singular forms “a, an” and “the” include plural references unless the content clearly dictates otherwise.

To the extent that the term “include,” “have,” or the like is used in the description or the claims, such term is intended to be inclusive in a manner similar to the term “comprise” as “comprise” is interpreted when employed as a transitional word in a claim.

The word “exemplary” is used herein to mean “serving as an example, instance, or illustration.” Any embodiment described herein as “exemplary” is not necessarily to be construed as preferred or advantageous over other embodiments.

The term “complementary” is to be given its ordinary and customary meaning to a person of ordinary skill in the art and is used without limitation to describe the relationship between nucleotide bases that are capable to hybridizing to one another. For example, with respect to DNA, adenosine is complementary to thymine and cytosine is complementary to guanine. Accordingly, the subject technology also includes isolated nucleic acid fragments that are complementary to the complete sequences as reported in the accompanying Sequence Listing as well as those substantially similar nucleic acid sequences.

The terms “nucleic acid” and “nucleotide” are to be given their respective ordinary and customary meanings to a person of ordinary skill in the art and are used without limitation to refer to deoxyribonucleotides or ribonucleotides and polymers thereof in either single- or double-stranded form. Unless specifically limited, the term encompasses nucleic acids containing known analogues of natural nucleotides that have similar binding properties as the reference nucleic acid and are metabolized in a manner similar to naturally-occurring nucleotides. Unless otherwise indicated, a particular nucleic acid sequence also implicitly encompasses conservatively modified or degenerate variants thereof (e.g., degenerate codon substitutions) and complementary sequences, as well as the sequence explicitly indicated.

The term “isolated” is to be given its ordinary and customary meaning to a person of ordinary skill in the art, and when used in the context of an isolated nucleic acid or an isolated polypeptide, is used without limitation to refer to a nucleic acid or polypeptide that, by the hand of man, exists apart from its native environment and is therefore not a product of nature. An isolated nucleic acid or polypeptide can exist in a purified form or can exist in a non-native environment such as, for example, in a transgenic host cell.

The terms “incubating” and “incubation” as used herein means a process of mixing two or more chemical or biological entities (such as a chemical compound and an enzyme) and allowing them to interact under conditions favorable for producing a 8-lactone composition.

The term “degenerate variant” refers to a nucleic acid sequence having a residue sequence that differs from a reference nucleic acid sequence by one or more degenerate codon substitutions. Degenerate codon substitutions can be achieved by generating sequences in which the third position of one or more selected (or all) codons is substituted with mixed base and/or deoxyinosine residues. A nucleic acid sequence and all of its degenerate variants will express the same amino acid or polypeptide.

The terms “polypeptide,” “protein,” and “peptide” are to be given their respective ordinary and customary meanings to a person of ordinary skill in the art; the three terms are sometimes used interchangeably, and are used without limitation to refer to a polymer of amino acids, or amino acid analogs, regardless of its size or function. Although “protein” is often used in reference to relatively large polypeptides, and “peptide” is often used in reference to small polypeptides, usage of these terms in the art overlaps and varies. The term “polypeptide” as used herein refers to peptides, polypeptides, and proteins, unless otherwise noted. The terms “protein,” “polypeptide,” and “peptide” are used interchangeably herein when referring to a polyaminoacid product. Thus, exemplary polypeptides include polyaminoacid products, naturally occurring proteins, homologs, orthologs, paralogs, fragments and other equivalents, variants, and analogs of the foregoing.

The terms “polypeptide fragment” and “fragment,” when used in reference to a reference polypeptide, are to be given their ordinary and customary meanings to a person of ordinary skill in the art and are used without limitation to refer to a polypeptide in which amino acid residues are deleted as compared to the reference polypeptide itself, but where the remaining amino acid sequence is usually identical to the corresponding positions in the reference polypeptide. Such deletions can occur at the amino-terminus or carboxy-terminus of the reference polypeptide, or alternatively both.

The term “functional fragment” of a polypeptide or protein refers to a peptide fragment that is a portion of the full-length polypeptide or protein, and has substantially the same biological activity, or carries out substantially the same function as the full-length polypeptide or protein (e.g., carrying out the same enzymatic reaction).

The terms “variant polypeptide,” “modified amino acid sequence” or “modified polypeptide,” which are used interchangeably, refer to an amino acid sequence that is different from the reference polypeptide by one or more amino acids, e.g., by one or more amino acid substitutions, deletions, and/or additions. In an aspect, a variant is a “functional variant” which retains some or all of the ability of the reference polypeptide.

The term “functional variant” further includes conservatively substituted variants. The term “conservatively substituted variant” refers to a peptide having an amino acid sequence that differs from a reference peptide by one or more conservative amino acid substitutions and maintains some or all of the activity of the reference peptide. A “conservative amino acid substitution” is a substitution of an amino acid residue with a functionally similar residue. Examples of conservative substitutions include the substitution of one non-polar (hydrophobic) residue such as isoleucine, valine, leucine or methionine for another; the substitution of one charged or polar (hydrophilic) residue for another such as between arginine and lysine, between glutamine and asparagine, between threonine and serine; the substitution of one basic residue such as lysine or arginine for another; or the substitution of one acidic residue, such as aspartic acid or glutamic acid for another; or the substitution of one aromatic residue, such as phenylalanine, tyrosine, or tryptophan for another. Such substitutions are expected to have little or no effect on the apparent molecular weight or isoelectric point of the protein or polypeptide. The phrase “conservatively substituted variant” also includes peptides wherein a residue is replaced with a chemically-derivatized residue, provided that the resulting peptide maintains some or all of the activity of the reference peptide as described herein.

The term “variant,” in connection with the polypeptides of the subject technology, further includes a functionally active polypeptide having an amino acid sequence at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, and even 100% identical to the amino acid sequence of a reference polypeptide.

The term “homologous” in all its grammatical forms and spelling variations refers to the relationship between polynucleotides or polypeptides that possess a “common evolutionary origin,” including polynucleotides or polypeptides from super-families and homologous polynucleotides or proteins from different species (Reeck et al., CELL 50:667, 1987). Such polynucleotides or polypeptides have sequence homology, as reflected by their sequence similarity, whether in terms of percent identity or the presence of specific amino acids or motifs at conserved positions. For example, two homologous polypeptides can have amino acid sequences that are at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 900 at least 91%, at least 92%, at least 93%, at least 94%, at least 95%, at least 96%, at least 97%, at least 98%, at least 99%, and even 100% identical.

“Suitable regulatory sequences” is to be given its ordinary and customary meaning to a person of ordinary skill in the art, and is used without limitation to refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include promoters, translation leader sequences, introns, and polyadenylation recognition sequences.

“Promoter” is to be given its ordinary and customary meaning to a person of ordinary skill in the art, and is used without limitation to refer to a DNA sequence capable of controlling the expression of a coding sequence or functional RNA. In general, a coding sequence is located 3′ to a promoter sequence. Promoters may be derived in their entirety from a native gene, or be composed of different elements derived from different promoters found in nature, or even comprise synthetic DNA segments. It is understood by those skilled in the art that different promoters may direct the expression of a gene in different tissues or cell types, or at different stages of development, or in response to different environmental conditions. Promoters which cause a gene to be expressed in most cell types at most times, are commonly referred to as “constitutive promoters.” It is further recognized that since in most cases the exact boundaries of regulatory sequences have not been completely defined, DNA fragments of different lengths may have identical promoter activity.

The term “operably linked” refers to the association of nucleic acid sequences on a single nucleic acid fragment so that the function of one is affected by the other. For example, a promoter is operably linked with a coding sequence when it is capable of affecting the expression of that coding sequence (i.e., that the coding sequence is under the transcriptional control of the promoter). Coding sequences can be operably linked to regulatory sequences in sense or antisense orientation.

The term “expression” as used herein, is to be given its ordinary and customary meaning to a person of ordinary skill in the art, and is used without limitation to refer to the transcription and stable accumulation of sense (mRNA) or antisense RNA derived from the nucleic acid fragment of the subject technology. “Over-expression” refers to the production of a gene product in transgenic or recombinant organisms that exceeds levels of production in normal or non-transformed organisms.

“Transformation” is to be given its ordinary and customary meaning to a person of reasonable skill in the field, and is used without limitation to refer to the transfer of a polynucleotide into a target cell for further expression by that cell. The transferred polynucleotide can be incorporated into the genome or chromosomal DNA of a target cell, resulting in genetically stable inheritance, or it can replicate independent of the host chromosomal DNA. Host organisms containing the transformed nucleic acid fragments are referred to as “transgenic” or “recombinant” or “transformed” organisms.

The terms “transformed,” “transgenic,” and “recombinant,” when used herein in connection with host cells, are to be given their respective ordinary and customary meanings to a person of ordinary skill in the art, and are used without limitation to refer to a cell of a host organism, such as a plant or microbial cell, into which a heterologous nucleic acid molecule has been introduced. The nucleic acid molecule can be stably integrated into the genome of the host cell, or the nucleic acid molecule can be present as an extrachromosomal molecule. Such an extrachromosomal molecule can be auto-replicating. Transformed cells, tissues, or subjects are understood to encompass not only the end product of a transformation process, but also transgenic progeny thereof.

The terms “recombinant,” “heterologous,” and “exogenous,” when used herein in connection with polynucleotides, are to be given their ordinary and customary meanings to a person of ordinary skill in the art, and are used without limitation to refer to a polynucleotide (e.g., a DNA sequence or a gene) that originates from a source foreign to the particular host cell or, if from the same source, is modified from its original form. Thus, a heterologous gene in a host cell includes a gene that is endogenous to the particular host cell but has been modified through, for example, the use of site-directed mutagenesis or other recombinant techniques. The terms also include non-naturally occurring multiple copies of a naturally occurring DNA sequence. Thus, the terms refer to a DNA segment that is foreign or heterologous to the cell, or homologous to the cell but in a position or form within the host cell in which the element is not ordinarily found.

Similarly, the terms “recombinant,” “heterologous,” and “exogenous,” when used herein in connection with a polypeptide or amino acid sequence, means a polypeptide or amino acid sequence that originates from a source foreign to the particular host cell or, if from the same source, is modified from its original form. Thus, recombinant DNA segments can be expressed in a host cell to produce a recombinant polypeptide.

The terms “plasmid,” “vector,” and “cassette” are to be given their respective ordinary and customary meanings to a person of ordinary skill in the art and are used without limitation to refer to an extra chromosomal element often carrying genes which are not part of the central metabolism of the cell, and usually in the form of circular double-stranded DNA molecules. Such elements may be autonomously replicating sequences, genome integrating sequences, phage or nucleotide sequences, linear or circular, of a single- or double-stranded DNA or

RNA, derived from any source, in which a number of nucleotide sequences have been joined or recombined into a unique construction which is capable of introducing a promoter fragment and DNA sequence for a selected gene product along with appropriate 3′ untranslated sequence into a cell. “Transformation cassette” refers to a specific vector containing a foreign gene and having elements in addition to the foreign gene that facilitate transformation of a particular host cell. “Expression cassette” refers to a specific vector containing a foreign gene and having elements in addition to the foreign gene that allow for enhanced expression of that gene in a foreign host.

Unless defined otherwise, all technical and scientific terms used herein have the same meaning as commonly understood by one of ordinary skill in the art to which the disclosure belongs. Although any methods and materials similar to or equivalent to those described herein may be used in the practice or testing of the present disclosure, the preferred materials and methods are described below.

### DETAILED DESCRIPTION

Brazzein is natural peptide sweetener which was first isolated from the fruit of Pentadiplandra brazzeana Baillon found in West Africa. Brazzein is 500-2000 times sweeter than sucrose on a weight basis. There are three forms of brazzein identified in fruit. The major form brazzein has pyroglutamic acid at N-terminus has 54 amino acid residues (pyrE-bra). The minor form brazzein (53 aa) has identical amino acid sequence except no N-terminal pyroglutamic acid (des-pyrE-bra). PyrE-bra is 500 times sweeter than 10% sucrose solution and des-pyrE-bra is twice sweeter than pyre-bra. Brazzein is heat stable and its sweet taste remains after incubation at 98° C. for 2 h and at 80° C. for 4.5 h in the pH range of 2.5-8. Brazzein has four intramolecular disulfide bonds which is related to heat-stable of brazzein.

The present disclosure, in some aspects, provide methods of producing brazzein, in which multiple strategies were employed to increase brazzein folding, secretion and/or production in engineered yeast cells (e.g., engineered Pichia cells).

In some embodiments, a method of producing brazzein described herein comprises culturing a recombinant yeast cell comprising a polynucleotide encoding a polypeptide comprising tandem repeats of brazzein. In some embodiments, the polynucleotide is provided on a vector (e.g., a plasmid such as an expression plasmid). In some embodiments, the plasmid is a high copy plasmid (e.g., for high-level expression of the polypeptide comprising the tandem repeats of brazzein). In some embodiments, the polynucleotide is integrated into the genome of the recombinant yeast cell. For example, in some embodiments, the polynucleotide is integrated into a HIS4 locus of the genome of the recombinant yeast cell.

In some embodiments, each repeat of brazzein comprises an amino acid sequence that is at least 70% (e.g., at least 70%, at least 75%, at least 80%, at least 85%, at least 90%, at least 99%, or at least 99%) identical to the amino acid sequence of SEQ ID NO: 1. In some embodiments, each repeat of brazzein comprises the amino acid sequence of SEQ ID NO: 1. In some embodiments, each polypeptide comprises at least 2 (e.g., at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20) repeats of brazzein. In some embodiments, each polypeptide comprises 2-20 (e.g., 2-20, 2-15, 2-10, 2-5, 5-20, 5-15, 5-10, 10-20, 10-15, or 15-20) repeats of brazzein. In some embodiments, each polypeptide comprises 2, 3, 4, 5, 6, 7, 8, 9, 10, 11, 12, 13, 14, 15, 16, 17, 18, 19, or 20 repeats of brazzein. In some embodiments, each polypeptide comprises 8 repeats of brazzein.

In some embodiments, in the polypeptide comprising tandem repeats of brazzein, each brazzein repeat is separated by a spacer. In some embodiments, the spacer is cleaved by a protease (e.g., cleaved in vivo by a protease in the yeast cell). As such, in some embodiments, each spacer between the brazzein repeats comprises a protease cleavage site for a yeast protease. In some embodiments, each spacer comprises the amino acid sequence of any one of SEQ ID NOs: 3, 5 or 7.

In some embodiments, the polypeptide comprises an amino acid sequence least 70% (e.g., at least 70%, at least 75%, at least 80%, at least 85%, at least 90%, at least 99%, or at least 99%) identical to the amino acid sequence of any one of SEQ ID NOs: 9, 11, 13, 15, 17, 19, 53, 55, and 57. In some embodiments, the polypeptide comprises the amino acid sequence of any one of SEQ ID NOs: 9, 11, 13, 15, 17, and 19, 53, 55, and 57. In some embodiments, the polynucleotide comprises a nucleotide sequence at least 70% (e.g., at least 70%, at least 75%, at least 80%, at least 85%, at least 90%, at least 99%, or at least 99%) identical to the nucleotide sequence of any one of SEQ ID NOs: 10, 12, 14, 16, 18, 20, 54, 56, and 58. In some embodiments, the polynucleotide comprises the nucleotide sequence of any one of SEQ ID NOs: 10, 12, 14, 16, 18, 20, 54, 56, and 58.

In some embodiments, the polypeptide further comprises an N-terminal signal peptide. A “signal peptide” refers to a short peptide present at the N-terminus of a protein destined to be secreted from a cell. In some embodiments, a signal peptide comprises a stretch of hydrophobic amino acid residues that facilitate the translocation of a newly synthesized peptide or protein to the cell membrane for subsequent secretion through the cell membrane. In some embodiments, a signal peptide is 5-20 (e.g., 5, 6, 7, 8, 9, 10, 11, 12, 13, 14, 15, 16, 17, 18, 19, or 20) amino acids in length. A protein with a signal peptide can be encapsulated in a secretory vesicle and trafficked to the cell membrane via the secretory pathway. The mechanism by which a newly synthesized peptide or protein comprising a signal peptide is secreted from the cell will be known by a person having ordinary skill in the art.

In some embodiments, the signal peptide is a yeast alpha mating factor signal peptide. In some embodiments, the yeast alpha mating factor signal peptide comprises the amino acid sequence of SEQ ID NO: 45.

In some embodiments, the polynucleotide encoding the polypeptide comprising the tandem repeats of brazzein is operably linked to a promoter. In some embodiments, the promoter is a constitutive promoter (e.g., a constitutive promoter in yeast). In some embodiments, the promoter comprises an AOX1 promoter (e.g., a yeast AOX1 promoter). In some embodiments, the polynucleotide encoding the polypeptide comprising the tandem repeats of brazzein is operably linked to a transcription terminator. In some embodiments, the transcription terminator is an AOX1 terminator (e.g., a yeast AOX1 terminator).

In some embodiments, the method described herein comprising co-expressing the polypeptide comprising the tandem repeats of brazzein with one or more chaperones to facilitate intramolecular disulfide bond formation, folding, and/or secretion. In some embodiments, the one or more chaperones are selected from: PD1, PDI1, HAC1, ERO1, ERO2, ERV1, ERV2, ERO1, KAR2, SEC1, SLY1, and GPX1. Chaperones of the same yeast strain as the yeast recombinant cell used for expression of the polypeptide may be used. Heterologous chaperones from other yeast strains may also be used. Non-limiting examples of chaperones and their Genbank accession numbers are provided in Table 3. As such, in some embodiments, the yeast recombinant cell used in the methods described herein further comprises one or more polynucleotides encoding one or more chaperones selected from PD1, PDI1, HAC1, ERO1, ERO2, ERV1, ERV2, ERO1, KAR2, SEC1, SLY1, and GPX1.

In some embodiments, the PDI comprises an amino acid sequence at least 70% (e.g., at least 70%, at least 75%, at least 80%, at least 85%, at least 90%, at least 99%, or at least 99%) identical to the amino acid sequence of SEQ ID NO: 21. In some embodiments, the PDI comprises the amino acid sequence of SEQ ID NO: 21. In some embodiments, the polynucleotide encoding the PDI comprises a nucleotide sequence at least 70% (e.g., at least 70%, at least 75%, at least 80%, at least 85%, at least 90%, at least 99%, or at least 99%) identical to the nucleotide sequence of SEQ ID NO: 22. In some embodiments, the polynucleotide encoding the PDI comprises the nucleotide sequence of SEQ ID NO: 22.

In some embodiments, the PDI1 comprises an amino acid sequence at least 70% (e.g., at least 70%, at least 75%, at least 80%, at least 85%, at least 90%, at least 99%, or at least 99%) identical to the amino acid sequence of SEQ ID NO: 23. In some embodiments, the PDI1 comprises the amino acid sequence of SEQ ID NO: 23. In some embodiments, the polynucleotide encoding the PDI1 comprises a nucleotide sequence at least 70% (e.g., at least 70%, at least 75%, at least 80%, at least 85%, at least 90%, at least 99%, or at least 99%) identical to the nucleotide sequence of SEQ ID NO: 24. In some embodiments, the polynucleotide encoding the PDI1 comprises the nucleotide sequence of SEQ ID NO: 24.

In some embodiments, the HAC1 comprises an amino acid sequence at least 70% (e.g., at least 70%, at least 75%, at least 80%, at least 85%, at least 90%, at least 99%, or at least 99%) identical to the amino acid sequence of SEQ ID NO: 25. In some embodiments, the HAC1 comprises the amino acid sequence of SEQ ID NO: 25. In some embodiments, the polynucleotide encoding the HAC1 comprises a nucleotide sequence at least 70% (e.g., at least 70%, at least 75%, at least 80%, at least 85%, at least 90%, at least 99%, or at least 99%) identical to the nucleotide sequence of SEQ ID NO: 26. In some embodiments, the polynucleotide encoding the HAC1 comprises the nucleotide sequence of SEQ ID NO: 26.

In some embodiments, the ERO1 comprises an amino acid sequence at least 70% (e.g., at least 70%, at least 75%, at least 80%, at least 85%, at least 90%, at least 99%, or at least 99%) identical to the amino acid sequence of SEQ ID NO: 27 or SEQ ID NO: 35. In some embodiments, the ERO1 comprises the amino acid sequence of SEQ ID NO: 27 or SEQ ID NO: 35. In some embodiments, the polynucleotide encoding the ERO1 comprises a nucleotide sequence at least 70% (e.g., at least 70%, at least 75%, at least 80%, at least 85%, at least 90%, at least 99%, or at least 99%) identical to the nucleotide sequence of SEQ ID NO: 28 or SEQ ID NO: 36. In some embodiments, the polynucleotide encoding the ERO1 comprises the nucleotide sequence of SEQ ID NO: 28 or SEQ ID NO: 36.

In some embodiments, the ERO2 comprises an amino acid sequence at least 70% (e.g., at least 70%, at least 75%, at least 80%, at least 85%, at least 90%, at least 99%, or at least 99%) identical to the amino acid sequence of SEQ ID NO: 29. In some embodiments, the ERO2 comprises the amino acid sequence of SEQ ID NO: 29. In some embodiments, the polynucleotide encoding the ERO2 comprises a nucleotide sequence at least 70% (e.g., at least 70%, at least 75%, at least 80%, at least 85%, at least 90%, at least 99%, or at least 99%) identical to the nucleotide sequence of SEQ ID NO: 30. In some embodiments, the polynucleotide encoding the ERO2 comprises the nucleotide sequence of SEQ ID NO: 30.

In some embodiments, the ERV1 comprises an amino acid sequence at least 70% (e.g., at least 70%, at least 75%, at least 80%, at least 85%, at least 90%, at least 99%, or at least 99%) identical to the amino acid sequence of SEQ ID NO: 31. In some embodiments, the ERV1 comprises the amino acid sequence of SEQ ID NO: 31. In some embodiments, the polynucleotide encoding the ERV1 comprises a nucleotide sequence at least 70% (e.g., at least 70%, at least 75%, at least 80%, at least 85%, at least 90%, at least 99%, or at least 99%) identical to the nucleotide sequence of SEQ ID NO: 32. In some embodiments, the polynucleotide encoding the ERV1 comprises the nucleotide sequence of SEQ ID NO: 32.

In some embodiments, the ERV2 comprises an amino acid sequence at least 70% (e.g., at least 70%, at least 75%, at least 80%, at least 85%, at least 90%, at least 99%, or at least 99%) identical to the amino acid sequence of SEQ ID NO: 33. In some embodiments, the ERV2 comprises the amino acid sequence of SEQ ID NO: 33. In some embodiments, the polynucleotide encoding the ERV2 comprises a nucleotide sequence at least 70% (e.g., at least 70%, at least 75%, at least 80%, at least 85%, at least 90%, at least 99%, or at least 99%) identical to the nucleotide sequence of SEQ ID NO: 34. In some embodiments, the polynucleotide encoding the ERV2 comprises the nucleotide sequence of SEQ ID NO: 34.

In some embodiments, the KAR2 comprises an amino acid sequence at least 70% (e.g., at least 70%, at least 75%, at least 80%, at least 85%, at least 90%, at least 99%, or at least 99%) identical to the amino acid sequence of SEQ ID NO: 37. In some embodiments, the KAR2 comprises the amino acid sequence of SEQ ID NO: 37. In some embodiments, the polynucleotide encoding the KAR2 comprises a nucleotide sequence at least 70% (e.g., at least 70%, at least 75%, at least 80%, at least 85%, at least 90%, at least 99%, or at least 99%) identical to the nucleotide sequence of SEQ ID NO: 38. In some embodiments, the polynucleotide encoding the KAR2 comprises the nucleotide sequence of SEQ ID NO: 38.

In some embodiments, the SEC1 comprises an amino acid sequence at least 70% (e.g., at least 70%, at least 75%, at least 80%, at least 85%, at least 90%, at least 99%, or at least 99%) identical to the amino acid sequence of SEQ ID NO: 39. In some embodiments, the SEC1 comprises the amino acid sequence of SEQ ID NO: 39. In some embodiments, the polynucleotide encoding the SEC1 comprises a nucleotide sequence at least 70% (e.g., at least 70%, at least 75%, at least 80%, at least 85%, at least 90%, at least 99%, or at least 99%) identical to the nucleotide sequence of SEQ ID NO: 40. In some embodiments, the polynucleotide encoding the SEC1 comprises the nucleotide sequence of SEQ ID NO: 40.

In some embodiments, the SLY1 comprises an amino acid sequence at least 70% (e.g., at least 70%, at least 75%, at least 80%, at least 85%, at least 90%, at least 99%, or at least 99%) identical to the amino acid sequence of SEQ ID NO: 41. In some embodiments, the SLY1 comprises the amino acid sequence of SEQ ID NO: 41. In some embodiments, the polynucleotide encoding the SLY1 comprises a nucleotide sequence at least 70% (e.g., at least 70%, at least 75%, at least 80%, at least 85%, at least 90%, at least 99%, or at least 99%) identical to the nucleotide sequence of SEQ ID NO: 42. In some embodiments, the polynucleotide encoding the SLY1 comprises the nucleotide sequence of SEQ ID NO: 42. In some embodiments, the GPX1 comprises an amino acid sequence at least 70% (e.g., at least 70%, at least 75%, at least 80%, at least 85%, at least 90%, at least 99%, or at least 99%) identical to the amino acid sequence of SEQ ID NO: 43. In some embodiments, the GPX1 comprises the amino acid sequence of SEQ ID NO: 43. In some embodiments, the polynucleotide encoding the GPX1 comprises a nucleotide sequence at least 70% (e.g., at least 70%, at least 75%, at least 80%, at least 85%, at least 90%, at least 99%, or at least 99%) identical to the nucleotide sequence of SEQ ID NO: 44. In some embodiments, the polynucleotide encoding the GPX1 comprises the nucleotide sequence of SEQ ID NO: 44.

In some embodiments, the recombinant yeast cell further comprises a polynucleotide encoding PDI (e.g., a PDI comprising the amino acid sequence of SEQ ID NO: 21). In some embodiments, the recombinant yeast cell further comprises a polynucleotide comprising the nucleotide sequence of SEQ ID NO: 22.

In some embodiments, the recombinant yeast cell further comprises a polynucleotide encoding PDI1 (e.g., a PDI1 comprising the amino acid sequence of SEQ ID NO: 23). In some embodiments, the recombinant yeast cell further comprises a polynucleotide comprising the nucleotide sequence of SEQ ID NO: 24.

In some embodiments, the recombinant yeast cell further comprises a polynucleotide encoding HAC1 (e.g., a HAC1 comprising the amino acid sequence of SEQ ID NO: 25). In some embodiments, the recombinant yeast cell further comprises a polynucleotide comprising the nucleotide sequence of SEQ ID NO: 26.

In some embodiments, the recombinant yeast cell further comprises a polynucleotide encoding HAC1 (e.g., a HAC1 comprising the amino acid sequence of SEQ ID NO: 25), a polynucleotide encoding ERO1 (e.g., an ERO1 comprising the amino acid sequence of SEQ ID NO: 35), and a polynucleotide encoding ERV2 (e.g., an ERV2 comprising the amino acid sequence of SEQ ID No: 33). In some embodiments, the recombinant yeast cell further comprises a polynucleotide comprising the nucleotide sequence of SEQ ID NO: 26, a polynucleotide comprising the nucleotide sequence of SEQ ID NO: 36, and a polynucleotide comprising the nucleotide sequence of SEQ ID NO: 34.

In some embodiments, the recombinant yeast cell further comprises a polynucleotide encoding PDI1 (e.g., a PDI comprising the amino acid sequence of SEQ ID NO: 23), a polynucleotide encoding ERV1 (e.g., an ERO1 comprising the amino acid sequence of SEQ ID NO: 31), and a polynucleotide encoding ERO2 (e.g., an ERV2 comprising the amino acid sequence of SEQ ID No: 27). In some embodiments, the recombinant yeast cell further comprises a polynucleotide comprising the nucleotide sequence of SEQ ID NO: 24, a polynucleotide comprising the nucleotide sequence of SEQ ID NO: 32, and a polynucleotide comprising the nucleotide sequence of SEQ ID NO: 28.

In some embodiments, each of the one or more polynucleotides encoding the one or more chaperones is operably linked to a promoter (e.g., a promoter selected from AOX1 promoter, GAP1 promoter, and CAT1 promoter).

In some embodiments, the one or more polynucleotides encoding the one or more chaperones are provided on one or more vectors (e.g., plasmids). In some embodiments, the one or more (e.g., 1, 2, 3, 4, 5 or more) polynucleotides encoding the one or more chaperones are provided on the same vector as the polynucleotide encoding the polypeptide comprising the tandem brazzein repeats. In some embodiments, the one or more (e.g., 1, 2, 3, 4, 5 or more) polynucleotides encoding the one or more chaperones are provided on different vectors as the polynucleotide encoding the polypeptide comprising the tandem brazzein repeats.

In some embodiments, the one or more polynucleotides encoding the one or more chaperones are integrated into the genome of the recombinant yeast cell. In some embodiments, the one or more polynucleotides are integrated into a HIS4 locus of the genome of the recombinant yeast cell.

In some embodiments, the method described herein comprising co-expressing the polypeptide comprising the tandem repeats of brazzein, and optionally the one or more chaperones, with one or more proteases to facilitate processing of the polypeptide comprising the tandem brazzein repeats, and/or release and secretion of the single brazzein repeat.

In some embodiments, the protease is selected from KEX1, KEX2, and Ste13.

Proteases of the same yeast strain as the yeast recombinant cell used for expression of the polypeptide may be used. Heterologous proteases from other yeast strains may also be used. As such, in some embodiments, the yeast recombinant cell used in the methods described herein further comprises one or more polynucleotides encoding one or more proteases selected from KEX1, KEX2, and Ste13.

In some embodiments, the one or more polynucleotides encoding the one or more proteases are provided on one or more vectors (e.g., plasmids). In some embodiments, the one or more (e.g., 1, 2, 3, or more) polynucleotides encoding the one or more proteases are provided on the same vector as the polynucleotide encoding the polypeptide comprising the tandem brazzein repeats. In some embodiments, the one or more (e.g., 1, 2, 3, 4, 5 or more) polynucleotides encoding the one or more proteases are provided on different vectors as the polynucleotide encoding the polypeptide comprising the tandem brazzein repeats.

In some embodiments, the one or more polynucleotides encoding the one or more proteases are integrated into the genome of the recombinant yeast cell. In some embodiments, the one or more polynucleotides are integrated into a HIS4 locus of the genome of the recombinant yeast cell.

Any yeast strain may be suitable as the recombinant yeast cell used in the methods described herein. Non-limiting examples of yeast strains include: Pichia pastoris, Pichia farinose, Pichia anomala, Pichia heedii, Pichia guilliermondii, Pichia kluyveri, Pichia membranifaciens, Pichia norvegensis, Pichia ohmeri, Pichia methanolica, Pichia subpelliculosa, Komagataella phaffii, Komagataella pastoris, Komagataella pseudopastoris, Candida vulgaris, Saccharomyces arboricolus, Saccharomyces bayanus, Saccharomyces bulderi, Saccharomyces cariocanus, Saccharomyces cariocus, Saccharomyces cerevisiae, Saccharomyces cerevisiae var. boulardii, Saccharomyces chevalieri, Saccharomyces dairenensis, Saccharomyces ellipsoideus, Saccharomyces eubayanus, Saccharomyces exiguous, Saccharomyces florentinus, Saccharomyces fragilis, Saccharomyces kudriavzevii, Saccharomyces martiniae, Saccharomyces mikatae, Saccharomyces monacensis, Saccharomyces norbensis, Saccharomyces paradoxus, Saccharomyces pastorianus, Saccharomyces spencerorum, Saccharomyces turicensis, Saccharomyces unisporus, Saccharomyces uvarum, and Saccharomyces zonatus. In some embodiments, the recombinant yeast cell in the methods described herein is a recombinant Pichia pastoris cell.

In some embodiments, the methods described herein further comprising isolating the brazzein.

### Synthetic Biology

Standard recombinant DNA and molecular cloning techniques used here are well known in the art and are described, for example, by Sambrook, J., Fritsch, E. F. and Maniatis, T. MOLECULAR CLONING: A LABORATORY MANUAL, 2nd ed.; Cold Spring Harbor Laboratory: Cold Spring Harbor, N.Y., 1989 (hereinafter “Maniatis”); and by Silhavy, T. J., Bennan, M. L. and Enquist, L. W. EXPERIMENTS WITH GENE FUSIONS; Cold Spring Harbor Laboratory: Cold Spring Harbor, N.Y., 1984; and by Ausubel, F. M. et al., IN CURRENT PROTOCOLS IN

MOLECULAR BIOLOGY, published by GREENE PUBLISHING AND WILEY-INTERSCIENCE, 1987; (the entirety of each of which is hereby incorporated herein by reference).

Unless defined otherwise, all technical and scientific terms used herein have the same meaning as commonly understood by one of ordinary skill in the art to which the disclosure belongs. Although any methods and materials similar to or equivalent to those described herein can be used in the practice or testing of the present disclosure, the preferred materials and methods are described below.

The disclosure will be more fully understood upon consideration of the following non-limiting Examples. It should be understood that these Examples, while indicating preferred embodiments of the subject technology, are given by way of illustration only. From the above discussion and these Examples, one skilled in the art can ascertain the essential characteristics of the subject technology, and without departing from the spirit and scope thereof, can make various changes and modifications of the subject technology to adapt it to various uses and conditions.

### Yeast Production Systems

Expression of proteins in prokaryotes is most often carried out in a yeast host cell with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: (1) to increase expression of recombinant protein; (2) to increase the solubility of the recombinant protein; and (3) to aid in the purification of the recombinant protein by acting as a ligand in affinity purification. Often, a proteolytic cleavage site is introduced at the junction of the fusion moiety and the recombinant protein to enable separation of the recombinant protein from the fusion moiety subsequent to purification of the fusion protein. Such vectors are within the scope of the present disclosure.

In an embodiment, the expression vector includes those genetic elements for expression of the recombinant polypeptide in yeast cells. The elements for transcription and translation in the yeast cell can include a promoter, a coding region for the protein complex, and a transcriptional terminator. We only used one standard E. coli expression system for this proof-of-concept work. The further modification and optimization is in progress.

A person of ordinary skill in the art will be aware of the molecular biology techniques available for the preparation of expression vectors. The polynucleotide used for incorporation into the expression vector of the subject technology, as described above, can be prepared by routine techniques such as polymerase chain reaction (PCR).

A number of molecular biology techniques have been developed to operably link DNA to vectors via complementary cohesive termini. In one embodiment, complementary homopolymer tracts can be added to the nucleic acid molecule to be inserted into the vector DNA. The vector and nucleic acid molecule are then joined by hydrogen bonding between the complementary homopolymeric tails to form recombinant DNA molecules.

In an alternative embodiment, synthetic linkers containing one or more restriction sites provide are used to operably link the polynucleotide of the subject technology to the expression vector. In an embodiment, the polynucleotide is generated by restriction endonuclease digestion. In an embodiment, the nucleic acid molecule is treated with bacteriophage T4 DNA polymerase or E. coli DNA polymerase I, enzymes that remove protruding, 3′-single-stranded termini with their 3′-5′-exonucleolytic activities and fill-in recessed 3′-ends with their polymerizing activities, thereby generating blunt-ended DNA segments. The blunt-ended segments are then incubated with a large molar excess of linker molecules in the presence of an enzyme that is able to catalyze the ligation of blunt-ended DNA molecules, such as bacteriophage T4 DNA ligase. Thus, the product of the reaction is a polynucleotide carrying polymeric linker sequences at its ends. These polynucleotides are then cleaved with the appropriate restriction enzyme and ligated to an expression vector that has been cleaved with an enzyme that produces termini compatible with those of the polynucleotide.

Alternatively, a vector having ligation-independent cloning (LIC) sites can be employed. The required PCR amplified polynucleotide can then be cloned into the LIC vector without restriction digest or ligation (Aslanidis and de Jong, NUCL. ACID. RES. 18 6069-74, (1990), Haun, et al, BIOTECHNIQUES 13, 515-18 (1992), which is incorporated herein by reference to the extent it is consistent herewith).

In an embodiment, in order to isolate and/or modify the polynucleotide of interest for insertion into the chosen plasmid, it is suitable to use PCR. Appropriate primers for use in PCR preparation of the sequence can be designed to isolate the required coding region of the nucleic acid molecule, add restriction endonuclease or LIC sites, place the coding region in the desired reading frame.

In an embodiment, a polynucleotide for incorporation into an expression vector of the subject technology is prepared by the use of PCR using appropriate oligonucleotide primers. The coding region is amplified, whilst the primers themselves become incorporated into the amplified sequence product. In an embodiment, the amplification primers contain restriction endonuclease recognition sites, which allow the amplified sequence product to be cloned into an appropriate vector.

The expression vectors can be introduced into host cells by conventional transformation or transfection techniques. Transformation of appropriate cells with an expression vector of the subject technology is accomplished by methods known in the art and typically depends on both the type of vector and cell. Suitable techniques include calcium phosphate or calcium chloride co-precipitation, DEAE-dextran mediated transfection, lipofection, chemoporation or electroporation.

Successfully transformed cells, that is, those cells containing the expression vector, can be identified by techniques well known in the art. For example, cells transfected with an expression vector of the subject technology can be cultured to produce polypeptides described herein. Cells can be examined for the presence of the expression vector DNA by techniques well known in the art.

The host cells can contain a single copy of the expression vector described previously, or alternatively, multiple copies of the expression vector.

Typically, the vector or cassette contains sequences directing transcription and translation of the relevant polynucleotide, a selectable marker, and sequences allowing autonomous replication or chromosomal integration. Suitable vectors comprise a region 5′ of the polynucleotide which harbors transcriptional initiation controls and a region 3′ of the DNA fragment which controls transcriptional termination. It is preferred for both control regions to be derived from genes homologous to the transformed host cell, although it is to be understood that such control regions need not be derived from the genes native to the specific species chosen as a host.

Initiation control regions or promoters, which are useful to drive expression of the recombinant polypeptide in the desired microbial host cell are numerous and familiar to those skilled in the art. Virtually any promoter capable of driving these genes is suitable for the subject technology including but not limited to CYCI, HIS3, GALI, GALIO, ADHI, PGK, PH05, GAPDH, ADCI, TRPI, URA3, LEU2, ENO, TPI (useful for expression in Saccharomyces); AOX1 (useful for expression in Pichia).

Termination control regions may also be derived from various genes native to the microbial hosts. A termination site optionally may be included for the microbial hosts described herein.

### Analysis of Sequence Similarity Using Identity Scoring

As used herein “sequence identity” refers to the extent to which two optimally aligned polynucleotide or peptide sequences are invariant throughout a window of alignment of components, e.g., nucleotides or amino acids. An “identity fraction” for aligned segments of a test sequence and a reference sequence is the number of identical components which are shared by the two aligned sequences divided by the total number of components in reference sequence segment, i.e., the entire reference sequence or a smaller defined part of the reference sequence.

As used herein, the term “percent sequence identity” or “percent identity” refers to the percentage of identical nucleotides in a linear polynucleotide sequence of a reference (“query”) polynucleotide molecule (or its complementary strand) as compared to a test (“subject”) polynucleotide molecule (or its complementary strand) when the two sequences are optimally aligned (with appropriate nucleotide insertions, deletions, or gaps totaling less than 20 percent of the reference sequence over the window of comparison). Optimal alignment of sequences for aligning a comparison window are well known to those skilled in the art and may be conducted by tools such as the local homology algorithm of Smith and Waterman, the homology alignment algorithm of Needleman and Wunsch, the search for similarity method of

Pearson and Lipman, and preferably by computerized implementations of these algorithms such as GAP, BESTFIT, FASTA, and TFASTA available as part of the GCG® Wisconsin Package® (Accelrys Inc., Burlington, MA). An “identity fraction” for aligned segments of a test sequence and a reference sequence is the number of identical components which are shared by the two aligned sequences divided by the total number of components in the reference sequence segment, i.e., the entire reference sequence or a smaller defined part of the reference sequence. Percent sequence identity is represented as the identity fraction multiplied by 100. The comparison of one or more polynucleotide sequences may be to a full-length polynucleotide sequence or a portion thereof, or to a longer polynucleotide sequence. For purposes of this disclosure “percent identity” may also be determined using BLASTX version 2.0 for translated nucleotide sequences and BLASTN version 2.0 for polynucleotide sequences.

The percent of sequence identity is preferably determined using the “Best Fit” or “Gap” program of the Sequence Analysis Software Package™ (Version 10; Genetics Computer Group, Inc., Madison, WI). “Gap” utilizes the algorithm of Needleman and Wunsch

(Needleman and Wunsch, JOURNAL OF MOLECULAR BIOLOGY 48:443-453, 1970) to find the alignment of two sequences that maximizes the number of matches and minimizes the number of gaps. “BestFit” performs an optimal alignment of the best segment of similarity between two sequences and inserts gaps to maximize the number of matches using the local homology algorithm of Smith and Waterman (Smith and Waterman, ADVANCES IN APPLIED MATHEMATICS, 2:482-489, 1981, Smith et al., NUCLEIC ACIDS RESEARCH 11:2205-2220, 1983). The percent identity is most preferably determined using the “Best Fit” program.

Useful methods for determining sequence identity are also disclosed in the Basic Local Alignment Search Tool (BLAST) programs which are publicly available from National Center Biotechnology Information (NCBI) at the National Library of Medicine, National Institute of

Health, Bethesda, Md. 20894; see BLAST Manual, Altschul et al., NCBI, NLM, NIH; Altschul et al., J. MOL. BIOL. 215:403-410 (1990); version 2.0 or higher of BLAST programs allows the introduction of gaps (deletions and insertions) into alignments; for peptide sequence BLASTX can be used to determine sequence identity; and, for polynucleotide sequence BLASTN can be used to determine sequence identity.

As used herein, the term “substantial percent sequence identity” refers to a percent sequence identity of at least about 70% sequence identity, at least about 80% sequence identity, at least about 85% identity, at least about 90% sequence identity, or even greater sequence identity, such as about 98% or about 99% sequence identity. Thus, one embodiment of the disclosure is a polynucleotide molecule that has at least about 70% sequence identity, at least about 80% sequence identity, at least about 85% identity, at least about 90% sequence identity, or even greater sequence identity, such as about 98% or about 99% sequence identity with a polynucleotide sequence described herein.

Identity is the fraction of amino acids that are the same between a pair of sequences after an alignment of the sequences (which can be done using only sequence information or structural information or some other information, but usually it is based on sequence information alone), and similarity is the score assigned based on an alignment using some similarity matrix. The similarity index can be any one of the following BLOSUM62, PAM250, or GONNET, or any matrix used by one skilled in the art for the sequence alignment of proteins.

Identity is the degree of correspondence between two sub-sequences (no gaps between the sequences). An identity of 25% or higher implies similarity of function, while 18-25% implies similarity of structure or function. Keep in mind that two completely unrelated or random sequences (that are greater than 100 residues) can have higher than 20% identity. Similarity is the degree of resemblance between two sequences when they are compared. This is dependent on their identity.

As is evident from the foregoing description, certain aspects of the present disclosure are not limited by the particular details of the examples illustrated herein, and it is therefore contemplated that other modifications and applications, or equivalents thereof, will occur to those skilled in the art. It is accordingly intended that the claims shall cover all such modifications and applications that do not depart from the spirit and scope of the present disclosure.

Moreover, unless defined otherwise, all technical and scientific terms used herein have the same meaning as commonly understood by one of ordinary skill in the art to which the disclosure belongs. Although any methods and materials similar to or equivalent to or those described herein can be used in the practice or testing of the present disclosure, the preferred methods and materials are described above.

### Orally Consumable Products

Some aspects of the present disclosure provide compositions comprising the brazzein produced using the methods described herein. In some embodiments, the brazzein produced using the methods described herein can be used, e.g., as sweeteners, in products, e.g., consumable products (e.g., orally consumable products).

In some embodiments, the consumable products can be, for example, a food product, a beverage product, a nutraceutical, a pharmaceutical, a dietary supplement, a dental hygienic composition, an edible gel composition, a cosmetic product and a tabletop flavoring.

Any one of the consumable products (e.g., orally consumable products) can also have at least one additional sweetener. The at least one additional sweetener can be a natural high intensity sweetener, for example. The additional sweetener can be selected from a stevia extract, a steviol glycoside, stevioside, rebaudioside A, rebaudioside B, rebaudioside C, rebaudioside D, rebaudioside D2, rebaudioside E, rebaudioside F, rebaudioside M, rebaudioside V, rebaudioside W, rebaudioside Z1, rebaudioside Z2, rebaudioside D3, dulcoside A, rubusoside, rebaudioside N, rebaudioside I, rebaudioside G, rebaudioside WB1, rebaudioside WB2, rebaudioside R6-2A, rebaudioside R6-2B, rebaudioside R6-4A, rebaudioside R6-4B, rebaudioside R7-2, steviolbioside, sucrose, high fructose corn syrup, fructose, glucose, xylose, arabinose, rhamnose, erythritol, xylitol, mannitol, sorbitol, inositol, AceK, aspartame, neotame, sucralose, saccharine, naringin dihydrochalcone (NarDHC), neohesperidin dihydrochalcone (NDHC), rubusoside, mogroside IV, siamenoside I, mogroside V, monatin, thaumatin, monellin, L-alanine, glycine, Lo Han Guo, hernandulcin, phyllodulcin, trilobtain, and combinations thereof.

Any one of the consumable products (e.g., orally consumable products) can also have at least one additive. The additive can be, for example, a carbohydrate, a polyol, an amino acid or salt thereof, a polyamino acid or salt thereof, a sugar acid or salt thereof, a nucleotide, an organic acid, an inorganic acid, an organic salt, an organic acid salt, an organic base salt, an inorganic salt, a bitter compound, a flavorant, a flavoring ingredient, an astringent compound, a protein, a protein hydrolysate, a surfactant, an emulsifier, a flavonoids, an alcohol, a polymer, and combinations thereof.

In some embodiments, the present disclosure provides a beverage product comprising a sweetening amount of brazzein produced using the methods described herein. Any one of the beverage products can be, for example, a carbonated beverage product and a non-carbonated beverage product. Any one of the beverage products can also be, for example, a soft drink, a fountain beverage, a frozen beverage; a ready-to-drink beverage; a frozen and ready-to-drink beverage, coffee, tea, a dairy beverage, a powdered soft drink, a liquid concentrate, flavored water, enhanced water, fruit juice, a fruit juice flavored drink, a sport drink, and an energy drink.

In some embodiments, any one of the beverage products of the present disclosure can include one or more beverage ingredients such as, for example, acidulants, fruit juices and/or vegetable juices, pulp, etc., flavorings, coloring, preservatives, vitamins, minerals, electrolytes, erythritol, tagatose, glycerine, and carbon dioxide. Such beverage products may be provided in any suitable form, such as a beverage concentrate and a carbonated, ready-to-drink beverage.

In certain embodiments, any one of the beverage products of the present disclosure can have any of numerous different specific formulations or constitutions. The formulation of a beverage product of the present disclosure can vary to a certain extent, depending upon such factors as the product's intended market segment, its desired nutritional characteristics, flavor profile, and the like. For example, in certain embodiments, it can generally be an option to add further ingredients to the formulation of a particular beverage product. For example, additional (i.e., more and/or other) sweeteners can be added, flavorings, electrolytes, vitamins, fruit juices or other fruit products, tastents, masking agents and the like, flavor enhancers, and/or carbonation typically may be added to any such formulations to vary the taste, mouthfeel, nutritional characteristics, etc.

Exemplary flavorings can be, for example, cola flavoring, citrus flavoring, and spice flavorings. In some embodiments, carbonation in the form of carbon dioxide can be added for effervescence. In other embodiments, preservatives can be added, depending upon the other ingredients, production technique, desired shelf life, etc. In certain embodiments, caffeine can be added. In some embodiments, the beverage product can be a cola-flavored carbonated beverage, characteristically containing carbonated water, sweetener, kola nut extract and/or other flavoring, caramel coloring, one or more acids, and optionally other ingredients.

As used herein, “dietary supplement(s)” refers to compounds intended to supplement the diet and provide nutrients, such as vitamins, minerals, fiber, fatty acids, amino acids, etc. that may be missing or may not be consumed in sufficient quantities in a diet. Any suitable dietary supplement known in the art may be used. Examples of suitable dietary supplements can be, for example, nutrients, vitamins, minerals, fiber, fatty acids, herbs, botanicals, amino acids, and metabolites.

As used herein, “nutraceutical(s)” refers to compounds, which includes any food or part of a food that may provide medicinal or health benefits, including the prevention and/or treatment of disease or disorder (e.g., fatigue, insomnia, effects of aging, memory loss, mood disorders, cardiovascular disease and high levels of cholesterol in the blood, diabetes, osteoporosis, inflammation, autoimmune disorders, etc.). Any suitable nutraceutical known in the art may be used. In some embodiments, nutraceuticals can be used as supplements to food and beverages and as pharmaceutical formulations for enteral or parenteral applications which may be solid formulations, such as capsules or tablets, or liquid formulations, such as solutions or suspensions.

In some embodiments, dietary supplements and nutraceuticals can further contain protective hydrocolloids (such as gums, proteins, modified starches), binders, film-forming agents, encapsulating agents/materials, wall/shell materials, matrix compounds, coatings, emulsifiers, surface active agents, solubilizing agents (oils, fats, waxes, lecithins, etc.), adsorbents, carriers, fillers, co-compounds, dispersing agents, wetting agents, processing aids (solvents), flowing agents, taste-masking agents, weighting agents, jellyfying agents, gel-forming agents, antioxidants and antimicrobials.

As used herein, a “gel” refers to a colloidal system in which a network of particles spans the volume of a liquid medium. Although gels mainly are composed of liquids, and thus exhibit densities similar to liquids, gels have the structural coherence of solids due to the network of particles that spans the liquid medium. For this reason, gels generally appear to be solid, jelly-like materials. Gels can be used in a number of applications. For example, gels can be used in foods, paints, and adhesives. Gels that can be eaten are referred to as “edible gel compositions.” Edible gel compositions typically are eaten as snacks, as desserts, as a part of staple foods, or along with staple foods. Examples of suitable edible gel compositions can be, for example, gel desserts, puddings, jams, jellies, pastes, trifles, aspics, marshmallows, gummy candies, and the like. In some embodiments, edible gel mixes generally are powdered or granular solids to which a fluid may be added to form an edible gel composition. Examples of suitable fluids can be, for example, water, dairy fluids, dairy analogue fluids, juices, alcohol, alcoholic beverages, and combinations thereof. Examples of suitable dairy fluids can be, for example, milk, cultured milk, cream, fluid whey, and mixtures thereof. Examples of suitable dairy analogue fluids can be, for example, soy milk and non-dairy coffee whitener.

As used herein, the term “gelling ingredient” refers to any material that can form a colloidal system within a liquid medium. Examples of suitable gelling ingredients can be, for example, gelatin, alginate, carageenan, gum, pectin, konjac, agar, food acid, rennet, starch, starch derivatives, and combinations thereof. It is well known to those in the art that the amount of gelling ingredient used in an edible gel mix or an edible gel composition can vary considerably depending on a number of factors such as, for example, the particular gelling ingredient used, the particular fluid base used, and the desired properties of the gel. Gel mixes and gel compositions of the present disclosure can be prepared by any suitable method known in the art. In some embodiments, edible gel mixes and edible gel compositions of the present disclosure can be prepared using other ingredients in addition to the gelling agent. Examples of other suitable ingredients can be, for example, a food acid, a salt of a food acid, a buffering system, a bulking agent, a sequestrant, a cross-linking agent, one or more flavors, one or more colors, and combinations thereof.

Pharmaceutical compositions are also provided comprising brazzein produced using the methods described herein. In some embodiments, any one of the pharmaceutical compositions of the present disclosure can be used to formulate pharmaceutical drugs containing one or more active agents that exert a biological effect. Accordingly, in some embodiments, any one of the pharmaceutical compositions of the present disclosure can contain one or more active agents that exert a biological effect. Suitable active agents are well known in the art (e.g., The Physician's Desk Reference). Such compositions can be prepared according to procedures well known in the art, for example, as described in Remington's Pharmaceutical Sciences, Mack Publishing Co., Easton, Pa., USA.

The brazzein produced using the methods described herein can be used with any suitable dental and oral hygiene compositions known in the art. Examples of suitable dental and oral hygiene compositions can be, for example, toothpastes, tooth polishes, dental floss, mouthwashes, mouth rinses, dentrifices, mouth sprays, mouth refreshers, plaque rinses, dental pain relievers, and the like. Dental and oral hygiene compositions comprising any one of the rebaudiosides provided herein are also provided.

As used herein, “food product composition(s)” refers to any solid or liquid ingestible material that can, but need not, have a nutritional value and be intended for consumption by humans and animals.

Examples of suitable food product compositions can be, for example, confectionary compositions, such as candies, mints, fruit flavored drops, cocoa products, chocolates, and the like; condiments, such as ketchup, mustard, mayonnaise, and the like; chewing gums; cercal compositions; baked goods, such as breads, cakes, pies, cookies, and the like; dairy products, such as milk, cheese, cream, ice cream, sour cream, yogurt, sherbet, and the like; tabletop sweetener compositions; soups; stews; convenience foods; meats, such as ham, bacon, sausages, jerky, and the like; gelatins and gelatin-like products such as jams, jellies, preserves, and the like; fruits; vegetables; egg products; icings; syrups including molasses; snacks; nut meats and nut products; and animal feed.

Food product compositions can also be herbs, spices and seasonings, natural and synthetic flavors, and flavor enhancers, such as monosodium glutamate. In some embodiments, any one of the food product compositions can be, for example, prepared packaged products, such as dietetic sweeteners, liquid sweeteners, granulated flavor mixes, pet foods, livestock feed, tobacco, and materials for baking applications, such as powdered baking mixes for the preparation of breads, cookies, cakes, pancakes, donuts and the like. In other embodiments, any one of the food product compositions can also be diet and low-calorie food and beverages containing little or no sucrose.

### EXAMPLES

### Example 1-Expression of brazzein in Pichia pastoris

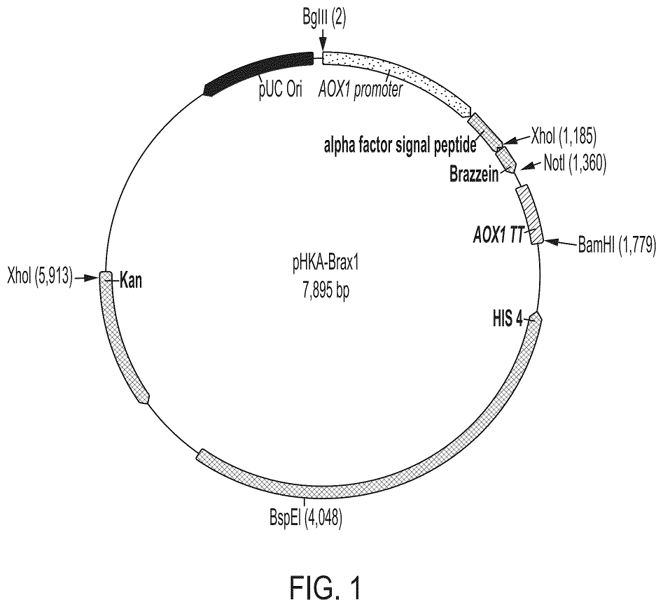

To demonstrate the transformation of Pichia pastoris cells to produce several engineered Pichia strains suitable for secreted brazzein production, the following experiments were conducted. Full-length DNA fragment of the brazzein gene (SEQ ID NO: 2) was codon optimized for Pichia pastoris expression and synthesized for use in the transformation of the Pichia pastoris cells. The brazzein fragment was inserted in frame after a nucleotide sequence encoding the a mating factor signal peptide in a pHKA vector (a modified Pichia expression vector that is a high copy number vector) in order to generate a single copy plasmid (pHKA-Brax1, FIG. 1). In the pHKA-Brax1 plasmid, each expression cassette contains the AOX1 promoter, an alpha (α) mating factor signal peptide-brazzein fusion gene and an AOX1 transcription terminator. The α mating factor signal peptide-brazzein fusion protein can be cleaved by endogenous Kex2 protease and release the mature brazzein peptide (SEQ ID NO: 1) into the extracellular space.

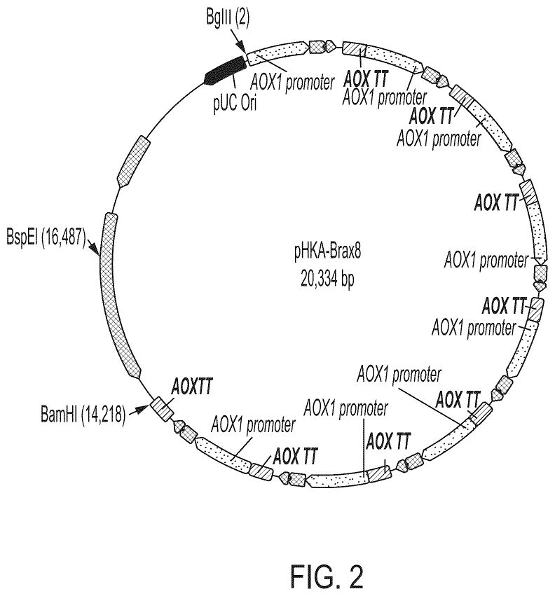

To generate the multiple copies of the expression cassette in vitro, the above plasmid was digested with BspEI and BglII or BspEI and BamHI. The fragments containing the brazzein coding sequence were gel-purified and then ligated together. Resulting E. coli colonies were screened by digestion with BglII and BamHI to find colonies with an insert that is double the size of the signal expression cassette, which are plasmids containing 2 expression cassettes. This procedure was repeated on the 2 copies plasmid, then on a 4 copies plasmid to generate a pHKA Pichia expression plasmid harboring 8 copies of identical brazzein expression cassettes (pHKA-Brax8, FIG. 2).

Identified plasmids were linearized by BspEI digestion. The linearized expression plasmid was transformed into Pichia pastoris (GS115) cells using known methods and the expression cassette was integrated into the His 4 locus of Pichia genome. After screening, the positive strains were identified, as summarized in Table 1.

TABLE 1
Summary of Pichia pastoris strains

| | | | Amino | DNA | | Integra- |
|---|---|---|---|---|---|---|
| Strain | Plasmid | Plasmid | acid | se- | Description | tion |
| name | name | map | sequence | quence | of plasmid | locus |
| Brax1 | pHKA- | FIG. 1 | SEQ ID | SEQ ID | 1 brazzein | His 4 |
| | Brax1 | | NO: 1 | NO: 2 | expression | |
| | | | | | cassette | |
| Brax8 | pHKA- | FIG. 2 | SEQ ID | SEQ ID | 8 brazzein | His 4 |
| | Brax8 | | NO: 1 | NO: 2 | expression | |
| | | | | | cassettes | |

To demonstrate brazzein production, the following experiment was conducted. A single colony of the Pichia pastoris strains were inoculated in BMGY medium in a 24-well plate or baffled flask and grown at 28-30° C. in a shaking incubator (250-300 rpm) until the culture reached an OD600 of 2-6 (log-phase growth). The cells were harvested by centrifuging and resuspended to an OD600 of 1.0 in BMM/BMMY medium to induce expression. 100% methanol was added to the BMMY medium to a final concentration of 1% methanol every 24 hours to maintain induction of expression. The medium was harvested at different induction time by centrifugation and subjected to SDS-PAGE, HPLC and LC-MS analysis as described below.

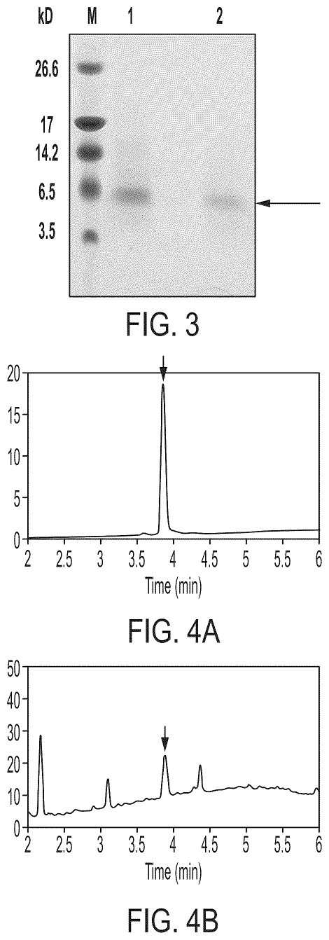

Brazzein (des-pyrE-bra) is around a 6.4 kD peptide. In order to identify brazzein production, multiple methods were used to detect brazzein in these samples. Medium samples were subjected to electrophoresis on a 10-20% Tricine SDS-PAGE gel. As shown in FIG. 3, there are 6.4 kD bands in both the Brax1 sample and the Brax8 sample, indicating brazzein production in engineered Pichia strain. Brax8 strain has higher brazzein production because there is darker band in the Brax8 sample compared to the Brax1 sample (FIG. 3). Increased brazzein expression cassette copies can increase brazzein production in Pichia. In order to confirm brazzein production, these samples were analyzed by HPLC and LC-MS analysis comparing to a brazzein standard.

HPLC analysis was performed using a Hypersil Gold C18 column. A linear gradient increased from 10%-50% 0.1% TFA in water: 0.1% TFA in acetonitrile over 10 minutes then dropped down to 10% for an additional 5 minutes at a flow rate of 0.5 mL/min. Folded brazzein standard clutes at 3.9 minutes. When the supernatant of Brax8 strain was precipitated with ammonium sulfate and resuspended in water, a peak with a same retention time to the standard was observed (FIG. 4).

Samples were analyzed by LC-MS using the Waters BEH C18 column. Mobile phase A was 0.1% formic acid in water, and mobile phase B was 0.1% formic acid in acetonitrile. The flow rate was 0.2 ml/minute. Mass spectrometry analysis of the samples was done on the Q Exactive Hybrid Quadrupole-Orbitrap Mass Spectrometer (Thermo Fisher Scientific) with an optimized method in positive ion mode. The produced peptide in the Brax8 strain has same retention time as brazzein (des-PyrE-bra). The produced peptide has the same mass ([M+H]+: 6357.74 m/z) as the brazzein standard (des-pyrE-bra, four disulfide bonds).

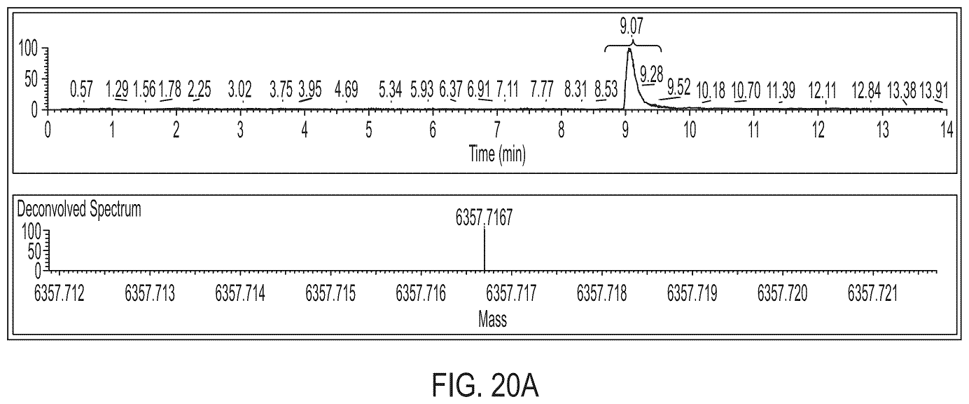

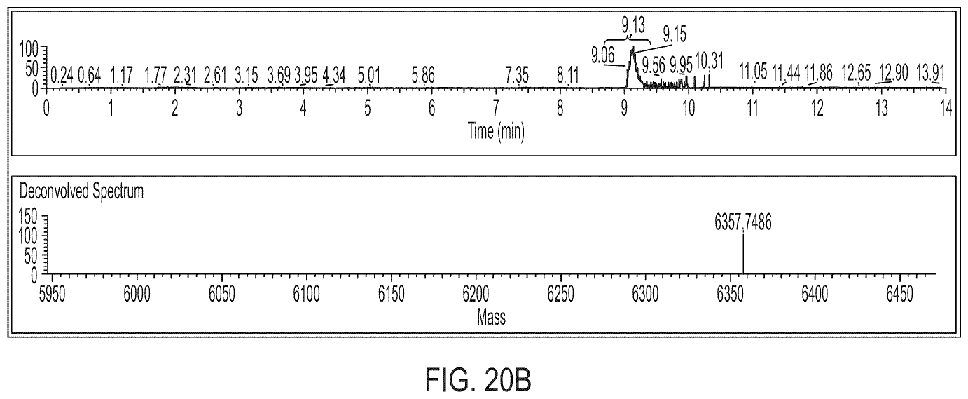

As shown in FIG. 19, 6.4 kD bands could be detected in all samples containing 1-5 EA repeat numbers. No cleavage was found for the KR linker without EA. In order to confirm brazzein production, these samples were analyzed by LC-MS analysis comparing to brazzein standard. The product brazzein was characterized by the same MS as brazzein standard (FIG. 20). The foregoing results provided the evidence supporting brazzein production in engineered Pichia strains according to the tandem repeat brazzein fusion protein strategy.

### Example 2-Expression of Brazzein Using Tandem Repeat Recombinant Brazzein Fusion Protein

In yeasts including P. pastoris, the alpha mating factor is expressed as a tandem repeat fragment. The mature α mating factor is spaced by amino acid sequences that follow the pattern KR (EA) x. The structure of the gene including the number of repeats of the α mating factor and the length of spacer varies by species. For Pichia, the gene has 10 repeats of the mature α mating factor with spacers that vary from 5 to 9 repeats of EA. Processing the α mating factor polypeptide involves the combined action of 3 proteases. KEX2 is an endopeptidase cleaves at the C-terminal side of KR. KEX1 is a carboxypeptidase that removes K and R residues from the C-terminus of peptides. Ste13 is an endopeptidase that cleaves after repeats of EA residues.

The amount of brazzein produced by P. pastoris can be increased by expressing the peptide sweetener in a tandem repeat that mimics the yeast α mating factor. In this study 3 different spacers were tested: Spacer 1 (KR; (SEQ ID NO: 3, encoded by SEQ ID NO: 4), spacer 2 (KREA; SEQ ID NO: 5, encoded by SEQ ID NO: 6), spacer 3 (KREAEAEAEAEA; SEQ ID NO: 7, encoded by SEQ ID NO: 8), spacer 4 (KREAEA; SEQ ID NO: 47, encoded by SEQ ID NO: 48), sequence 5 (KREAEAEA; SEQ ID NO: 49, encoded by SEQ ID NO: 50), and sequence 6 (KREAEAEAEA (SEQ ID NO: 51, encoded by SEQ ID NO: 52).

The 2 tandem repeat plasmids were generated by Gibson assembly. Forward and reverse primers were designed to add the spacer before and after the brazzein coding sequence respectively. The primers were paired with a forward and reverse primer annealing to the KAN marker of the pHKA Pichia expression vector. The pHKA-Brax 1 brazzein plasmid was used as template for PCR. The band of the correct size was gel purified, digested with DpnI to remove any template plasmid and assembled with 2× Gibson master mix. The resulting plasmids were sequenced to confirm insertion of the spacer and that no mutations to the fusion brazzein gene occurred. The same cloning strategy was used to insert different spacers in tandem repeat brazzein fusion protein. The same procedure was repeated to generate more tandem repeats plasmids. Eventually, multiple constructs were generated and transformed into Pichia cells for screening (Table 2).

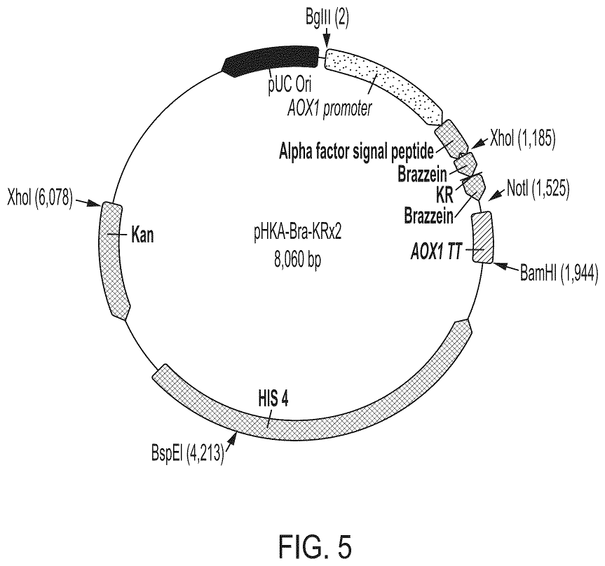

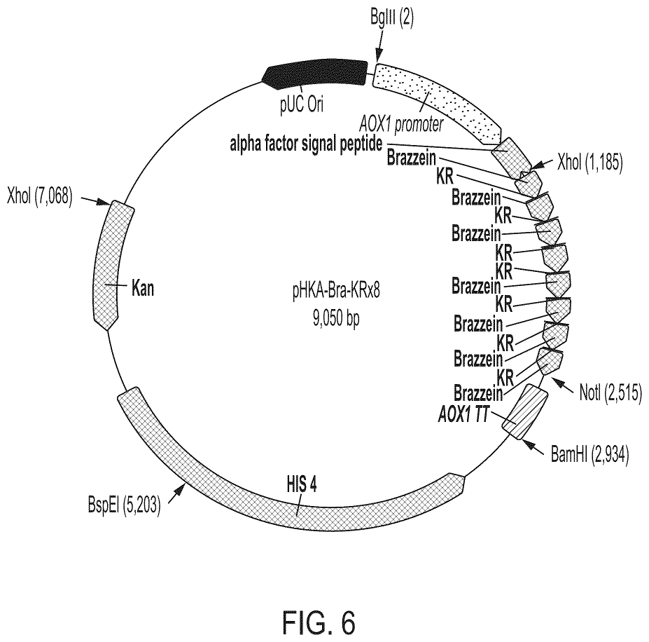

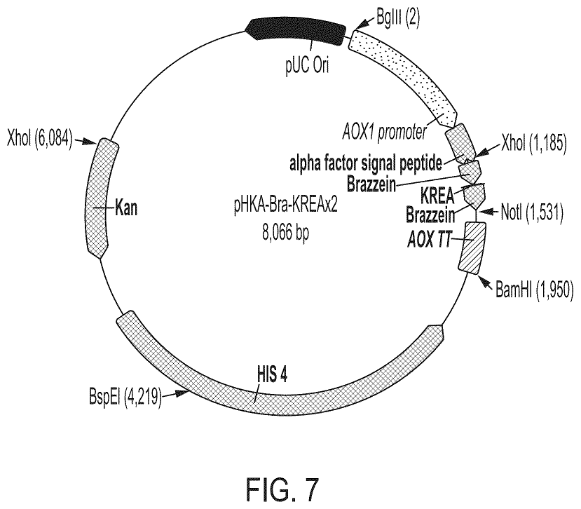

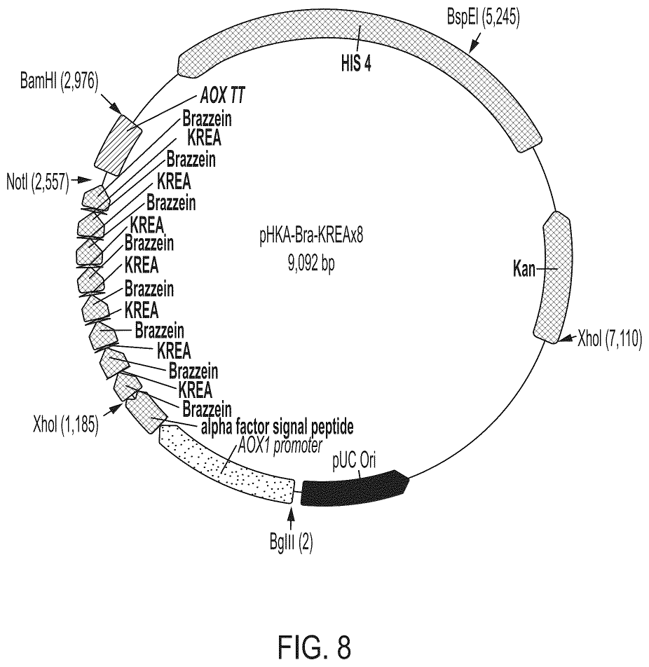

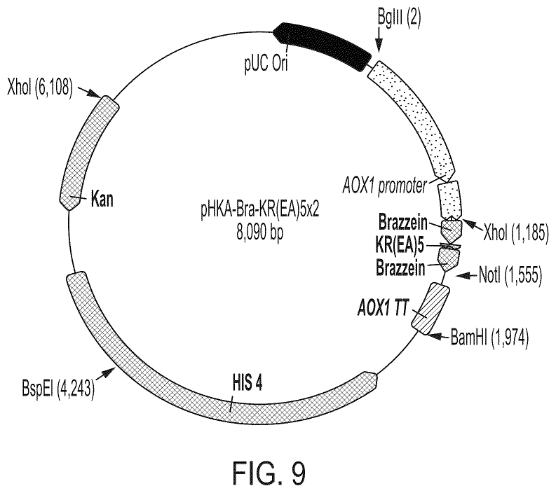

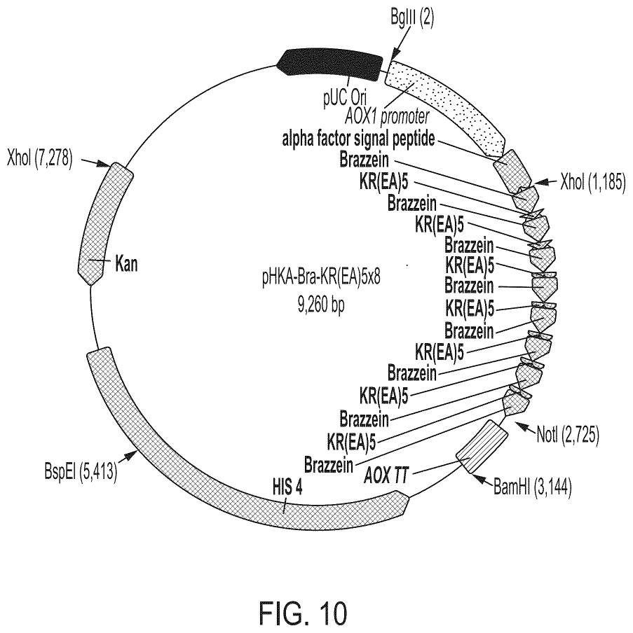

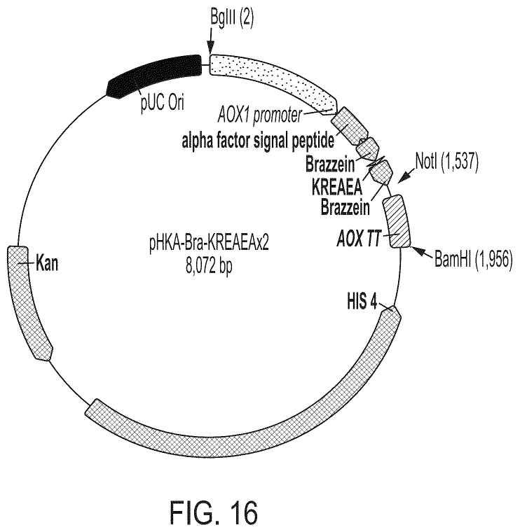

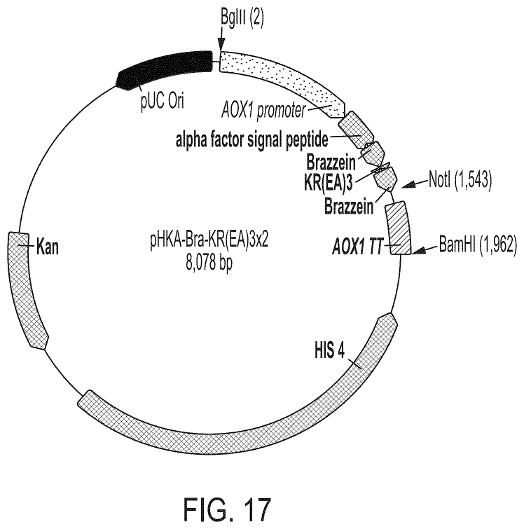

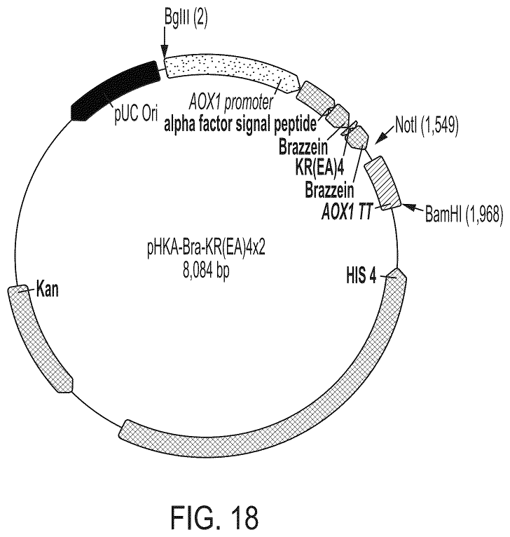

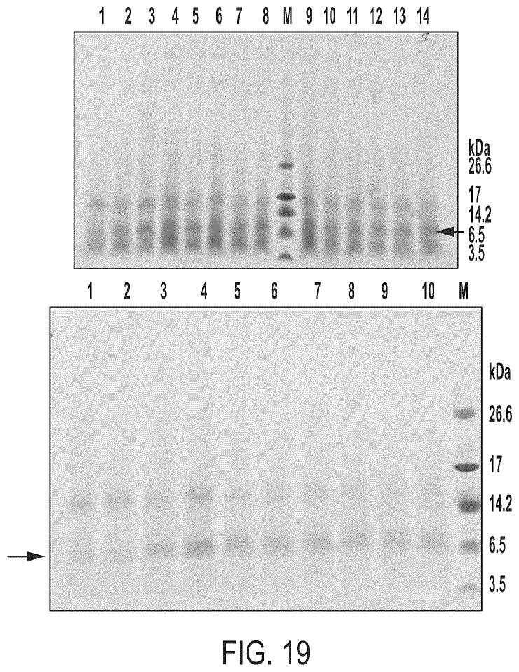

TABLE 2
Summary of strains and plasmids

| | | Fusion | Fusion | | |
|---|---|---|---|---|---|
| | | protein | gene | | |
| | | Amino acid | DNA | Plasmid | Integration |
| Strain name | Plasmid name | sequence | sequence | map | locus |
| Bra-KRx2 | pHKA-Bra-KRx2 | SEQ ID | SEQ ID | FIG. 5 | His 4 |
| | | NO: 9 | NO: 10 | | |
| Bra-KRx8 | pHKA-Bra-KRx8 | SEQ ID | SEQ ID | FIG. 6 | His 4 |
| | | NO: 11 | NO: 12 | | |
| Bra-KREAx2 | pHKA-Bra- | SEQ ID | SEQ ID | FIG. 7 | His 4 |
| | KREAx2 | NO: 13 | NO: 14 | | |
| Bra-KREAx8 | pHKA-Bra- | SEQ ID | SEQ ID | FIG. 8 | His 4 |
| | KREAx8 | NO: 15 | NO: 16 | | |
| Bra-KR(EA)5x2 | pHKA-Bra- | SEQ ID | SEQ ID | FIG. 9 | His 4 |
| | KR(EA)5x2 | NO: 17 | NO: 18 | | |
| Bra-KR(EA)5X8 | pHKA-Bra- | SEQ ID | SEQ ID | FIG. 10 | His 4 |
| | KR(EA)5X8 | NO: 19 | NO: 20 | | |
| Bra-KREAEAx2 | pHKA-Bra- | SEQ ID | SEQ ID | FIG. 16 | His 4 |
| | KREAEAx2 | NO: 53 | NO: 54 | | |
| Bra-KR(EA)3x2 | pHKA-Bra- | SEQ ID | SEQ ID | FIG. 17 | His 4 |
| | KR(EA)3x2 | NO: 55 | NO: 56 | | |
| Bra-KR(EA)4x2 | pHKA-Bra- | SEQ ID | SEQ ID | FIG. 18 | His 4 |
| | KR(EA)4x2 | NO: 57 | NO: 58 | | |

To demonstrate brazzein production, the following experiment was conducted. A single colony of the Pichia pastoris strains were inoculated in BMGY medium in a 24-well plate or baffled flask and grown at 28-30° C. in a shaking incubator (250-300 rpm) until the culture reached an OD600 of 2-6 (log-phase growth). The cells were harvested by centrifuging and resuspended to an OD600 of 1.0 in BMM/BMMY medium to induce expression. 100% methanol was added to the BMMY medium to a final concentration of 1% methanol every 24 hours to maintain induction of expression. The medium was harvested at different induction time by centrifugation and subjected to SDS-PAGE, HPLC and LC-MS analysis as described below.

### Example 3-Improvement Brazzein Folding, Secretion and Production in Pichia pastoris

For the brazzein peptide to have its characteristic sweetness, the four disulfide bonds must be formed. While P. pastoris is a good host for disulfide bond formation, the overexpression of a heterologous disulfide bonded product can overwhelm the cell's native capacity. To improve the amount of correctly folded brazzein produced by above identified Pichia strains, a series of chaperone and proteins related to protein expression, secretion, folding and disulfide formation were overexpressed in brazzein producing Pichia strains. These chaperones and proteins were selected from P. pastoris or plant to be heterologous expressed with brazzein in Pichia. While none of the chaperones involved in disulfide bond formation in P. brazzeana have been identified, the disulfide bond formation system of the closely related Arabidopsis thaliana has been characterized (Table 3).

Protein disulfide isomerase (PDI) is a chaperone localized primarily in the endoplasmic reticulum that aids in forming disulfide bonds between cysteine residues. Overexpressing PDI in P. pastoris has also been shown to improve the expression of certain non-disulfide bond containing proteins. ER oxidoreductin (ERO) proteins work in tandem with PDI by donating oxidating equivalents for disulfide bond formation. ERVs are a family of sulfhydryl oxidases that play a similar role as EROs but may also directly catalyze disulfide bond formation. HAC1 is a transcriptional regulator of the unfolded protein response in P. pastoris. GPX1 is a cytosolic peroxidase that is involved in cellular redox balancing. KAR2 codes for the ER chaperone BiP that aids in proper folding and directs misfolded proteins to be degraded. The genes SLY1 and SEC1 regulate vesicle traffic from the ER to the Golgi and from the Golgi to the extracellular membrane respectively. Overexpression of KEX1, KEX2, and Ste13 enzymes from P. pastoris or the heterologous expression of these proteases from other yeast species can improve the release of brazzein expressed as a tandem repeat. All selected transcription regulator, chaperones and disulfide bond formation related proteins were list in Table 3.

TABLE 3
Selection of candidates for co-expression
in brazzein production strain.

| | GenBank accession | Amino acid | DNA |
|---|---|---|---|
| name | number | sequence | sequence |
| PpPDI | CAC33587.1 | SEQ ID NO: 21. | SEQ ID NO: 22 |
| AtPDI1 | Q8VX13.1 | SEQ ID NO: 23 | SEQ ID NO: 24 |
| HAC1 | 254565857 | SEQ ID NO: 25 | SEQ ID NO: 26 |
| AtERO1 | NP_177372.1 | SEQ ID NO: 27 | SEQ ID NO: 28 |
| AtERO2 | NP_181430.2 | SEQ ID NO: 29 | SEQ ID NO: 30 |
| AtERV1 | XM_002492510.1 | SEQ ID NO: 31 | SEQ ID NO: 32 |
| PpERV2 | ANZ73509.1 | SEQ ID NO: 33 | SEQ ID NO: 34 |
| PpERO1 | ANZ74048.1 | SEQ ID NO: 35 | SEQ ID NO: 36 |
| PpKAR2 | ANZ77450.1 | SEQ ID NO: 37 | SEQ ID NO: 38 |
| PpSEC1 | CAY67514.1 | SEQ ID NO: 39 | SEQ ID NO: 40 |
| PpSLY1 | CCA40908.1 | SEQ ID NO: 41 | SEQ ID NO: 42 |
| PpGPX1 | BAH57503.1 | SEQ ID NO: 43 | SEQ ID NO: 44 |

Chaperone genes were cloned into a modified pPICZ-alpha vector that has an NdeI site after the AOX1 promoter in place of the EcoRI site. To construct vectors with multiple chaperones or multiple copies of the same chaperone, the vector containing chaperone to be added was digested with BglII and BamHI to release the expression cassette. The expression cassette was then ligated into a second chaperone vector linearized with BamHI. A BglII and BamHI digest was performed on the resulting plasmids to confirm the insertion of the chaperone in the proper orientation. Generated vectors were linearized at the AOX1 promoter with SacI restriction enzyme and used to transform GS115 P. pastoris with brazzein expression cassettes integrated at the HIS4 locus (Brax8). Colonies with chaperone integration were selected on YPD-Zeocin and confirmed by colony PCR.

As the chaperones selected often work in tandem with other enzymes to improve secretion or disulfide bonding, expression vectors with multiple genes were generated. To ensure integration at the AOX1 locus, the promoter from the added genes needed to be replaced. The GAP1 and CAT1 promoters were amplified from GS115 genomic DNA with primers that added BglII and NdeI sites to the 5′ and 3′ ends respectively. The AOX1 promoter was then excised from the pPICZ vectors with a BglII/NdeI digest and replaced with the GAP1 and CAT1 promoters. Various combination of different expression cassettes was generated as described as Example 1 using BglII, BamHI and BspEI digestion. All plasmids were listed in Table 4.

TABLE 4
Summary of selected plasmids of single and combination
of different chaperones for co-expression

| | | | Parent |
|---|---|---|---|
| Name | Gene | Promoter | strain |
| pPICZ-PpPDI | PpPDI (SEQ ID | AOX1 | Brax8 |
| | NO: 22) | | |
| pPICZ-AtPDI | AtPDI (SEQ ID | AOX1 | Brax8 |
| | NO: 24) | | |
| pPICZ-HAC1 | HAC1 (SEQ ID | AOX1 | Brax8 |
| | NO: 26) | | |
| pPICZ-PpPDI/PpERO1/PpERV2 | PpPDI (SEQ ID | AOX1 | Brax8 |
| | NO: 22) | | |
| | PpERO1 (SEQ ID | GAP1 | |
| | NO: 36) | | |
| | PpERV2 (SEQ ID | CAT1 | |
| | NO: 34) | | |
| pPICZ-AtPDI/AtERV1/AtERO1 | AtPDI (SEQ ID | AOX1 | Brax8 |
| | NO: 24) | | |
| | AtERV1(SEQ ID | CAT1 | |
| | NO: 32) | | |
| | AtERO1 (SEQ ID | CAT1 | |
| | NO: 28) | | |

To demonstrate improvement of brazzein production with chaperone co-expression, confirmed colonies were grown overnight in BMGY media in 24-well plates. The next day the cells were resuspended in 2 mL BMMY media to an OD600 of 1.0. The cultures were induced at 30° C. for 48 hours with the additional feeding of 1% methanol to each well twice daily. The cells were harvested and spun down. The improved secretion of folded brazzein was measured on HPLC after precipitation from the supernatant with ammonium sulfate. 250 μL supernatant was added to 1 mL saturated ammonium sulfate solution and incubated at 4° C. with gentle rocking for 1 hour. The plate was then spun at 5000×g for 20 minutes at 4° C. The plate was then inverted to remove the supernatant and patted dry on clean paper towels. The pellets were resuspended in 100 μL water and run on a C18 HPLC column to measure the amount of brazzein. Samples that showed improved production of brazzein were run on LC-MS to confirm the presence of all 4 disulfide bonds.

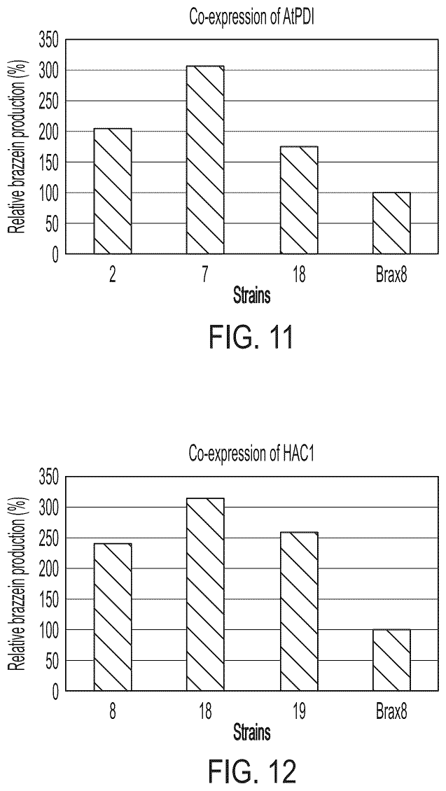

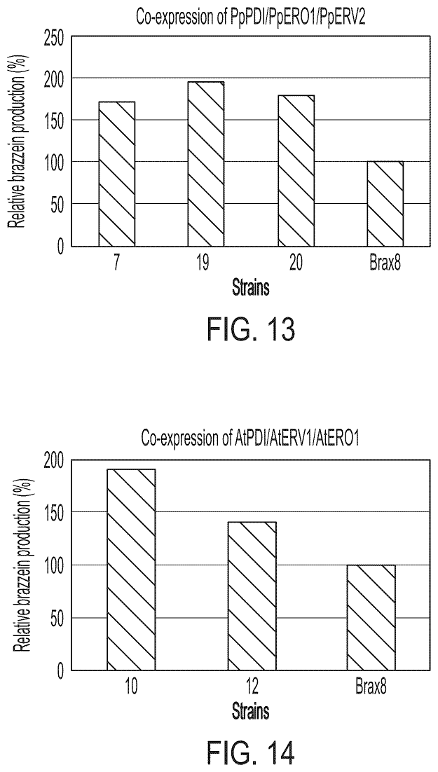

Co-expression of AtPD1 can increase brazzein production. As shown in FIG. 11, three co-expression strains (2, 7 and 18) have higher brazzein production than the parent strain (Brax8). Co-expression HAC1 transcription regulator also can increase brazzein production (FIG. 12). Co-expression of multiple chaperones and disulfide bond formation related proteins (PD1, ERO and ERV), either from Pichia pastoris and Arabidopsis, can increase brazzein production in Brax8 strain (FIG. 13 and FIG. 14) and improve correct folded brazzein production.

### Example 4-Fermentation, Purification and Identification or Brazzein

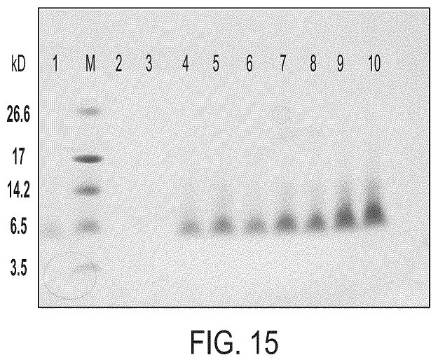

Identified brazzein production strain (Brax8) was cultured in 3 L fermenter for brazzein production. Brax8 seed was inoculated into minimal medium with glycerol and methanol was continually fed into the medium for brazzein induction after glycerol was fully consumed (21 hr). Medium samples were collected at different time points and analyzed by SDS-PAGE. As shown in FIG. 15, no brazzein was detected before methanol feeding (FIG. 15. Lanes 2 and 3); After methanol feeding, brazzein production is increased along with the induction time (FIG. 15. Lanes 4-10).

Brazzein was purified by cation chromatography. Purified brazzein sample was confirmed by LC-MS comparing brazzein standard as described above. In order to confirm 4 intramolecular disulfide bonds, purified brazzein was treated with DTT and analyzed by LC-MS. Compared with untreated sample, the mass of reduction treated sample ([M+H]+: 6375.84m/z) is 8 Dalton higher than untreated sample ([M+H]+: 6367.74m/z), indicating there are four disulfide bonds were reduced to form eight sulfhydryl groups.

Tasing test showed purified brazzein has similar sweetness and taste as reported.

Sequences
Amino acid sequence of brazzein (des-pyrE-bra)
SEQ ID NO: 1
DKCKKVYENYPVSKCQLANQCNYDCKLDKHARSGECFYDEKRNLQCICDYCEY
DNA sequence encoding SEQ ID NO: 1 (brazzein (des-pyrE-bra))
SEQ ID NO: 2
GATAAGTGTAAGAAGGTATATGAGAACTATCCAGTTAGTAAGTGTCAATTGGCCA
ATCAATGCAACTATGACTGCAAGTTGGACAAGCACGCCAGAAGTGGTGAGTGCTT
CTACGATGAGAAGCGGAATCTTCAATGTATCTGCGACTACTGTGAATAC
Amino acid sequence of spacer 1 (KR)
SEQ ID NO: 3
KR
DNA sequence encoding spacer 1
SEQ ID NO: 4
AAGCGA
Amino acid sequence of spacer 2 (KREA)
SEQ ID NO: 5
KREA
DNA sequence encoding spacer 2
SEQ ID NO: 6
AAGCGAGAAGCC
Amino acid sequence of spacer 3
(KREAEAEAEAEA; also referred to herein as KR(EA)5)
SEQ ID NO: 7
KREAEAEAEAEA
DNA sequence encoding spacer 3
SEQ ID NO: 8
AAGCGAGAAGCCGAAGCAGAAGCAGAAGCAGAAGCG
Amino acid sequence of Bra-KRx2
SEQ ID NO: 9
DKCKKVYENYPVSKCQLANQCNYDCKLDKHARSGECFYDEKRNLQCICDYCEYKRD
KCKKVYENYPVSKCQLANQCNYDCKLDKHARSGECFYDEKRNLQCICDYCEY
DNA sequence encoding Bra-KRx2
SEQ ID NO: 10
GATAAGTGTAAGAAGGTATATGAGAACTATCCAGTTAGTAAGTGTCAATTGGCCA
ATCAATGCAACTATGACTGCAAGTTGGACAAGCACGCCAGAAGTGGTGAGTGCTT
CTACGATGAGAAGCGGAATCTTCAATGTATCTGCGACTACTGTGAATACAAGCGA
GATAAGTGTAAGAAGGTATATGAGAACTATCCAGTTAGTAAGTGTCAATTGGCCA
ATCAATGCAACTATGACTGCAAGTTGGACAAGCACGCCAGAAGTGGTGAGTGCTT
CTACGATGAGAAGCGGAATCTTCAATGTATCTGCGACTACTGTGAATAC
Amino acid sequence of Bra-KRx8
SEQ ID NO: 11
DKCKKVYENYPVSKCQLANQCNYDCKLDKHARSGECFYDEKRNLQCICDYCEYKRD
KCKKVYENYPVSKCQLANQCNYDCKLDKHARSGECFYDEKRNLQCICDYCEYKRDK
CKKVYENYPVSKCQLANQCNYDCKLDKHARSGECFYDEKRNLQCICDYCEYKRDKC
KKVYENYPVSKCQLANQCNYDCKLDKHARSGECFYDEKRNLQCICDYCEYKRDKCK
KVYENYPVSKCQLANQCNYDCKLDKHARSGECFYDEKRNLQCICDYCEYKRDKCKK
VYENYPVSKCQLANQCNYDCKLDKHARSGECFYDEKRNLQCICDYCEYKRDKCKKV
YENYPVSKCQLANQCNYDCKLDKHARSGECFYDEKRNLQCICDYCEYKRDKCKKVY
ENYPVSKCQLANQCNYDCKLDKHARSGECFYDEKRNLQCICDYCEY
DNA sequence encoding Bra-KRx8
SEQ ID NO: 12
GATAAGTGTAAGAAGGTATATGAGAACTATCCAGTTAGTAAGTGTCAATTGGCCA
ATCAATGCAACTATGACTGCAAGTTGGACAAGCACGCCAGAAGTGGTGAGTGCTT
CTACGATGAGAAGCGGAATCTTCAATGTATCTGCGACTACTGTGAATACAAGCGA
GATAAGTGTAAGAAGGTATATGAGAACTATCCAGTTAGTAAGTGTCAATTGGCCA
ATCAATGCAACTATGACTGCAAGTTGGACAAGCACGCCAGAAGTGGTGAGTGCTT
CTACGATGAGAAGCGGAATCTTCAATGTATCTGCGACTACTGTGAATACAAGCGA
GATAAGTGTAAGAAGGTATATGAGAACTATCCAGTTAGTAAGTGTCAATTGGCCA
ATCAATGCAACTATGACTGCAAGTTGGACAAGCACGCCAGAAGTGGTGAGTGCTT
CTACGATGAGAAGCGGAATCTTCAATGTATCTGCGACTACTGTGAATACAAGCGA
GATAAGTGTAAGAAGGTATATGAGAACTATCCAGTTAGTAAGTGTCAATTGGCCA
ATCAATGCAACTATGACTGCAAGTTGGACAAGCACGCCAGAAGTGGTGAGTGCTT
CTACGATGAGAAGCGGAATCTTCAATGTATCTGCGACTACTGTGAATACAAGCGA
GATAAGTGTAAGAAGGTATATGAGAACTATCCAGTTAGTAAGTGTCAATTGGCCA
ATCAATGCAACTATGACTGCAAGTTGGACAAGCACGCCAGAAGTGGTGAGTGCTT
CTACGATGAGAAGCGGAATCTTCAATGTATCTGCGACTACTGTGAATACAAGCGA
GATAAGTGTAAGAAGGTATATGAGAACTATCCAGTTAGTAAGTGTCAATTGGCCA
ATCAATGCAACTATGACTGCAAGTTGGACAAGCACGCCAGAAGTGGTGAGTGCTT
CTACGATGAGAAGCGGAATCTTCAATGTATCTGCGACTACTGTGAATACAAGCGA
GATAAGTGTAAGAAGGTATATGAGAACTATCCAGTTAGTAAGTGTCAATTGGCCA
ATCAATGCAACTATGACTGCAAGTTGGACAAGCACGCCAGAAGTGGTGAGTGCTT
CTACGATGAGAAGCGGAATCTTCAATGTATCTGCGACTACTGTGAATACAAGCGA
GATAAGTGTAAGAAGGTATATGAGAACTATCCAGTTAGTAAGTGTCAATTGGCCA
ATCAATGCAACTATGACTGCAAGTTGGACAAGCACGCCAGAAGTGGTGAGTGCTT
CTACGATGAGAAGCGGAATCTTCAATGTATCTGCGACTACTGTGAATAC
Amino acid sequence of Bra-KREAx2
SEQ ID NO: 13
DKCKKVYENYPVSKCQLANQCNYDCKLDKHARSGECFYDEKRNLQCICDYCEYKRE
ADKCKKVYENYPVSKCQLANQCNYDCKLDKHARSGECFYDEKRNLQCICDYCEY
DNA sequence encoding Bra-KREAx2
SEQ ID NO: 14
TAAGTGTAAGAAGGTATATGAGAACTATCCAGTTAGTAAGTGTCAATTGGCCAATC
AATGCAACTATGACTGCAAGTTGGACAAGCACGCCAGAAGTGGTGAGTGCTTCTAC
GATGAGAAGCGGAATCTTCAATGTATCTGCGACTACTGTGAATACAAGCGAGAAGC
CGATAAGTGTAAGAAGGTATATGAGAACTATCCAGTTAGTAAGTGTCAATTGGCCA
ATCAATGCAACTATGACTGCAAGTTGGACAAGCACGCCAGAAGTGGTGAGTGCTTC
TACGATGAGAAGCGGAATCTTCAATGTATCTGCGACTACTGTGAATAC
Amino acid sequence of Bra-KREAx8
SEQ ID NO: 15
DKCKKVYENYPVSKCQLANQCNYDCKLDKHARSGECFYDEKRNLQCICDYCEYKRE
ADKCKKVYENYPVSKCQLANQCNYDCKLDKHARSGECFYDEKRNLQCICDYCEYKR
EADKCKKVYENYPVSKCQLANQCNYDCKLDKHARSGECFYDEKRNLQCICDYCEYK
READKCKKVYENYPVSKCQLANQCNYDCKLDKHARSGECFYDEKRNLQCICDYCEY
KREADKCKKVYENYPVSKCQLANQCNYDCKLDKHARSGECFYDEKRNLQCICDYCE
YKREADKCKKVYENYPVSKCQLANQCNYDCKLDKHARSGECFYDEKRNLQCICDYC
EYKREADKCKKVYENYPVSKCQLANQCNYDCKLDKHARSGECFYDEKRNLQCICDY
CEYKREADKCKKVYENYPVSKCQLANQCNYDCKLDKHARSGECFYDEKRNLQCICD
YCEY
DNA sequence encoding Bra-KREAx8
SEQ ID NO: 16
GATAAGTGTAAGAAGGTATATGAGAACTATCCAGTTAGTAAGTGTCAATTGGCCAA
TCAATGCAACTATGACTGCAAGTTGGACAAGCACGCCAGAAGTGGTGAGTGCTTCT
ACGATGAGAAGCGGAATCTTCAATGTATCTGCGACTACTGTGAATACAAGCGAGAA
GCCGATAAGTGTAAGAAGGTATATGAGAACTATCCAGTTAGTAAGTGTCAATTGGC
CAATCAATGCAACTATGACTGCAAGTTGGACAAGCACGCCAGAAGTGGTGAGTGCT
TCTACGATGAGAAGCGGAATCTTCAATGTATCTGCGACTACTGTGAATACAAGCGA
GAAGCCGATAAGTGTAAGAAGGTATATGAGAACTATCCAGTTAGTAAGTGTCAATT
GGCCAATCAATGCAACTATGACTGCAAGTTGGACAAGCACGCCAGAAGTGGTGAGT
GCTTCTACGATGAGAAGCGGAATCTTCAATGTATCTGCGACTACTGTGAATACAAG
CGAGAAGCCGATAAGTGTAAGAAGGTATATGAGAACTATCCAGTTAGTAAGTGTCA
ATTGGCCAATCAATGCAACTATGACTGCAAGTTGGACAAGCACGCCAGAAGTGGTG
AGTGCTTCTACGATGAGAAGCGGAATCTTCAATGTATCTGCGACTACTGTGAATAC
AAGCGAGAAGCCGATAAGTGTAAGAAGGTATATGAGAACTATCCAGTTAGTAAGTG
TCAATTGGCCAATCAATGCAACTATGACTGCAAGTTGGACAAGCACGCCAGAAGTG
GTGAGTGCTTCTACGATGAGAAGCGGAATCTTCAATGTATCTGCGACTACTGTGAA
TACAAGCGAGAAGCCGATAAGTGTAAGAAGGTATATGAGAACTATCCAGTTAGTAA
GTGTCAATTGGCCAATCAATGCAACTATGACTGCAAGTTGGACAAGCACGCCAGAA
GTGGTGAGTGCTTCTACGATGAGAAGCGGAATCTTCAATGTATCTGCGACTACTGT
GAATACAAGCGAGAAGCCGATAAGTGTAAGAAGGTATATGAGAACTATCCAGTTAG
TAAGTGTCAATTGGCCAATCAATGCAACTATGACTGCAAGTTGGACAAGCACGCCA
GAAGTGGTGAGTGCTTCTACGATGAGAAGCGGAATCTTCAATGTATCTGCGACTAC
TGTGAATACAAGCGAGAAGCCGATAAGTGTAAGAAGGTATATGAGAACTATCCAGT
TAGTAAGTGTCAATTGGCCAATCAATGCAACTATGACTGCAAGTTGGACAAGCACG
CCAGAAGTGGTGAGTGCTTCTACGATGAGAAGCGGAATCTTCAATGTATCTGCGAC
TACTGTGAATAC
Amino acid sequence of Bra-KR(EA)5×2
SEQ ID NO: 17
DKCKKVYENYPVSKCQLANQCNYDCKLDKHARSGECFYDEKRNLQCICDYCEYKRE
AEAEAEAEADKCKKVYENYPVSKCQLANQCNYDCKLDKHARSGECFYDEKRNLQCI
CDYCEY
DNA sequence encoding Bra-KR(EA)5x2
SEQ ID NO: 18
GATAAGTGTAAGAAGGTATATGAGAACTATCCAGTTAGTAAGTGTCAATTGGCCAA
TCAATGCAACTATGACTGCAAGTTGGACAAGCACGCCAGAAGTGGTGAGTGCTTCT
ACGATGAGAAGCGGAATCTTCAATGTATCTGCGACTACTGTGAATACAAGCGAGAA
GCCGAAGCAGAAGCAGAAGCAGAAGCGGATAAGTGTAAGAAGGTATATGAGAACTA
TCCAGTTAGTAAGTGTCAATTGGCCAATCAATGCAACTATGACTGCAAGTTGGACA
AGCACGCCAGAAGTGGTGAGTGCTTCTACGATGAGAAGCGGAATCTTCAATGTATC
TGCGACTACTGTGAATAC
Amino acid sequence of Bra-KR(EA)5x8
SEQ ID NO: 19
DKCKKVYENYPVSKCQLANQCNYDCKLDKHARSGECFYDEKRNLQCICDYCEYKRE
AEAEAEAEADKCKKVYENYPVSKCQLANQCNYDCKLDKHARSGECFYDEKRNLQCI
CDYCEYKREAEAEAEAEADKCKKVYENYPVSKCQLANQCNYDCKLDKHARSGECFY
DEKRNLQCICDYCEYKREAEAEAEAEADKCKKVYENYPVSKCQLANQCNYDCKLDK
HARSGECFYDEKRNLQCICDYCEYKREAEAEAEAEADKCKKVYENYPVSKCQLANQ
CNYDCKLDKHARSGECFYDEKRNLQCICDYCEYKREAEAEAEAEADKCKKVYENYP
VSKCQLANQCNYDCKLDKHARSGECFYDEKRNLQCICDYCEYKREAEAEAEAEADK
CKKVYENYPVSKCQLANQCNYDCKLDKHARSGECFYDEKRNLQCICDYCEYKREAE
AEAEAEADKCKKVYENYPVSKCQLANQCNYDCKLDKHARSGECFYDEKRNLQCICD
YCEY
DNA sequence encoding Bra-KR(EA)5x8
SEQ ID NO: 20
GATAAGTGTAAGAAGGTATATGAGAACTATCCAGTTAGTAAGTGTCAATTGGCCAA
TCAATGCAACTATGACTGCAAGTTGGACAAGCACGCCAGAAGTGGTGAGTGCTTCT
ACGATGAGAAGCGGAATCTTCAATGTATCTGCGACTACTGTGAATACAAGCGAGAA
GCCGAAGCAGAAGCAGAAGCAGAAGCGGATAAGTGTAAGAAGGTATATGAGAACTA
TCCAGTTAGTAAGTGTCAATTGGCCAATCAATGCAACTATGACTGCAAGTTGGACA
AGCACGCCAGAAGTGGTGAGTGCTTCTACGATGAGAAGCGGAATCTTCAATGTATC
TGCGACTACTGTGAATACAAGCGAGAAGCCGAAGCAGAAGCAGAAGCAGAAGCGGA
TAAGTGTAAGAAGGTATATGAGAACTATCCAGTTAGTAAGTGTCAATTGGCCAATC
AATGCAACTATGACTGCAAGTTGGACAAGCACGCCAGAAGTGGTGAGTGCTTCTAC
GATGAGAAGCGGAATCTTCAATGTATCTGCGACTACTGTGAATACAAGCGAGAAGC
CGAAGCAGAAGCAGAAGCAGAAGCGGATAAGTGTAAGAAGGTATATGAGAACTATC
CAGTTAGTAAGTGTCAATTGGCCAATCAATGCAACTATGACTGCAAGTTGGACAAG
CACGCCAGAAGTGGTGAGTGCTTCTACGATGAGAAGCGGAATCTTCAATGTATCTG
CGACTACTGTGAATACAAGCGAGAAGCCGAAGCAGAAGCAGAAGCAGAAGCGGATA
AGTGTAAGAAGGTATATGAGAACTATCCAGTTAGTAAGTGTCAATTGGCCAATCAA
TGCAACTATGACTGCAAGTTGGACAAGCACGCCAGAAGTGGTGAGTGCTTCTACGA
TGAGAAGCGGAATCTTCAATGTATCTGCGACTACTGTGAATACAAGCGAGAAGCCG
AAGCAGAAGCAGAAGCAGAAGCGGATAAGTGTAAGAAGGTATATGAGAACTATCCA
GTTAGTAAGTGTCAATTGGCCAATCAATGCAACTATGACTGCAAGTTGGACAAGCA
CGCCAGAAGTGGTGAGTGCTTCTACGATGAGAAGCGGAATCTTCAATGTATCTGCG
ACTACTGTGAATACAAGCGAGAAGCCGAAGCAGAAGCAGAAGCAGAAGCGGATAAG
TGTAAGAAGGTATATGAGAACTATCCAGTTAGTAAGTGTCAATTGGCCAATCAATG
CAACTATGACTGCAAGTTGGACAAGCACGCCAGAAGTGGTGAGTGCTTCTACGATG
AGAAGCGGAATCTTCAATGTATCTGCGACTACTGTGAATACAAGCGAGAAGCCGAA
GCAGAAGCAGAAGCAGAAGCGGATAAGTGTAAGAAGGTATATGAGAACTATCCAGT
TAGTAAGTGTCAATTGGCCAATCAATGCAACTATGACTGCAAGTTGGACAAGCACG
CCAGAAGTGGTGAGTGCTTCTACGATGAGAAGCGGAATCTTCAATGTATCTGCGAC
TACTGTGAATAC
Amino acid sequence of (PpPDI)
SEQ ID NO: 21
MQFNWDIKTVASILSALTLAQASDQEAIAPEDSHVVKLTEATFESFITSNPHVLAE
FFAPWCGHCKKLGPELVSAAEILKDNEQVKIAQIDCTEEKELCQGYEIKGYPTLKV
FHGEVEVPSDYQGQRQSQSIVSYMLKQSLPPVSEINATKDLDDTIAEAKEPVIVQV
LPEDASNLESNTTFYGVAGTLREKFTFVSTKSTDYAKKYTSDSTPAYLLVRPGEEP
SVYSGEELDETHLVHWIDIESKPLFGDIDGSTFKSYAEANIPLAYYFYENEEQRAA
AADIIKPFAKEQRGKINFVGLDAVKFGKHAKNLNMDEEKLPLFVIHDLVSNKKFGV
PQDQELTNKDVTELIEKFIAGEAEPIVKSEPIPEIQEEKVFKLVGKAHDEVVFDES
KDVLVKYYAPWCGHCKRMAPAYEELATLYANDEDASSKVVIAKLDHTLNDVDNVDI
QGYPTLILYPAGDKSNPQLYDGSRDLESLAEFVKERGTHKVDALALRPVEEEKEAE
EEAESEADAHDEL
DNA sequence encoding (PpPDI)
SEQ ID NO: 22
ATGCAATTCAACTGGGATATTAAAACTGTGGCAAGTATTTTGTCCGCTCTCACACT
AGCACAAGCAAGTGATCAGGAGGCTATTGCTCCAGAGGACTCTCATGTCGTCAAAT
TGACTGAAGCCACTTTTGAGTCTTTCATCACCAGTAATCCTCACGTTTTGGCAGAG
TTTTTTGCCCCTTGGTGTGGTCACTGTAAGAAGTTGGGCCCTGAACTTGTTTCTGC
TGCCGAGATTTTAAAGGACAATGAGCAGGTTAAGATTGCTCAAATTGATTGTACGG
AGGAGAAGGAATTATGTCAAGGCTACGAAATTAAAGGGTATCCTACTTTGAAGGTG
TTCCATGGTGAGGTTGAGGTCCCAAGTGACTATCAAGGTCAAAGACAGAGCCAAAG
CATTGTCAGCTATATGCTAAAGCAGAGTTTACCCCCTGTCAGTGAAATCAATGCAA
CCAAAGATTTAGACGACACAATCGCCGAGGCAAAAGAGCCCGTGATTGTGCAAGTA
CTACCGGAAGATGCATCCAACTTGGAATCTAACACCACATTTTACGGAGTTGCCGG
TACTCTCAGAGAGAAATTCACTTTTGTCTCCACTAAGTCTACTGATTATGCCAAAA
AATACACTAGCGACTCGACTCCTGCCTATTTGCTTGTCAGACCTGGCGAGGAACCT
AGTGTTTACTCTGGTGAGGAGTTAGATGAGACTCATTTGGTGCACTGGATTGATAT
TGAGTCCAAACCTCTATTTGGAGACATTGACGGATCTACCTTCAAATCATACGCTG
AAGCTAACATCCCTTTAGCCTACTATTTCTATGAGAACGAAGAACAACGTGCTGCT
GCTGCCGATATTATTAAACCTTTTGCTAAAGAGCAACGTGGCAAAATTAACTTTGT
TGGCTTAGATGCCGTTAAATTCGGTAAGCATGCCAAGAACTTAAACATGGATGAAG
AGAAACTCCCTCTATTTGTCATTCATGATTTGGTGAGCAACAAGAAGTTTGGAGTT
CCTCAAGACCAAGAATTGACGAACAAAGATGTGACCGAGCTGATTGAGAAATTCAT
CGCAGGAGAGGCAGAACCAATTGTGAAATCAGAGCCAATTCCAGAAATTCAAGAAG
AGAAAGTCTTCAAGCTAGTCGGAAAGGCCCACGATGAAGTTGTCTTCGATGAATCT
AAAGATGTTCTAGTCAAGTACTACGCCCCTTGGTGTGGTCACTGTAAGAGAATGGC
TCCTGCTTATGAGGAATTGGCTACTCTTTACGCCAATGATGAGGATGCCTCTTCAA
AGGTTGTGATTGCAAAACTTGATCACACTTTGAACGATGTTGACAACGTTGATATT
CAAGGTTATCCTACTTTGATCCTTTATCCAGCTGGTGATAAATCCAATCCTCAACT
GTATGATGGATCTCGTGACCTAGAATCATTGGCTGAGTTTGTAAAGGAGAGAGGAA
CCCACAAAGTGGATGCCCTAGCACTCAGACCAGTCGAGGAAGAAAAGGAAGCTGAA
GAAGAAGCTGAAAGTGAGGCAGACGCTCACGACGAGCTTTAA
Amino acid sequence of (AtPDI1)
SEQ ID NO: 23
MASSSTSISLLLFVSFILLLVNSRAENASSGSDLDEELAFLAAEESKEQSHGGGSY
HEEEHDHQHRDFENYDDLEQGGGEFHHGDHGYEEEPLPPVDEKDVAVLTKDNFTEF
VGNNSFAMVEFYAPWCGACQALTPEYAAAATELKGLAALAKIDATEEGDLAQKYEI
QGFPTVFLFVDGEMRKTYEGERTKDGIVTWLKKKASPSIHNITTKEEAERVLSAEP
KLVFGFLNSLVGSESEELAAASRLEDDLSFYQTASPDIAKLFEIETQVKRPALVLL
KKEEEKLARFDGNFTKTAIAEFVSANKVPLVINFTREGASLIFESSVKNQLILFAK
ANESEKHLPTLREVAKSFKGKFVFVYVQMDNEDYGEAVSGFFGVTGAAPKVLVYTG
NEDMRKFILDGELTVNNIKTLAEDFLADKLKPFYKSDPLPENNDGDVKVIVGNNFD
EIVLDESKDVLLEIYAPWCGHCQSFEPIYNKLGKYLKGIDSLVVAKMDGTSNEHPR
AKADGFPTILFFPGGNKSFDPIAVDVDRTVVELYKFLKKHASIPFKLEKPATPEPV
ISTMKSDEKIEGDSSKDEL
DNA sequence encoding (AtPDI1)
SEQ ID NO: 24
ATGGCTTCTTCTTCTACTTCTATTTCTTTGTTGTTGTTCGTTTCTTTCATCTTGTT
GTTGGTTAATTCTAGAGCTGAAAACGCTTCTTCTGGTTCTGATTTGGATGAAGAAT
TGGCTTTTCTTGCTGCTGAAGAATCTAAAGAACAATCTCATGGTGGTGGTTCTTAT
CATGAAGAAGAACATGATCATCAACATAGAGATTTTGAAAACTACGATGATTTGGA
ACAAGGTGGTGGTGAATTTCATCATGGTGACCATGGTTACGAAGAAGAACCATTGC
CACCAGTTGATGAAAAAGATGTTGCTGTTTTGACTAAAGATAACTTCACTGAATTT
GTCGGTAATAACTCTTTCGCTATGGTTGAATTTTACGCTCCATGGTGTGGTGCTTG
TCAAGCTTTGACTCCTGAATATGCTGCTGCTGCTACTGAATTGAAAGGTTTGGCTG
CTTTGGCTAAGATTGATGCTACTGAAGAAGGTGACTTGGCTCAAAAGTATGAAATT
CAAGGTTTTCCTACTGTTTTCTTGTTTGTTGATGGTGAAATGAGAAAGACTTATGA
AGGTGAAAGAACTAAGGATGGTATTGTTACTTGGTTGAAAAAGAAAGCTTCTCCTT
CTATTCATAACATTACTACTAAGGAAGAGGCTGAAAGAGTTTTGTCTGCTGAACCA
AAGTTGGTTTTTGGTTTTCTTAACTCTTTGGTTGGTTCTGAATCTGAAGAATTGGC
CGCTGCTTCTAGATTGGAAGATGATTTGTCTTTTTACCAAACTGCTTCTCCTGATA
TTGCTAAATTGTTCGAAATTGAAACCCAAGTTAAGCGTCCTGCTTTGGTTTTGTTG
AAAAAGGAAGAAGAAAAGTTGGCTAGATTTGATGGTAATTTTACTAAGACTGCTAT
CGCTGAATTTGTTTCTGCTAATAAGGTTCCATTGGTTATTAATTTCACCAGAGAAG
GTGCTTCTTTGATTTTCGAATCTTCTGTTAAGAACCAATTGATTTTGTTCGCTAAA
GCTAATGAATCTGAAAAGCATTTGCCTACTTTGAGAGAAGTTGCTAAGTCTTTCAA
AGGTAAATTCGTTTTCGTTTACGTTCAAATGGATAATGAAGATTACGGTGAAGCTG
TTTCTGGTTTCTTTGGTGTTACTGGTGCTGCTCCAAAGGTTTTGGTTTATACTGGT
AACGAAGATATGAGAAAGTTCATTTTGGATGGTGAATTGACTGTTAACAATATTAA
GACTCTGGCTGAAGATTTTCTTGCTGATAAGTTGAAACCATTCTACAAGTCTGATC
CATTGCCTGAAAACAACGATGGTGACGTTAAGGTTATTGTTGGTAACAACTTCGAT
GAAATTGTTTTGGATGAATCTAAGGATGTTTTGTTGGAAATCTATGCTCCATGGTG
CGGTCATTGTCAATCTTTTGAACCAATCTATAACAAGTTGGGTAAATACTTGAAGG
GTATTGATTCTTTGGTTGTTGCTAAAATGGATGGTACTTCTAACGAACATCCAAGA
GCTAAAGCTGATGGTTTTCCTACCATTTTGTTTTTCCCTGGTGGTAATAAGTCTTT
CGATCCTATTGCTGTTGATGTTGATAGAACTGTTGTTGAATTGTATAAGTTCTTGA
AGAAGCATGCTTCTATTCCTTTCAAGTTGGAAAAGCCAGCTACTCCAGAACCTGTT
ATTTCTACTATGAAGTCTGATGAAAAGATCGAAGGTGACTCTTCTAAGGATGAATT
GTAA
Amino acid sequence of (HAC1)
SEQ ID NO: 25
MPVDSSHKTASPLPPRKRAKTEEEKEQRRVERILRNRRAAHASREKKRRHVEFLEN
HVVDLESALQESAKATNKLKEIQDIIVSRLEALGGTVSDLDLTVPEVDFPKSSDLE
PMSDLSTSSKSEKASTSTRRSLTEDLDEDDVAEYDDEEEDEELPRKMKVLNDKNKS
TSIKQEKLNELPSPLSSDFSDVDEEKSTLTHLKLQQQQQQPVDNYVSTPLSLPEDS
VDFINPGNLKIESDENFLLSSNTLQIKHENDTDYITTAPSGSINDFFNSYDISESN
RLHHPAVMTDSSLHITAGSIGFFSLIGGGESSVAGRRSSVGTYQLTCIAIR
DNA sequence encoding (HAC1)
SEQ ID NO: 26
ATGCCCGTAGATTCTTCTCATAAGACAGCTAGCCCACTTCCACCTCGTAAAAGAGC
AAAGACGGAAGAAGAAAAGGAGCAGCGTCGAGTGGAACGTATCCTACGTAATAGGA
GAGCGGCCCATGCTTCCAGAGAGAAGAAACGAAGACACGTTGAATTTCTGGAAAAC
CACGTCGTCGACCTGGAATCTGCACTTCAAGAATCAGCCAAAGCCACTAACAAGTT
GAAAGAAATACAAGATATCATTGTTTCAAGGTTGGAAGCCTTAGGTGGTACCGTCT
CAGATTTGGATTTAACAGTTCCGGAAGTCGATTTTCCCAAATCTTCTGATTTGGAA
CCCATGTCTGATCTCTCAACTTCTTCGAAATCGGAGAAAGCATCTACATCCACTCG
CAGATCTTTGACTGAGGATCTGGACGAAGATGACGTCGCTGAATATGACGACGAAG
AAGAGGACGAAGAGTTACCCAGGAAAATGAAAGTCTTAAACGACAAAAACAAGAGC
ACATCTATCAAGCAGGAGAAGTTGAATGAACTTCCATCTCCTTTGTCATCCGATTT
TTCAGACGTAGATGAAGAAAAGTCAACTCTCACACATTTAAAGTTGCAACAGCAAC
AACAACAACCAGTAGACAATTATGTTTCTACTCCTTTGAGTCTTCCGGAGGATTCA
GTTGATTTTATTAACCCAGGTAACTTAAAAATAGAGTCCGATGAGAACTTCTTGTT
GAGTTCAAATACTTTACAAATAAAACACGAAAATGACACCGACTACATTACTACAG
CTCCATCAGGTTCCATCAATGATTTTTTTAATTCTTATGACATTAGCGAGTCGAAT
CGGTTGCATCATCCAGCAGTGATGACGGATTCATCTTTACACATTACAGCAGGCTC
CATCGGCTTTTTCTCTTTGATTGGGGGGGGGGAAAGTTCTGTAGCAGGGAGGCGCA
GTTCAGTTGGCACATATCAGTTGACATGCATAGCGATCAGG
Amino acid sequence of (AtERO1)
SEQ ID NO: 27
MGKGAIKEEESEKKRKTWRWPLATLVVVFLAVAVSSRTNSNVGFFFSDRNSCSCSL
QKTGKYKGMIEDCCCDYETVDNLNTEVLNPLLQDLVTTPFFRYYKVKLWCDCPFWP
DDGMCRLRDCSVCECPENEFPEPFKKPFVPGLPSDDLKCQEGKPQGAVDRTIDNRA
FRGWVETKNPWTHDDDTDSGEMSYVNLQLNPERYTGYTGPSARRIWDSIYSENCPK
YSSGETCPEKKVLYKLISGLHSSISMHIAADYLLDESRNQWGQNIELMYDRILRHP
DRVRNMYFTYLFVLRAVTKATAYLEQAEYDTGNHAEDLKTQSLIKQLLYSPKLQTA
CPVPFDEAKLWQGQSGPELKQQIQKQFRNISALMDCVGCEKCRLWGKLQVQGLGTA
LKILFSVGNQDIGDQTLQLQRNEVIALVNLLNRLSESVKMVHDMSPDVERLMEDQI
AKVSAKPARLRRIWDLAVSFW
DNA sequence encoding (AtERO1)
SEQ ID NO: 28
ATGGGTAAAGGTGCTATTAAGGAAGAAGAATCTGAAAAGAAGAGAAAAACTTGGAG
ATGGCCTTTGGCTACTTTGGTTGTTGTTTTCTTGGCTGTTGCTGTTTCTTCTAGAA
CTAACTCTAACGTTGGTTTCTTTTTCTCTGATAGAAATTCTTGTTCCTGTTCTTTG
CAAAAAACTGGTAAATACAAGGGTATGATTGAAGATTGTTGTTGTGATTATGAGAC
TGTTGATAACTTGAATACTGAAGTTTTGAACCCTTTGTTGCAAGATTTGGTTACTA
CTCCATTTTTCAGATACTACAAAGTTAAGTTGTGGTGTGATTGTCCATTCTGGCCA
GATGATGGTATGTGTAGATTGAGAGATTGTTCTGTTTGTGAATGTCCAGAAAACGA
ATTTCCTGAACCATTCAAAAAGCCTTTCGTTCCTGGTTTGCCATCTGATGATTTGA
AATGTCAAGAAGGTAAACCACAAGGTGCTGTTGATAGAACTATTGATAACAGAGCT
TTTAGAGGTTGGGTTGAAACTAAAAACCCTTGGACTCATGATGATGATACTGATTC
TGGTGAAATGTCTTATGTTAATTTGCAATTGAACCCAGAAAGATACACTGGTTACA
CTGGTCCTTCTGCTAGAAGAATTTGGGATTCTATCTATTCTGAAAACTGTCCAAAG
TACTCTTCTGGTGAAACTTGTCCAGAAAAGAAAGTTTTGTATAAGTTGATCTCCGG
TTTGCATTCTTCTATTTCTATGCATATTGCTGCTGATTATTTGTTGGATGAATCTA
GAAATCAGTGGGGTCAAAACATTGAATTGATGTATGATAGAATCCTGAGACATCCA
GATAGAGTTAGAAATATGTATTTCACTTACCTGTTCGTTTTGAGAGCTGTTACTAA
AGCTACTGCTTATTTGGAACAAGCTGAATACGATACTGGTAACCATGCTGAAGATT
TGAAAACTCAATCTTTGATTAAGCAGTTGTTGTATTCTCCTAAATTGCAAACTGCT
TGTCCAGTTCCTTTTGATGAAGCTAAGTIGTGGCAAGGTCAATCTGGTCCAGAATT
GAAACAACAAATTCAAAAACAGTTCAGAAACATCTCTGCTTTGATGGATTGTGTTG
GTTGTGAAAAGTGTAGATTGTGGGGTAAATTGCAAGTTCAAGGTTTGGGTACTGCT
TTGAAAATTTTGTTTTCTGTTGGTAACCAGGATATCGGTGACCAAACTTTGCAATT
GCAAAGAAACGAAGTTATTGCTTTGGTTAATTTGTTGAACAGATTGTCTGAATCTG
TTAAGATGGTTCATGATATGTCTCCAGATGTTGAAAGATTGATGGAAGATCAAATT
GCTAAAGTTTCTGCTAAACCTGCTAGATTGAGAAGAATTTGGGACTTGGCTGTTTC
TTTCTGGTAA
Amino acid sequence of (AtERO2)
SEQ ID NO: 29
MAETDVGSVKGKEKGSGKRWILLIGAIAAVLLAVVVAVFLNTQNSSISEFTGKICN
CRQAEQQKYIGIVEDCCCDYETVNRLNTEVLNPLLQDLVKTPFYRYFKVKLWCDCP
FWPDDGMCRLRDCSVCECPESEFPEVFKKPLSQYNPVCQEGKPQATVDRTLDTRAF
RGWTVTDNPWTSDDETDNDEMTYVNLRLNPERYTGYIGPSARRIWEAIYSENCPKH
TSEGSCQEEKILYKLVSGLHSSISVHIASDYLLDEATNLWGQNLTLLYDRVLRYPD
RVQNLYFTFLFVLRAVTKAEDYLGEAEYETGNVIEDLKTKSLVKQVVSDPKTKAAC
PVPFDEAKLWKGQRGPELKQQLEKQFRNISAIMDCVGCEKCRLWGKLQILGLGTAL
KILFTVNGEDNLRHNLELQRNEVIALMNLLHRLSESVKYVHDMSPAAERIAGGHAS
SGNSFWQRIVTSIAQSKAVSGKRS
DNA sequence encoding (AtERO2)
SEQ ID NO: 30
ATGGCTGAAACTGATGTTGGTTCTGTTAAGGGTAAAGAAAAGGGTTCTGGTAAAAG
ATGGATTTTGTTGATTGGTGCTATTGCTGCTGTTTTGTTGGCTGTTGTTGTTGCTG
TTTTCTTGAACACTCAAAACTCTTCTATTTCTGAGTTTACTGGTAAAATCTGTAAC
TGTAGACAAGCTGAACAACAAAAGTACATTGGTATTGTTGAAGATTGTTGTTGTGA
TTATGAGACTGTTAACAGATTGAACACTGAAGTTTTGAACCCATTGTTGCAAGATT
TGGTTAAGACTCCATTCTACAGATACTTTAAGGTTAAGTTGTGGTGTGATTGTCCT
TTCTGGCCAGATGATGGTATGTGTAGATTGAGAGATTGTTCTGTTTGTGAATGTCC
AGAATCTGAATTTCCTGAAGTTTTCAAGAAACCTTTGTCTCAATATAACCCAGTTT
GTCAAGAAGGTAAACCACAAGCTACTGTTGATAGAACTTTGGATACTAGAGCTTTC
AGAGGTTGGACTGTTACTGATAATCCTTGGACTTCTGATGATGAAACTGATAACGA
TGAAATGACTTATGTTAACTTGAGATTGAACCCAGAAAGATACACTGGTTATATTG
GTCCATCTGCTAGAAGAATTTGGGAAGCTATCTATTCTGAAAATTGTCCAAAACAT
ACCTCTGAAGGTTCTTGTCAAGAAGAAAAGATTTTGTATAAGCTGGTTTCTGGTTT
GCATTCTTCTATTTCCGTTCATATTGCTTCTGATTACTTGTTGGATGAAGCTACTA
ACTTGTGGGGTCAAAACTTGACTTTGTTGTATGATAGAGTTTTGAGATACCCAGAT
AGAGTTCAAAACTTGTACTTTACTTTCTTGTTCGTTTTGAGAGCTGTTACTAAAGC
TGAAGATTACTTGGGTGAAGCTGAATACGAAACTGGTAACGTTATTGAAGATTTGA
AAACTAAATCTCTGGTCAAGCAAGTTGTTTCTGATCCAAAAACTAAGGCTGCTTGT
CCAGTTCCATTTGATGAAGCTAAGTTGTGGAAGGGTCAAAGAGGTCCAGAATTGAA
GCAACAATTGGAAAAGCAATTTCGTAACATTTCTGCTATTATGGATTGTGTTGGTT
GTGAAAAATGTAGATTGTGGGGTAAATTGCAAATTTTGGGTTTGGGTACTGCTTTG
AAAATTTTGTTTACTGTTAACGGTGAGGATAATTTGAGACATAACTTGGAATTGCA
AAGAAACGAAGTTATTGCTTTGATGAATTTGTTGCATAGATTGTCTGAATCTGTTA
AATACGTTCATGATATGTCTCCTGCTGCTGAAAGAATTGCTGGTGGTCATGCTTCT
TCTGGTAATTCTTTTTGGCAAAGAATTGTTACTTCCATTGCTCAATCTAAAGCTGT
TTCTGGTAAAAGATCCTAA
Amino acid sequence of (AtERV1)
SEQ ID NO: 31
MGEKPWQPLLQSFEKLSNCVQTHLSNFIGIKNTPPSSQSTIQNPIISLDSSPPIAT
NSSSLQKLPLKDKSTGPVTKEDLGRATWTFLHTLAAQYPEKPTRQQKKDVKELMTI
LSRMYPCRECADHFKEILRSNPAQAGSQEEFSQWLCHVHNTVNRSLGKLVFPCERV
DARWGKLECEQKSCDLHGTSMDF
DNA sequence encoding (AtERV1)
SEQ ID NO: 32
ATGGGTGAAAAACCATGGCAACCATTGTTGCAATCTTTCGAAAAGTTGTCTAATTG
TGTTCAAACTCATTTGTCTAACTTCATTGGTATTAAGAACACTCCACCATCTTCTC
AATCTACTATTCAAAACCCTATTATCTCTTTGGATTCTTCTCCACCAATTGCTACT
AATTCTTCTTCTTTGCAAAAGTTGCCTTTGAAGGATAAGTCTACTGGTCCAGTTAC
TAAGGAAGATTTGGGTAGAGCTACTTGGACTTTTCTTCATACTTTGGCTGCTCAAT
ACCCTGAAAAACCTACTAGACAACAAAAGAAAGATGTTAAGGAATTGATGACTATC
TTGTCTAGAATGTATCCATGTAGAGAATGTGCTGATCATTTCAAAGAAATTTTGAG
ATCCAACCCTGCTCAAGCTGGTTCTCAAGAAGAATTTTCTCAATGGTTGTGTCATG
TTCATAACACTGTTAATAGATCCTTGGGTAAATTGGTTTTCCCTTGTGAAAGAGTT
GATGCTAGATGGGGTAAATTGGAATGTGAACAAAAATCTTGTGACTTGCATGGTAC
TTCTATGGATTTTTAA
Amino acid sequence of (PpERV2)
SEQ ID NO: 33
MIKFNKRVATLTATLLSFIVLYTLFNSGARFANQLDQPVPLKTPELIIPNQSTKND
APLPFMPKMANETLKAELGNASWKLFHTILARYPESPSENQKSTLNDYIYLFAQVY
PCGDCARHFNLLLQKYPPQLSSRQVAAVWGCHIHNQVNKRLEKPQYDCSNILEDYD
CGCGSDEKEVDDTLNNETMEHLQSIKITEKENEQFGR
DNA sequence encoding (PpERV2)
SEQ ID NO: 34
ATGATAACATTCAACAAACGAATAGCAACATTAGCGGCAACGTTATTTTCATTCAT
TGTGCTTTATACTCTCTTTAACAGTGGTGCTCAATTTTCCAACCAACTAGATCAGC
CTGTTCCCCTCAAAACTCCAGAACTCATCATACCGAATCAGAGTACTGAGAATGAT
CCCCCTCTTCCATTCATGCCAAAAATGGCTAACGAAACTTTGAAAGCAGAACTTGG
AAATGCTTCCTGGAAACTCTTTCACACTATTCTTGCTAGATATCCTGAATCCCCAT
CGGAGAATCAAAAATCAACCTTAAATGACTACATTTATTTGTTTGCACAGGTTTAT
CCATGTGGAGACTGTGCAAGACATTTCAATTTATTGCTGCAGAAATACCCTCCACA
ATTGTCCTCAAGACAGGTGGCTGCAGTGTGGGGATGTCATATTCACAATCAGGTCA
ATAAGAGATTGGAGAAACCACAATACGACTGCTCCAATATTCTAGAGGATTACGAT
TGTGGATGTGGCTCTGATGAAAAGGAAGTAGATGACACTCTGAATAACGAAACAAT
AGAACACTTGCAAAGTATCAAAATTACTGAAAAAGAGAGTGAACAATTTGGTCGA
Amino acid sequence of (PpERO1)
SEQ ID NO: 35
MRIVRSLAVTITCYCITALANPQIPFDGNYTEITVPDTEVNIGQIVDINHEIKPKL
VELVNTDFFKYYKLNLWKPCPFWNGDEGFCKYKDCSVDFITDWSQVPDIWQPDQLG
KLGDNTVHKDKGQDENELSSNDYCALDKDDDEDLVYVNLIDNPERFTGYGGQQSES
IWTAVYDENCFQPNEGSQLGQVEDLCLEKQIFYRLVSGLHSSISTHLTNEYLNLKN
GEYEPNLKQFMIKVGYFTERIQNLHLNYVLVLKSLIKLQEYNVIENLPLDDSLKAG
LSGLISQGAQNINQTDDYLFNEKVLFQNDQNDDLKNEFRDKFRNVTRLMDCVHCER
CKLWGKLQTTGYGTALKILFDLKNPNDSINLKRVELVALVNTFHRLSKSVESIENF
EKLYKIQPPTQDHPSPSSESLDVFDNEDEQNFFDSFSVDQTVTSSKEPPEEIKSKP
VGKAEYKKTNSCPSSGSKSIKEAFHEELYAFIDAIGFILNSYRTLPKLLYTLFLVK
SSELWDIFIGTQRHRDSTYRVDL
DNA sequence encoding (PpERO1)
SEQ ID NO: 36
ATGAGGATAGTAAGGAGCGTAGCTATCGCAATAGCCTGTCATTGTATAACAGCGTT
AGCAAACCCTCAAATCCCTTTTGACGGCAACTACACCGAGATCATCGTGCCAGATA
CCGAAGTTAACATCGGACAGATTGTAGATATTAACCACGAAATAAAACCCAAACTG
GTGGAACTGGTCAACACAGACTTCTTCAAATATTACAAATTAAACCTATGGAAACC
ATGTCCGTTTTGGAATGGTGATGAGGGATTCTGCAAGTATAAGGATTGCTCTGTTG
ACTTTATCACTGATTGGTCCCAGGTGCCTGATATCTGGCAACCAGACCAATTGGGT
AAGCTTGGAGATAACACGGTACATAAGGATAAGGGCCAAGATGAAAATGAGCTGTC
CTCAAATGATTATTGCGCTTTGGATAAAGACGACGATGAAGATTTAGTATATGTCA
ATTTGATTGATAACCCTGAAAGATTCACCGGTTATGGTGGTCAGCAATCTGAATCT
ATTTGGACTGCGGTCTATGATGAGAACTGTTTCCAGCCGAATGAAGGATCACAATT
GGGTCAAGTTGAAGACCTCTGTTTGGAGAAACAAATCTTTTACCGATTGGTTTCTG
GTTTGCATTCTAGTATCTCCACCCACCTCACAAACGAATATCTGAATTTGAAAAAT
GGAGCATACGAACCAAATTTGAAACAGTTCATGATCAAAGTTGGGTATTTTACTGA
AAGAATCCAAAACTTACATCTCAATTATGTCCTTGTATTGAAGTCACTAATAAAGC
TACAAGAATACAATGTTATCGACAATCTACCTCTCGATGACTCTTTGAAAGCTGGT
CTTAGCGGTTTAATATCTCAAGGAGCACAGGGTATTAACCAGAGTTCTGATGATTA
TCTATTTAACGAGAAGGTTCTTTTCCAAAATGACCAAAATGATGATTTGAAAAATG
AATTTCGTGACAAATTCCGCAACGTGACTAGATTAATGGATTGTGTCCATTGCGAG
AGATGCAAATTATGGGGAAAATTGCAAACTACAGGGTACGGGACTGCATTGAAGAT
TCTATTTGATTTGAAGAATCCTAATGACTCCATCAATTTAAAGAGAGTTGAGTTAG
TTGCTCTAGTCAACACATTCCATAGATTGTCCAAATCTGTTGAAAGCATTGAAAAC
TTTGAAAAACTATATAAGATTCAACCGCCAACGCAGGATCGTGCATCAGCGTCGTC
CGAATCCTTAGGCCTTTTCGATAACGAAGATGAACAAAATCTCCTCAACTCGTTTT
CGGTTGATCAGGCAGTCATTTCATCGAAAGAGGCACCAGAAGAAATCAAAAGCAAA
CCTGTTGGAAAAGCCGCATATAAACAAAACAGTTGTCCATCATTGGGTTCAAAATC
TATCAAAGAAGCATTCCATGAAGAACTTCACGCATTTATTGATGCAATTGGATTTA
TATTGAACTCTTACAGGACTTTGCCCAAGCTGTTGTACACACTTTTCCTCGTTAAA
TCATCTGAATTATGGGACATTTTCATTGGCACTCAAAGGCACCGAGATACCACATA
TAGAGTAGACTTGTAAGCGGCCGCCAGCTT
Amino acid sequence of (PpKAR2)
SEQ ID NO: 37
MLSLKPSWLTLAALMYAMLLVVVPFAKPVRADDVESYGTVIGIDLGTTYSCVGVMK
SGRVEILANDQGNRITPSYVSFTEDERLVGDAAKNLAASNPKNTIFDIKRLIGMKY
DAPEVQRDLKRLPYTVKSKNGQPVVSVEYKGEEKSFTPEEISAMVLGKMKLIAEDY
LGKKVTHAVVTVPAYFNDAQRQATKDAGLIAGLTVLRIVNEPTAAALAYGLDKTGE
ERQIIVYDLGGGTFDVSLLSIEGGAFEVLATAGDTHLGGEDFDYRVVRHFVKIFKK
KHNIDISNNDKALGKLKREVEKAKRTLSSQMTTRIEIDSFVDGIDFSEQLSRAKFE
EINIELFKKTLKPVEQVLKDAGVKKSEIDDIVLVGGSTRIPKVQQLLEDYFDGKKA
SKGINPDEAVAYGAAVQAGVLSGEEGVDDIVLLDVNPLTLGIETTGGVMTTLINRN
TAIPTKKSQIFSTAADNQPTVLIQVYEGERALAKDNNLLGKFELTGIPPAPRGTPQ
VEVTFVLDANGILKVSATDKGTGKSESITINNDRGRLSKEEVDRMVEEAEKYAAED
AALREKIEARNALENYAHSLRNQVTDDSETGLGSKLDEDDKETLTDAIKDTLEFLE
DNFDTATKEELDEQREKLSKIAYPITSKLYGAPEGGTPPGGQGFDDDDGDFDYDYD
YDHDEL
DNA sequence encoding (PpKAR2)
SEQ ID NO: 38
ATGCTGTCGTTAAAACCATCTTGGCTGACTTTGGCGGCATTAATGTATGCCATGCT
ATTGGTCGTAGTGCCATTTGCTAAACCTGTTAGAGCTGACGATGTCGAATCTTATG
GAACAGTGATTGGTATCGATTTGGGTACCACGTACTCTTGTGTCGGTGTGATGAAG
TCGGGTCGTGTAGAAATTCTTGCTAATGACCAAGGTAACAGAATCACTCCTTCCTA
CGTTAGTTTCACTGAAGACGAGAGACTGGTTGGTGATGCTGCTAAGAACTTAGCTG
CTTCTAACCCAAAAAACACCATCTTTGATATTAAGAGATTGATCGGTATGAAGTAT
GATGCCCCAGAGGTCCAAAGAGACTTGAAGCGTCTTCCTTACACTGTCAAGAGCAA
GAACGGCCAACCTGTCGTTTCTGTCGAGTACAAGGGTGAGGAGAAGTCTTTCACTC
CTGAGGAGATTTCCGCCATGGTCTTGGGTAAGATGAAGTTGATCGCTGAGGACTAC
TTAGGAAAGAAAGTCACTCATGCTGTCGTTACCGTTCCAGCCTACTTCAACGACGC
TCAACGTCAAGCCACTAAGGATGCCGGTCTGATCGCCGGTTTGACTGTTCTGAGAA
TTGTGAACGAGCCTACCGCCGCTGCCCTTGCTTACGGTTTGGACAAGACTGGTGAG
GAAAGACAGATCATCGTCTACGACTTGGGTGGAGGAACCTTCGATGTTTCTCTGCT
TTCTATTGAGGGTGGTGCTTTCGAGGTTCTTGCTACCGCCGGTGACACCCACTTGG
GTGGTGAGGACTTTGACTACAGAGTTGTTCGCCACTTCGTTAAGATTTTCAAGAAG
AAGCATAACATTGACATCAGCAACAATGATAAGGCTTTAGGTAAGCTGAAGAGAGA
GGTCGAAAAGGCCAAGCGTACTTTGTCTTCCCAGATGACTACCAGAATTGAGATTG
ACTCTTTCGTTGACGGTATCGACTTCTCTGAGCAACTGTCTAGAGCTAAGTTTGAG
GAGATCAACATTGAATTATTCAAGAAGACACTGAAACCAGTTGAACAAGTCCTCAA
AGACGCTGGTGTCAAGAAATCTGAAATTGATGACATTGTCTTGGTTGGTGGTTCTA
CCAGAATCCCAAAGGTTCAACAATTATTGGAGGATTACTTTGACGGAAAGAAGGCT
TCTAAGGGAATTAACCCAGATGAAGCTGTCGCATACGGTGCTGCTGTTCAGGCTGG
TGTTTTGTCTGGTGAGGAAGGTGTCGATGACATCGTCTTGCTTGATGTGAACCCCC
TAACTCTGGGTATCGAGACTACTGGTGGCGTTATGACTACCTTAATCAACAGAAAC
ACTGCTATCCCAACTAAGAAATCTCAAATTTTCTCCACTGCTGCTGACAACCAGCC
AACTGTGTTGATTCAAGTTTATGAGGGTGAGAGAGCCTTGGCTAAGGACAACAACT
TGCTTGGTAAATTCGAGCTGACTGGTATTCCACCAGCTCCAAGAGGTACTCCTCAA
GTTGAGGTTACTTTTGTTTTAGACGCTAACGGAATTTTGAAGGTTTCTGCCACCGA
TAAGGGAACTGGAAAATCCGAGTCCATCACCATCAACAATGATCGTGGTAGATTGT
CCAAGGAGGAGGTTGACCGTATGGTTGAAGAGGCCGAGAAGTACGCCGCTGAGGAT
GCTGCACTAAGAGAAAAGATTGAGGCTAGAAACGCTCTGGAGAACTACGCTCATTC
CCTTAGGAACCAAGTTACTGATGACTCTGAAACCGGGCTTGGTTCTAAATTGGACG
AGGACGACAAAGAGACATTGACAGATGCCATCAAAGATACCCTAGAGTTCTTGGAA
GACAACTTCGACACCGCAACCAAGGAAGAATTAGACGAACAAAGAGAAAAGCTTTC
CAAGATTGCTTACCCAATCACTTCTAAGCTATACGGTGCTCCAGAGGGTGGTACTC
CACCTGGTGGTCAAGGTTTTGACGATGATGATGGAGACTTTGACTACGACTATGAC
TATGATCATGATGAGTTGTAA
Amino acid sequence of (PpSEC1)
SEQ ID NO: 39
MDLVKVGQSYVDKIVTDTGIKVLLLDDITSSIISLVSTQSELLNHQVYLIDKLENE
NRDTIKQLDCVCFLSVSEKTINLLVEELGAPKYKSYKLYFNNVVPNSFLERLAERD
DLEMVDKVMELFLDYDILNKNLFSFKQLNIFNSIDAWNQQQFLLTLASLKSLCFSL
QTNPIIRYESNSRMCSKLASDLSYEFGQSSKIMEKFPVNDIPPVLLILDRKNDPIT
PLLNPWTYQSMVHELLGIFNNTVDLTGTPSDLPPDLIKLVLNPSQDPFYAQSLYLN
FGDLSDSIKTYVNEYKEKTVKHNSNELTDLNDMKHFLESFPEFKKLSNNISKHMGL
ITELDRKINENHLWQVSELEQSIAVNDNHNADLQELEKLLTSQEFKIANNLKVKLV
CLYAIRYELHPNNQLPKMLSILLQQGVPEFEINTVNRMLKYSGSTKRLNDDSESSI
FNQATNNLLQGFKQSHENDNIYMQHIPRLERVISKLVKNKLPTAHYPTLINDFLKK
QRPVSDLNGARLQDIIIFFVGGVTYEEARIINNFNLVNKSTRIVIGGTTVHNTNSF
MTQVLELE
DNA sequence encoding (PpSEC1)
SEQ ID NO: 40
ATGGACTTGGTTAAGGTTGGACAATCCTACGTGGATAAAATTGTCACAGACACAGG
CATTAAGGTTCTTTTATTGGATGATATCACTTCTTCCATAATTTCCCTAGTGAGCA
CCCAATCAGAATTGTTGAACCATCAGGTGTATTTGATCGACAAGTTGGAGAACGAG
AATAGAGATACGATAAAGCAATTGGATTGTGTGTGTTTCCTATCAGTATCAGAAAA
AACTATAAACTTGCTTGTTGAGGAATTAGGTGCTCCCAAATACAAATCCTACAAGC
TCTACTTCAATAATGTAGTTCCCAACTCATTCTTAGAGAGGTTGGCGGAGAGGGAC
GATTTGGAAATGGTCGATAAGGTCATGGAATTGTTCCTAGATTACGACATTTTGAA
CAAGAACTTGTTTTCCTTCAAACAACTGAATATTTTCAATTCAATTGATGCTTGGA
ATCAGCAACAGTTTCTCTTGACTTTAGCAAGCTTGAAATCACTCTGCTTCTCCTTG
CAAACGAATCCTATAATCAGGTATGAATCTAATAGTCGAATGTGTTCTAAGCTAGC
TTCCGATTTGTCATACGAATTTGGGCAAAGTTCTAAAATTATGGAAAAGTTCCCGG
TGAATGATATCCCTCCTGTCCTGTTAATTCTTGACCGAAAAAACGACCCAATCACT
CCATTATTAAATCCTTGGACTTATCAATCTATGGTACACGAGCTTTTAGGAATTTT
CAATAATACGGTGGATTTAACGGGAACTCCTTCTGATCTGCCCCCAGACCTAATCA
AACTGGTATTGAATCCCTCTCAAGATCCATTTTATGCTCAGTCTCTATATTTGAAT
TTCGGAGACTTGTCCGATAGTATAAAAACATACGTAAACGAGTACAAAGAAAAAAC
CGTCAAACACAATTCTAATGAATTGACAGATTTGAATGATATGAAACACTTTCTGG
AATCTTTTCCAGAGTTCAAAAAACTTTCAAACAACATTTCCAAACACATGGGCTTG
ATTACAGAATTAGATAGAAAAATCAACGAAAATCACTTATGGCAAGTGAGTGAATT
GGAACAATCCATAGCTGTTAATGACAATCATAATGCTGACCTTCAAGAACTAGAAA
AGCTGTTGACATCTCAAGAGTTCAAGATTGCCAACAACTTAAAAGTTAAATTAGTA
TGTTTGTATGCCATACGATATGAACTTCATCCCAACAACCAGCTTCCAAAAATGTT
GTCAATACTTTTACAGCAGGGGGTGCCAGAGTTTGAAATAAATACAGTCAACAGGA
TGTTGAAATACTCGGGAAGTACCAAACGATTGAATGATGACTCTGAATCTTCGATA
TTTAACCAGGCAACAAATAATCTACTGCAGGGGTTCAAACAAAGTCATGAAAACGA
CAATATTTATATGCAGCATATTCCAAGGTTGGAAAGAGTTATCAGCAAGTTAGTGA
AAAATAAGCTACCCACAGCGCATTATCCGACTTTAATCAATGATTTTTTGAAGAAG
CAACGCCCTGTTTCTGATCTAAATGGAGCCAGGCTGCAAGATATTATTATTTTCTT
TGTTGGTGGAGTCACTTATGAAGAGGCCCGAATAATTAACAATTTCAATCTGGTGA
ACAAGTCTACGAGGATAGTTATAGGGGGAACTACAGTACACAACACGAATAGTTTT
ATGACTCAAGTTCTAGAATTGGAGTAA
Amino acid sequence of (PpSLY1)
SEQ ID NO: 41
MSFTTSLPSLRDRQIATLEKMLHLNEPIVDNGSDIQAELTWKVLILDSRSTAIVSS
VLRVNDLLSSGITMHSNIRSKRAALPDVPVIYFVEPNAENINFIDDLERDQYAHFY
INFTSSLNRDLLEEFAKKVATIGKSYKIKQVYDQYLDYIVTEPNLFSLDLVNIYSQ
LNNPNSLEDEINKVADKISNGIFAAILTMNGIPTIRCCRGGPAELIASKLDQKLRD
HVINTKSSASFTNSKLVLILLDRNIDLASMFAHSWIYQCMVSDVFELKRNTIKIPS
QKPNESTKEYDIDPKDFFWAANNSLPFPDAVENVENELSRYKADAAELTRKTGVSS
LQDIDPNAITDTTDIQLAVKSLPELAFRKSILDMHMKVLASLLQELESKSLDSYFE
IEQNYKDPKNQKQFISILNNGNEHTLNDKLRTYIMLYLLTDLPGSFVEECEEYFKK
NSAELGSLSYIKRAKEVIKLSNYELSMSIDASHSTTSGLVNEAQKSALFQGLSSKL
YGLTDGGSRLTEGVGSLITGLKNLLPDKKQLPITNIVESIMEPSLATQESIKLTDD
YLYFDPISTRGVHSKPPKRQQYNNSIVFVVGGGNYLEYQNLQEWVTKTNTSNVNGT
KSVIYGSTSIVTANEFLKECSLLGAEAK
DNA sequence encoding (PpSLY1)
SEQ ID NO: 42
ATGCTTCATTTGAATGAGCCCATTGTGGATAATGGTTCAGATATACAAGCGGAGTT
AACATGGAAGGTACTGATTCTGGATAGTAGGAGTACTGCAATTGTTTCTTCTGTTC
TGCGAGTTAATGACCTGCTTTCTTCTGGCATCACTATGCATAGCAATATCAGATCC
AAGAGAGCGGCTTTGCCAGATGTTCCTGTCATTTACTTTGTTGAACCTAATGCGGA
AAATATCAACTTTATCATTGATGACTTGGAAAGAGATCAGTACGCTCATTTTTATA
TCAACTTCACTTCCAGTCTAAATAGGGACCTTTTGGAGGAGTTTGCTAAGAAAGTG
GCTACGATTGGTAAGTCCTACAAGATTAAACAGGTTTATGATCAGTACCTCGATTA
CATTGTCACTGAACCCAACCTGTTCTCTTTGGACTTGGTTAACATTTACTCGCAGC
TAAATAACCCTAACTCACTGGAAGATGAAATCAATAAAGTTGCTGACAAGATTTCC
AATGGTATATTCGCAGCAATCCTAACTATGAATGGTATCCCTACTATTAGATGTTG
CAGAGGAGGTCCAGCAGAACTAATAGCGTCCAAACTAGATCAGAAGCTACGTGATC
ATGTTATCAATACAAAGTCATCTGCCTCTTTCACTAACAGTAAATTAGTGCTTATC
CTGCTGGATAGAAACATTGATTTGGCTTCCATGTTTGCTCATTCATGGATTTATCA
ATGTATGGTGAGTGATGTTTTTGAGTTGAAAAGAAATACAATCAAAATTCCCTCTC
AAAAGCCCAATGAATCTACGAAAGAATATGATATCGACCCAAAGGATTTTTTTTGG
GCAGCCAACAACAGTTTGCCCTTCCCTGATGCTGTAGAAAATGTGGAGAACGAACT
TTCTAGATACAAAGCGGATGCTGCAGAGCTAACTAGAAAGACTGGGGTTTCTTCTC
TTCAAGATATTGATCCCAATGCAATTACTGACACCACAGATATACAGCTTGCTGTG
AAGTCTTTACCTGAATTGGCTTTTAGAAAAAGCATCCTTGATATGCACATGAAAGT
ACTTGCGTCTTTGCTGCAAGAACTGGAATCAAAGTCATTGGATTCATACTTTGAAA
TTGAACAAAACTACAAAGATCCCAAAAACCAGAAGCAGTTTATCAGTATCCTCAAC
AACGGGAATGAGCATACCTTGAACGACAAACTGAGAACCTACATCATGTTGTATCT
GTTAACAGACCTCCCAGGGTCGTTCGTTGAAGAATGTGAAGAGTATTTCAAAAAGA
ACTCCGCTGAGCTTGGTTCGTTGAGTTATATCAAGCGGGCAAAAGAGGTGATCAAG
TTGTCTAATTATGAGTTGTCCATGTCAATTGATGCTAGCCACTCGACCACTAGTGG
ATTGGTGAATGAAGCTCAAAAGTCTGCTTTGTTCCAAGGATTGTCGTCCAAGCTAT
ATGGATTAACAGATGGTGGTAGTAGGCTTACAGAGGGGGTGGGGTCATTAATTACT
GGGTTGAAAAACTTGCTACCCGACAAGAAACAACTGCCTATTACCAATATTGTTGA
ATCGATAATGGAACCAAGTCTGGCCACTCAAGAGTCGATAAAACTAACGGACGATT
ACCTATATTTTGACCCTATTAGCACAAGAGGAGTTCACTCCAAACCACCCAAAAGA
CAGCAATACAACAATTCTATTGTGTTTGTTGTAGGAGGGGGCAACTATTTGGAGTA
CCAAAATTTGCAAGAATGGGTTACGAAGACCAATACTAGCAACGTCAATGGCACTA
AGTCTGTAATCTACGGTAGTACCAGTATCGTGACCGCGAACGAGTTCTTGAAGGAG
TGCTCCTTGCTCGGTGCCGAAGCAAAATAA
Amino acid sequence of (PpGPX1)
SEQ ID NO: 43
MSSFYDLAPLDKKGEPFPFEQLKGKVVLIVNVASKCGFTPQYTELEKLYKDHKDEG
LTIVGFPCNQFGHQEPGNDEEIGQFCQLNFGVTFPILKKIDVNGSEADPVYEFLKS
KKSGLLGFKGIKWNFEKFLIDKQGNVIERYSSLTKPSSIESKIEELLKK
DNA sequence encoding (PpGPX1)
SEQ ID NO: 44
ATGTCTTCATTTTATGATCTGGCCCCATTAGATAAGAAAGGCGAACCTTTTCCTTT
CGAACAATTAAAAGGCAAAGTGGTGTTGATTGTGAATGTTGCTTCTAAGTGTGGGT
TTACTCCACAATATACCGAGTTGGAAAAGCTCTACAAAGACCACAAGGACGAGGGA
TTGACTATTGTCGGATTTCCCTGTAACCAGTTTGGTCATCAGGAACCAGGAAATGA
TGAAGAAATTGGACAGTTTTGCCAGTTGAATTTTGGTGTAACTTTCCCAATTCTAA
AAAAGATTGATGTCAACGGTTCGGAAGCTGATCCTGTTTACGAATTTCTCAAGTCA
AAAAAGTCTGGTCTGCTCGGATTCAAAGGTATTAAGTGGAACTTTGAAAAATTCTT
GATCGATAAGCAAGGAAACGTTATTGAGAGATATTCGTCCTTGACTAAGCCCTCAT
CGATCGAGTCCAAGATTGAAGAACTATTAAAGAAATAA
Amino acid sequence of alpha mating factor signal peptide
SEQ ID NO: 45
MRFPSIFTAVLFAASSALAAPVNTTTEDETAQIPAEAVIGYSDLEGDFDVAVLPFS
NSTNNGLLFINTTIASIAAKEEGVSLEKR
DNA sequence encoding alpha mating factor signal peptide
SEQ ID NO: 46
ATGAGATTTCCTTCAATTTTTACTGCTGTTTTATTCGCAGCATCCTCCGCATTAGC
TGCTCCAGTCAACACTACAACAGAAGATGAAACGGCACAAATTCCGGCTGAAGCTG
TCATCGGTTACTCAGATTTAGAAGGGGATTTCGATGTTGCTGTTTTGCCATTTTCC
AACAGCACAAATAACGGGTTATTGTTTATAAATACTACTATTGCCAGCATTGCTGC
TAAAGAAGAAGGGGTATCTCTCGAGAAAAGA
Amino acid sequence of spacer 4
(KREAEA, also referred to herein as KR(EA)2)
SEQ ID NO: 47
KREAEA
DNA sequence encoding spacer 4
SEQ ID NO: 48
AAGCGAGAAGCCGAAGCA
Amino acid sequence of spacer 5
(KREAEAEA, also referred to herein as KR(EA)3)
SEQ ID NO: 49
KREAEAEA
DNA sequence encoding spacer 5
SEQ ID NO: 50
AAGCGAGAAGCAGAAGCAGAAGCG
Amino acid sequence of spacer 6
(KREAEAEAEA, also referred to herein as KR(EA)4)
SEQ ID NO: 51
KREAEAEAEA
SEQ ID NO: 52
DNA sequence encoding spacer 6
AAGCGAGAAGCAGAAGCAGAAGCAGAAGCG
Amino acid sequence of fusion protein Bra-KREAEAx2
SEQ ID NO: 53
DKCKKVYENYPVSKCQLANQCNYDCKLDKHARSGECFYDEKRNLQCICDYCEYKRE
AEADKCKKVYENYPVSKCQLANQCNYDCKLDKHARSGECFYDEKRNLQCICDYCEY
DNA sequence encoding fusion protein Bra-KREAEAx2
SEQ ID NO: 54
GATAAGTGTAAGAAGGTATATGAGAACTATCCAGTTAGTAAGTGTCAATTGGCCAA
TCAATGCAACTATGACTGCAAGTTGGACAAGCACGCCAGAAGTGGTGAGTGCTTCT
ACGATGAGAAGCGGAATCTTCAATGTATCTGCGACTACTGTGAATACAAGCGAGAA
GCCGAAGCAGATAAGTGTAAGAAGGTATATGAGAACTATCCAGTTAGTAAGTGTCA
ATTGGCCAATCAATGCAACTATGACTGCAAGTTGGACAAGCACGCCAGAAGTGGTG
AGTGCTTCTACGATGAGAAGCGGAATCTTCAATGTATCTGCGACTACTGTGAATAC
Amino acid sequence of fusion protein Bra-KR(EA)3x2
SEQ ID NO: 55
DKCKKVYENYPVSKCQLANQCNYDCKLDKHARSGECFYDEKRNLQCICDYCEYKRE
AEAEADKCKKVYENYPVSKCQLANQCNYDCKLDKHARSGECFYDEKRNLQCICDYC
EY
DNA sequence encoding fusion protein Bra-KR(EA)3x2
SEQ ID NO: 56
GATAAGTGTAAGAAGGTATATGAGAACTATCCAGTTAGTAAGTGTCAATTGGCCAA
TCAATGCAACTATGACTGCAAGTTGGACAAGCACGCCAGAAGTGGTGAGTGCTTCT
ACGATGAGAAGCGGAATCTTCAATGTATCTGCGACTACTGTGAATACAAGCGAGAA
GCAGAAGCAGAAGCGGATAAGTGTAAGAAGGTATATGAGAACTATCCAGTTAGTAA
GTGTCAATTGGCCAATCAATGCAACTATGACTGCAAGTTGGACAAGCACGCCAGAA
GTGGTGAGTGCTTCTACGATGAGAAGCGGAATCTTCAATGTATCTGCGACTACTGT
GAATAC
Amino acid sequence of fusion protein Bra-KR(EA)4x2
SEQ ID NO: 57
DKCKKVYENYPVSKCQLANQCNYDCKLDKHARSGECFYDEKRNLQCICDYCEYKRE
AEAEAEADKCKKVYENYPVSKCQLANQCNYDCKLDKHARSGECFYDEKRNLQCICD
YCEY
DNA sequence of fusion protein Bra-KR(EA)4x2
SEQ ID NO: 58
GATAAGTGTAAGAAGGTATATGAGAACTATCCAGTTAGTAAGTGTCAATTGGCCAA
TCAATGCAACTATGACTGCAAGTTGGACAAGCACGCCAGAAGTGGTGAGTGCTTCT
ACGATGAGAAGCGGAATCTTCAATGTATCTGCGACTACTGTGAATACAAGCGAGAA
GCAGAAGCAGAAGCAGAAGCGGATAAGTGTAAGAAGGTATATGAGAACTATCCAGT
TAGTAAGTGTCAATTGGCCAATCAATGCAACTATGACTGCAAGTTGGACAAGCACG
CCAGAAGTGGTGAGTGCTTCTACGATGAGAAGCGGAATCTTCAATGTATCTGCGAC
TACTGTGAATAC

### REFERENCES

1. Ding Ming. Göran Hellekant. Brazzein, a new high-potency thermostable sweet protein from Pentadiplandra brazzcana B., FEBS Letters, Volume 355, Issue 1,1994, Pages 106-108.
2. Göran Hellekant, Vicktoria Danilova, Brazzein a Small, Sweet Protein: Discovery and Physiological Overview, Chemical Senses, Volume 30, Issue suppl_1, January 2005, Pages i88-i89, https://doi.org/10.1093/chemse/bjh127
3. Izawa, H., Ota, M., Kohmura, M. and Ariyoshi, Y. (1996) Synthesis and characterization of the sweet protein Brazzein. Biopolymers, 39, 95-101.

1. A method of producing brazzein, the method comprising culturing a recombinant yeast cell comprising a polynucleotide encoding a polypeptide comprising tandem repeats of brazzein.

2. The method of claim 1, wherein each repeat of brazzein comprises the amino acid sequence of SEQ ID NO: 1.

3. The method of claim 1, wherein each brazzein in the tandem repeats is separated by a spacer.

4. The method of claim 3, wherein the spacer comprises a protease cleavage site for a yeast protease and/or the amino acid sequence of any one of SEQ ID NOs: 3, 5, 7, 47, 49, and 51.

5. (canceled)

6. The method of claim 1, wherein the polypeptide comprises 2-8 repeats of brazzein; and/or an amino acid sequence at least 80% identical to or comprises the amino acid sequence of any one of SEQ ID NOs: 9, 11, 13, 15, 17, 19, 53, 55, and 57.

7-8. (canceled)

9. The method of claim 1, wherein the polypeptide further comprises an N-terminal signal peptide, optionally wherein the signal peptide is a yeast alpha mating factor signal peptide, optionally wherein the yeast alpha mating factor signal peptide comprises the amino acid sequence of SEQ ID NO: 45.

10. (canceled)

11. The method of claim 1, wherein the polynucleotide is operably linked to a promoter and/or a transcription terminator, optionally wherein the promoter is an AOX1 promoter and/or the transcription terminator is an AOX1 terminator.

12. (canceled)

13. The method of claim 1, wherein the polynucleotide is provided on a vector, optionally wherein the vector is a plasmid.

14. The method of claim 1, wherein the polynucleotide is integrated into the genome of the recombinant yeast cell, optionally wherein the polynucleotide is integrated into a HIS4 locus of the genome of the recombinant yeast cell.

15. The method of claim 1, wherein the recombinant yeast cell further comprises one or more polynucleotides encoding one or more chaperones selected from: PD1, PDI1, HAC1, ERO1, ERO2, ERV1, ERV2, ERO1, KAR2, SEC1, SLY1, and GPX1, optionally wherein the recombinant yeast cell further comprises a polynucleotide encoding PD1, a polynucleotide encoding HAC1, one or more polynucleotides encoding PD1, ERO1, and ERV2, or one or more polynucleotides encoding PD1, ECR1, and ERO1.

16. The method of claim 15, wherein the one or more polynucleotides encoding the one or more chaperones are provided on one or more vectors.

17. The method of claim 15, wherein the one or more polynucleotides encoding the one or more chaperones are integrated into the genome of the recombinant yeast cell, optionally wherein the one or more polynucleotides are integrated into a HIS4 locus of the genome of the recombinant yeast cell.

18. The method of claim 1, wherein the recombinant yeast cell further comprises one or more polynucleotides encoding one or more proteases selected from KEX1, KEX2, and Ste13.

19. The method of claim 18, wherein the one or more polynucleotides encoding the one or more proteases are provided on one or more vectors.

20. The method of claim 18, wherein the one or more polynucleotides encoding the one or more proteases are integrated into the genome of the recombinant yeast cell, optionally wherein the one or more polynucleotides are integrated into a HIS4 locus of the genome of the recombinant yeast cell.

21-23. (canceled)

24. A composition comprising the brazzein produced using the method of claim 1.

25. A consumable product comprising the brazzein produced using the method of claim 1.

26-31. (canceled)

32. A polynucleotide comprising the nucleotide sequence of any one of SEQ ID NOS: 2, 10, 12, 14, 16, 18, 20, 24, 28, 30, 32, 54, 56, and 58.

33. A polypeptide comprising an amino acid sequence that is at least 80% identical to or comprises any one of SEQ ID NOs: 9, 11, 13, 15, 17, 19, 53, 55, and 57.

34. (canceled)

35. A recombinant yeast cell comprising a polynucleotide encoding a polypeptide comprising tandem repeats of brazzein.

36-38. (canceled)
